(19) United States
(12) Patent Application Publication
(10) Pub. No.: US 2026/0021085 A1
(43) Pub. Date: Jan. 22, 2026

(54) CYCLOALKYL CARBOXYLIC ACID DERIVATIVES AS INHIBITORS OF GLYCOGEN SYNTHASE 1 (GYS1) AND METHODS OF USE THEREOF

(71) Applicant: Maze Therapeutics, Inc., South San Francisco, CA (US)

(72) Inventors: David John MORGANS, JR., Los Altos, CA (US); Kevin MELLEM, Redwood City, CA (US); Hannah L. POWERS, San Diego, CA (US); Patrick Sang Tae LEE, Walnut Creek, CA (US); Walter WON, San Diego, CA (US); Christopher Joseph SINZ, Walnut Creek, CA (US)

(21) Appl. No.: 19/111,736

(22) PCT Filed: Sep. 13, 2023
(86) PCT No.: PCT/US2023/074107

§ 371 Date: Mar. 13, 2025

Related U.S. Application Data
(60) Provisional application No. 63/406,682, filed on Sep. 14, 2022.

(51) Int. Cl. A61K 31/4439; A61K 31/192; A61K 31/381; A61K 31/397; A61K 31/404; A61K 31/4184; A61K 31/423; A61K 31/44; A61K 31/4704; C07C 235/82; C07D 205/04; C07D 209/34; C07D 213/61; C07D 215/227; C07D 235/26; C07D 263/58; C07D 333/20; C07D 401/10; C07D 413/06; C07D 413/10

(52) U.S. Cl. CPC A61K 31/4439; A61K 31/192; A61K 31/381; A61K 31/397; A61K 31/404; A61K 31/4184; A61K 31/423; A61K 31/44; A61K 31/4704; C07C 235/82; C07D 205/04; C07D 209/34; C07D 213/61; C07D 215/227; C07D 235/26; C07D 263/58; C07D 333/20; C07D 401/10; C07D 413/06; C07D 413/10

(57) ABSTRACT

Provided herein are compounds of formula (I): or a stereoisomer or tautomer thereof, or a pharmaceutically acceptable salt of any of the foregoing, wherein m, n, Y¹, Y², X¹, X², X³, Q¹, and Ra are as defined elsewhere herein. Also provided herein are methods of preparing compounds of formula (I). Also provided herein are methods of inhibiting GYSI and methods of treating a GYS1-mediated disease, disorder, or condition in an individual in need thereof.

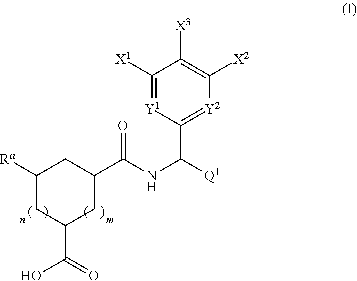

### CROSS REFERENCE TO RELATED APPLICATIONS

This application claims priority to U.S. Provisional Application No. 63/406,682, filed on Sep. 14, 2022, the entire content of which is incorporated herein by reference for all purposes.

### BACKGROUND OF THE INVENTION

Pathological accumulation of glycogen is a hallmark of several devastating and chronic human diseases. For some of these disorders, the cellular etiology driving this aberrant accumulation has clear genetic underpinnings and for others the mechanistic driving force is more complex. Nonetheless, the consequence of elevated levels of glycogen is altered cellular homeostasis and impaired tissue function over time. The rate limiting enzyme in the glycogen synthesis pathway is the protein Glycogen Synthase (GYS). In humans there are two isoforms GYS1 & GYS2. The former is ubiquitously expressed but highly abundant in muscle cells, while the latter is expressed exclusively in liver. Glycogen synthesis ultimately begins with transport of glucose into cells via the GLUT transporter family of proteins. Conversion of glucose into glycogen follows along a well characterized biochemical conversion pathway to the step where GYS covalently links glucose molecules into long branches via α1,4-glycosidic linkages. The final spherical structure of glycogen results from the action of Glycogen Branching Enzyme (GBE) which introduces α1,6-linkage branch points along the strands. The result of this biochemical chain of events is the generation of an energy dense and highly soluble molecule that can be stored in the cytosol of cells for rapid catabolism into glucose energy when needed. An imbalance in the equilibrium of either glycogen synthesis or glycogenolysis can result in aberrant accumulation of cellular stores of glycogen. It has long been hypothesized that substrate reduction therapy targeted to inhibit glycogen synthase could be an effective treatment for diseases of glycogen storage. Indeed, substrate reduction therapy drugs have been very successful in modulating patient disease course in other storage disorders including Gaucher and Fabry diseases (Platt F M, Butters T D. Substrate Reduction Therapy. Lysosomal Storage Disorders, Springer US chapter 11, pgs 153-168, 2007; Shemesh E, et al. Enzyme replacement and substrate reduction therapy for Gaucher disease. Cochrane Database of Systematic Reviews, Issue 3, 2015). It is the aim of this invention to inhibit glycogen synthase enzyme activity resulting in reduction of tissue glycogen stores with therapeutic benefit to patients suffering the consequences of aberrant cellular glycogen accumulation.

Pompe Disease is a rare genetic disorder caused by the pathological buildup of cellular glycogen due to loss of function (LOF) mutations in the lysosomal enzyme α-glucosidase (GAA). GAA catabolizes lysosomal glycogen and in its absence, glycogen builds up in lysosomes. This triggers a disease cascade beginning with lysosome and autophagosome dysfunction, leading ultimately to cell death and muscle atrophy over time (Raben N, et al. Autophagy and mitochondria in Pompe Disease: nothing is so new as what has long been forgotten. American Journal o Medical Genetics, vol. 160, 2012. van der Ploeg A T and Reuser A J J, Pompe's Disease. Lancet vol. 372, 2008). In humans, the clinical manifestation of the disease results in a spectrum of severity and occurs at a prevalence of one in 40,000 live births (Meena N K, Raben N. Pompe disease: new developments in an old lysosomal storage disorder. Biomolecules, vol. 10, 2020). Infantile onset patients are born with cellular pathology and rapidly develop severe impairments including myopathy, heart defects, organomegaly, and hypotonia which collectively left untreated will take the child's life within a year. The later onset children may develop heart enlargement but are characterized consistently by the progressive loss of motor function, degeneration of skeletal muscle, and ultimate failure of the respiratory system leading to early death. Late onset adult Pompe patients exhibit normal heart function but develop progressive muscle weakness and respiratory decline then failure. The current standard of care for Pompe patients is enzyme replacement therapy (ERT) with recombinant human GAA. ERT treatment has been successful in slowing the rate of disease progression but in the majority of patients there remains incredible unmet need (Schoser B, et al. The humanistic burden of Pompe disease: are there still unmet needs? A systematic review: BMC Neurology, vol. 17, 2017). For over a decade, substrate reduction therapy targeting GYS1 has been hypothesized to be beneficial for the treatment of Pompe disease. In fact, three separate preclinical modalities have demonstrated that GYS1 genetic LOF in Pompe model mice effectively reduces tissue glycogen and improves mouse disease outcomes (Douillard-Guilloux G, et al. Modulation of glycogen synthesis by RNA interference: towards a new therapeutic approach for glycogenosis type II. Human Molecular Genetics, vol. 17, no. 24, 2008; Douillard-Guilloux G, et al. Restoration of muscle functionality by genetic suppression of glycogen synthesis in a murine model of Pompe disease. Human Molecular Genetics, vol. 19, no. 4, 2010; Clayton N P, et al. Antisense oligonucleotide-mediated suppression of muscle glycogen synthase I synthesis as an approach for substrate reduction therapy of Pompe Disease. Molecular Therapy-Nucleic Acids, vol. 3, 2014). A small molecule GYS1 inhibitor could be used to address the current unmet needs for Pompe patients either as a single therapy or in combination with standard of care ERT.

Pompe disease is only one of more than a dozen diseases caused by an inborn error of metabolism that result in aberrant build-up of glycogen in various tissues of the body. For some glycogen storage diseases (GSDs), specific dietary regimes effectively manage the disease but for others there are no clinically approved therapeutic interventions to modify disease course. Therefore, inhibition of glycogen synthesis and the concomitant reduction in tissue glycogen levels may be a viable treatment option for these patients. Cori disease, GSD III, is caused by mutations in the glycogen debranching enzyme (GDE) which results in pathological glycogen accumulation in the heart, skeletal muscle, and liver (Kishnani P, et al. Glycogen storage disease type III diagnosis and management guidelines. Genetics in Medicine, vol. 12, no. 7, 2010). While dietary management can be effective in ameliorating aspects of the disease there is currently no treatment to prevent the progressive myopathy in GSD III. Adult polyglucosan body disease (APBD) is an adult-onset disorder caused by loss of activity in the glycogen branching enzyme (GBE1). Deficiency in GBE results in accumulation of long strands of unbranched glycogen which precipitate in the cytosol generating polyglucosan bodies, and ultimately triggering neurological deficits in both the central and peripheral nervous systems. Genetic deletion of GYS1 in the APBD mouse model rescued deleterious accumulation of glycogen, improved life span, and neuromuscular function (Chown E E, et al. GYS1 or PPP1R3C deficiency rescues murine adult polyglucosan body disease. Annals of Clinical and Translational Neurology, vol. 7, no. 11, 2020). Lafora Disease (LD) is a very debilitating juvenile onset epilepsy disorder also characterized by accumulation of polyglucason bodies. Genetic cross of LD mouse models with GYS1 knock out (KO) mice resulted in rescue of disease phenotypes (Pedersen B, et al. Inhibiting glycogen synthesis prevents Lafora disease in a mouse model. Annals of Neurology, vol. 74, no. 2, 2013; Varea O, et al. Suppression of glycogen synthesis as a treatment for Lafora disease: establishing the window of opportunity. Neurobiology of Disease, 2020).

The reliance on high levels of glycogen by clear cell cancers has recently emerged as a novel therapeutic target. Ewing sarcoma (ES), clear cell renal cell carcinoma (ccRCC), glycogen rich clear cell carcinoma breast cancer (GRCC), acute myeloid leukemia (AML), and nonsmall-cell lung carcinoma (NSCLC) are all examples of cancers histopathologically defined by PAS+ abnormally high levels of cellular glycogen. Elevated transcriptional levels of GYS1 have been significantly correlated with poor disease outcomes in NSCLC (Giatromanolaki A, et al. Expression of enzymes related to glucose metabolism in non-small cell lung cancer and prognosis. Experimental Lung Research, vol. 43, no. 4-5, 2017) and AML (Falantes J F, et al. Overexpression of GYS1, MIF, and MYC is associated with adverse outcome and poor response to azacitidine in myelodysplastic syndromes and acute myeloid leukemia. Clinical Lymphoma, Myeloma & Leukemia, vol. 15, no. 4, 2015). Lentiviral knockdown of GYS1 in cultured myeloid leukemia cells potently inhibited in vitro cancer cell growth and in vivo tumorigenesis (Bhanot H, et al. Pathological glycogenesis through glycogen synthase I and suppression of excessive AMP kinase activity in myeloid leukemia cells. Leukemia, vol. 29, no. 7, 2015). Genetic knock-down of GYS1 in ccRCC cell models both suppresses tumor growth in vivo and increases the synthetic lethality of sunitinub (Chen S, et al. GYS1 induces glycogen accumulation and promotes tumor progression via the NF-κB pathway in clear cell renal carcinoma. Theranostics, vol. 10, no. 20, 2020).

Reduction of GYS1 enzyme activity and reduced cellular stores of glycogen in preclinical models of Pompe disease, APBD, LD, AML, ccRCC, and NSCLC all provide compelling evidence of the potential therapeutic benefit of inhibiting glycogen synthesis. It is the aim of this invention to inhibit glycogen synthase enzyme activity resulting in reduction of tissue glycogen stores with therapeutic benefit to patients suffering the consequences of accumulated cellular glycogen.

### BRIEF SUMMARY OF THE INVENTION

In one aspect, provided herein is a compound of formula (I):

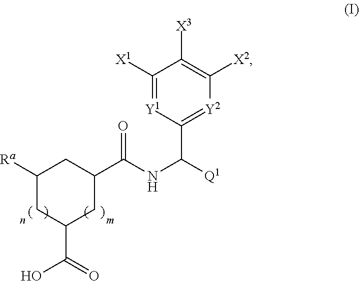

or a stereoisomer or tautomer thereof, or a pharmaceutically acceptable salt of any of the foregoing, wherein:
m is 0, or 1, and n is 0, 1, or 2, wherein m+n is an integer from 1 to 2;
Y¹ and Y² are each CH, or one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl; and
R^{a} is H, halo, —OH, or —NH—C(O)—C₁₋₆alkoxy.

In one aspect, provided herein is a compound of formula (I-A):

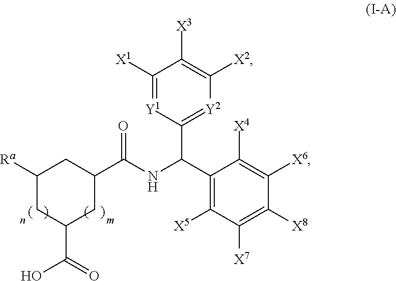

or a stereoisomer or tautomer thereof, or a pharmaceutically acceptable salt of any of the foregoing, wherein m, n, X¹, X², X³, X⁴, X⁵, X⁶, X⁷, X⁸, Y¹, Y², and R^{a} are as defined elsewhere herein.

In one aspect, provided herein is a compound of formula (I-B):

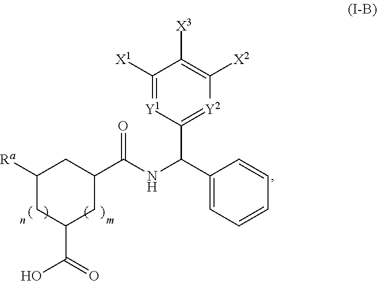

or a stereoisomer or tautomer thereof, or a pharmaceutically acceptable salt of any of the foregoing, wherein m, n, X¹, X², X³, Y¹, Y², and R^{a} are as defined elsewhere herein.

In one aspect, provided herein is a compound of formula (I-C):

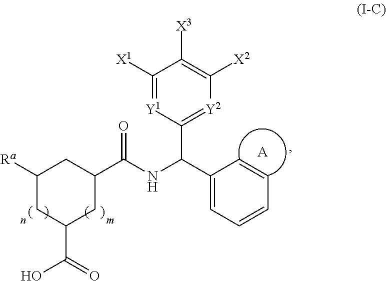

or a stereoisomer or tautomer thereof, or a pharmaceutically acceptable salt of any of the foregoing, wherein m, n, X¹, X², X³, Y¹, Y², R^{a} and ring A are as defined elsewhere herein.

In one aspect, provided herein is a compound of formula (I-D):

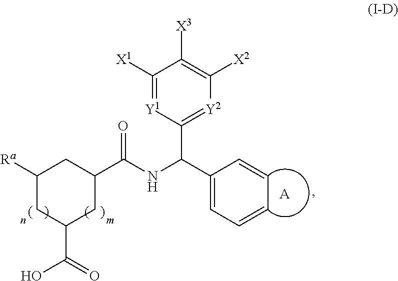

or a stereoisomer or tautomer thereof, or a pharmaceutically acceptable salt of any of the foregoing, wherein m, n, X¹, X², X³, Y¹, Y², R^{a} and ring A are as defined elsewhere herein.

In one aspect, provided is a compound of formula (I-E):

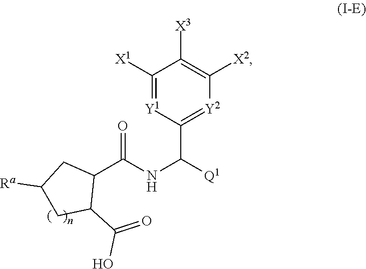

or a stereoisomer or tautomer thereof, or a pharmaceutically acceptable salt of any of the foregoing, wherein n, X¹, X², X³, Y¹, Y², R^{a} and Q¹ are as defined elsewhere herein.

In one aspect, provided is a compound of formula (I-F):

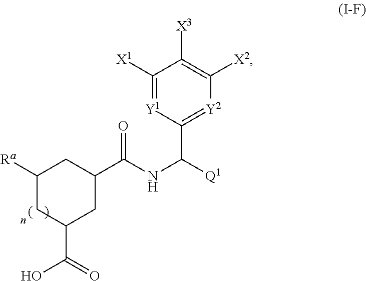

or a stereoisomer or tautomer thereof, or a pharmaceutically acceptable salt of any of the foregoing, wherein n, X¹, X², X³, Y¹, Y², R^{a} and Q¹ are as defined elsewhere herein.

In one aspect, provided herein is a compound of formula (I-G):

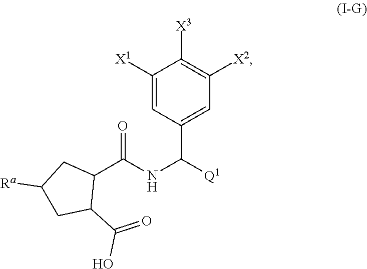

or a stereoisomer or tautomer thereof, or a pharmaceutically acceptable salt of any of the foregoing, wherein X¹, X², X³, Y¹, Y², R^{a} and Q¹ are as defined elsewhere herein.

In one aspect, provided herein is a compound of formula (I-H):

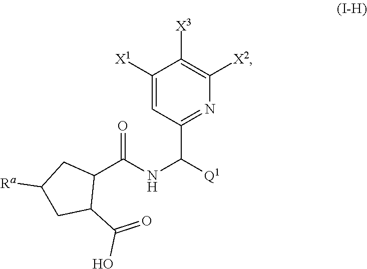

or a stereoisomer or tautomer thereof, or a pharmaceutically acceptable salt of any of the foregoing, wherein X¹, X², X³, Y¹, Y², R^{a} and Q¹ are as defined elsewhere herein.

In one aspect, provided herein is a pharmaceutical composition, comprising (i) a compound of formula (I), or any variation or embodiment thereof, or a stereoisomer or tautomer thereof, or a pharmaceutically acceptable salt of any of the foregoing, and (ii) one or more pharmaceutically acceptable excipients.

In one aspect, provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of inhibiting GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of reducing tissue glycogen stores in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of modulating GYS1 in a cell of an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a GYS1-mediated disease, disorder, or condition in an individual in need thereof, comprising administering to the individual an effective amount of (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a method of treating a GYS1-mediated disease, disorder, or condition in an individual in need thereof, comprising administering to the individual (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients.

In one aspect, provided herein is a kit, comprising (i) a composition comprising an effective amount of a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an GYS1-mediated disease, disorder, or condition in an individual in need thereof.

In one aspect, provided herein is a kit, comprising (i) a compound of formula (I), or any variation or embodiment thereof, or a stereoisomer or tautomer thereof, or a pharmaceutically acceptable salt of any of the foregoing, or a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients, and (ii) instructions for use in treating an GYS1-mediated disease, disorder, or condition in an individual in need thereof.

In some aspect, provided herein are methods of preparing a compound of formula (I), or any embodiment or variation thereof, such as a compound of formula (I), (I-A), (I-A1), (I-A2), (I-B), (I-B1), (I-B2), (I-B3), (I-B4), (I-B5), (I-C), (I-D), (I-E), (I-E1), (I-E2), (I-F), (I-F1), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing.

### BRIEF DESCRIPTION OF THE DRAWINGS

FIG. 1 depicts the pathway in which PPP1R3A Loss of Function (LoF) leads to reduction in muscle glycogen.

FIGS. 2A and 2B depict the association between PPP1R3A protein truncating variant (PTV) and left ventricular ejection (LVEF) (%) and left ventricle wall thickness (mm) in UK Biobank.

FIGS. 2C and 2D depict the association between PPP1R3A protein truncating variant (PTV) and exercise output (watts) and max heart rate (HR) exercise (bpm) in UK Biobank.

FIGS. 2E and 2F depict the association between PPP1R3A protein truncating variant (PTV) and PQ interval (ms) and QRS duration (ms) in UK Biobank.

FIGS. 2G and 2H depict the association between PPP1R3A protein truncating variant (PTV) and QT interval (ms) and serum glucose (mmol/L) in UK Biobank.

### DETAILED DESCRIPTION OF THE INVENTION

“Individual” refers to mammals and includes humans and non-human mammals. Examples of individuals include, but are not limited to, mice, rats, hamsters, guinea pigs, pigs, rabbits, cats, dogs, goats, sheep, cows, and humans. In some embodiments, individual refers to a human.

As used herein, “about” a parameter or value includes and describes that parameter or value per se. For example, “about X” includes and describes X per se.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition: slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated univalent hydrocarbon chain. As used herein, alkyl has 1-20 carbons (i.e., C₁₋₂₀alkyl), 1-16 carbons (i.e., C₁₋₁₆alkyl), 1-12 carbons (i.e., C₁₋₁₂alkyl), 1-10 carbons (i.e., C₁₋₁₀alkyl), 1-8 carbons (i.e., C₁₋₈alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkenyl”, as used herein, refers to a branched or unbranched univalent hydrocarbon chain comprising at least one carbon-carbon double bond. As used herein, alkenyl has 2-20 carbons (i.e., C₂₋₂₀alkenyl), 2-16 carbons (i.e., C₂₋₁₆alkenyl), 2-12 carbons (i.e., C₂₋₁₂alkenyl), 2-10 carbons (i.e., C₂₋₁₀alkenyl), 2-8 carbons (i.e., C₂₋₈alkenyl), 2-6 carbons (i.e., C₂₋₆alkenyl), 2-4 carbons (i.e., C₂₋₄alkenyl), or 2-3 carbons (i.e., C₂₋₃alkenyl). Examples of alkenyl include, but are not limited to, ethenyl, prop-1-enyl, prop-2-enyl 1,2-butadienyl, and 1,3-butadienyl. When an alkenyl residue having a specific number of carbons is named by chemical name or molecular formula, all positional isomers having that number of carbon atoms may be encompassed—for example, “propenyl” includes prop-1-enyl and prop-2-enyl. Certain commonly used alternative names may be used and will be understood by those of ordinary skill in the art. For instance, a divalent group, such as a divalent “alkenyl” group, may be referred to as an “alkenylene”.

The term “alkynyl”, as used herein, refers to a branched or unbranched univalent hydrocarbon chain comprising at least one carbon-carbon triple bond. As used herein, alkynyl has 2-20 carbons (i.e., C₂₋₂₀alkynyl), 2-16 carbons (i.e., C₂₋₁₆alkynyl), 2-12 carbons (i.e., C₂₋₁₂alkynyl), 2-10 carbons (i.e., C₂₋₁₀alkynyl), 2-8 carbons (i.e., C₂₋₈alkynyl), 2-6 carbons (i.e., C₂₋₆alkynyl), 2-4 carbons (i.e., C₂₋₄alkynyl), or 2-3 carbons (i.e., C₂₋₃alkynyl). Examples of alkynyl include, but are not limited to, ethynyl, prop-1-ynyl, prop-2-ynyl, but-1-ynyl, but-2-ynyl, and but-3-ynyl. When an alkynyl residue having a specific number of carbons is named by chemical name or molecular formula, all positional isomers having that number of carbon atoms may be encompassed—for example, “propynyl” includes prop-1-ynyl and prop-2-ynyl. Certain commonly used alternative names may be used and will be understood by those of ordinary skill in the art. For instance, a divalent group, such as a divalent “alkynyl” group, may be referred to as an “alkynylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond. Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₈cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbonyl, decalinyl, 7,7-dimethyl-bicyclo[2.2.1]heptanyl, and the like. Still further, cycloalkyl also includes spiro cycloalkyl ring moieties, for example, spiro[2.5]octanyl, spiro[4.5]decanyl, or spiro[5.5]undecanyl.

The term “halo”, as used herein, refers to atoms occupying groups VIIA of The Periodic Table and includes fluorine (fluoro), chlorine (chloro), bromine (bromo), and iodine (iodo).

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl(benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The term “oxo”, as used herein, refers to a ═O moiety.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties. Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri (iso-propyl)amine, tri (n-propyl)amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In some embodiments, a compound of formula (A) is provided wherein one or more hydrogen is replaced by deuterium or tritium.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vitro or in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

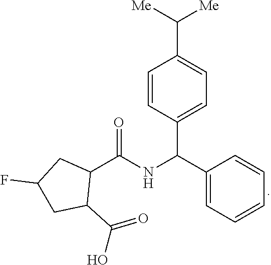 [SMILES: CC(C)c1ccc(C(NC(=O)C2CC(F)CC2C(=O)O)c2ccccc2)cc1]

Where enantiomeric and/or diastereomeric forms exist of a given structure, flat bonds and the presence of a “*” symbol indicate that the composition is made up of at least 90%, by weight, of a single isomer with unknown stereochemistry, e.g.,

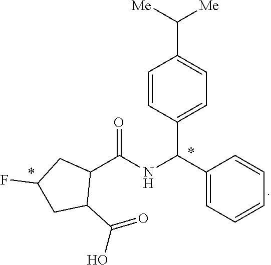 [SMILES: CC(C)c1ccc(C(NC(=O)C2CC(F)CC2C(=O)O)c2ccccc2)cc1]

Where enantiomeric and/or diastereomeric forms exist of a given structure, wedged or hashed bonds indicate the composition is made up of at least 90%, by weight, of a single enantiomer or diastereomer with known stereochemistry, e.g.,

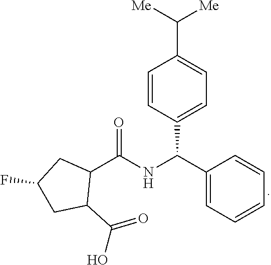 [SMILES: CC(C)c1ccc([C@@H](NC(=O)C2C[C@@H](F)CC2C(=O)O)c2ccccc2)cc1]

Where relevant, combinations of the above notation may be used. Exemplified species may contain stereogenic centers with known stereochemistry and stereogenic centers with unknown stereochemistry, e.g.,

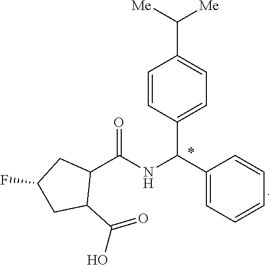 [SMILES: CC(C)c1ccc(C(NC(=O)C2C[C@@H](F)CC2C(=O)O)c2ccccc2)cc1]

Where relevant, combinations of the above notation may be used. Exemplified species may contain stereogenic centers with known stereochemistry and stereogenic centers with unknown stereochemistry, e.g.,

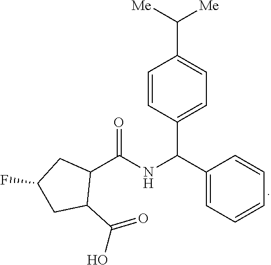 [SMILES: CC(C)c1ccc(C(NC(=O)C2C[C@@H](F)CC2C(=O)O)c2ccccc2)cc1]

### Compounds

In one aspect, provided is a compound of formula (I):

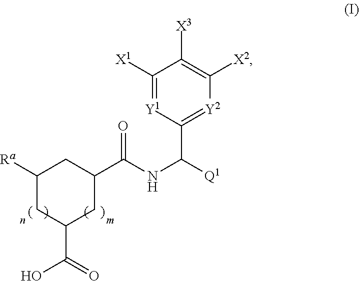

or a stereoisomer or tautomer thereof, or a pharmaceutically acceptable salt of any of the foregoing, wherein:
m is 0, or 1, and n is 0, 1, or 2, wherein m+n is an integer from 1 to 2;
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1, provided that, when X³ is H, then Q¹ is C₆₋₁₀cycloalkyl, wherein the C₆₋₁₀cycloalkyl of Q¹ is optionally substituted with one or more C₁₋₆alkyl; and
R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2. In some embodiments, m is 0, and n is 1, or 2. In some embodiments, m is 0, and n is 1. In some embodiments, m is 0, and n is 2. In some embodiments, m is 1, and n is 0, or 1. In some embodiments, m is 1, and n is 0. In some embodiments, m is 1, and n is 1. In some embodiments, m+n is 1. In some embodiments, m+n is 2.

In some embodiments of a compound of formula (I), or a stereoisomer or tautomer thereof, or a pharmaceutically acceptable salt of any of the foregoing, X¹ and X² are each independently H or halo. In some embodiments, X¹ and X² are each independently H or F. In some embodiments, X¹ and X² are each independently H. In some embodiments, X¹ and X² are each independently halo. In some embodiments, X¹ and X² are each independently F. In some embodiments, one of X¹ and X² is H and the other of X¹ and X² is halo. In some embodiments, one of X¹ and X² is H and the other of X¹ and X² is F.

In some embodiments of a compound of formula (I), or a stereoisomer or tautomer thereof, or a pharmaceutically acceptable salt of any of the foregoing, X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, X³ is H, C₁₋₃alkyl, or C₃₋₆cycloalkyl, wherein the C₃₋₆cycloalkyl of X³ is optionally substituted with one or more C₁₋₃alkyl. In some embodiments, X³ is H, isopropyl, or cyclopropyl, wherein the cyclopropyl of X³ is optionally substituted with one or more methyl. In some embodiments, X³ is H. In some embodiments, X³ is isopropyl. In some embodiments, X³ is cyclopropyl, wherein the cyclopropyl of X³ is optionally substituted with one or more methyl. In some embodiments, X³ is cyclopropyl.

In some embodiments of a compound of formula (I), or a stereoisomer or tautomer thereof, or a pharmaceutically acceptable salt of any of the foregoing, X¹, X², and X³ are each H.

In some embodiments of a compound of formula (I), or a stereoisomer or tautomer thereof, or a pharmaceutically acceptable salt of any of the foregoing, Y¹ and Y² are each CH, or one of Y¹ and Y² is N and the other of Y¹ and Y² is CH.

In some embodiments of a compound of formula (I), or a stereoisomer or tautomer thereof, or a pharmaceutically acceptable salt of any of the foregoing, Y¹ and Y² are each CH, and X¹, X², and X³ are each H.

In some embodiments of a compound of formula (I), or a stereoisomer or tautomer thereof, or a pharmaceutically acceptable salt of any of the foregoing, Y¹ and Y² are each CH.

In some embodiments of a compound of formula (I), or a stereoisomer or tautomer thereof, or a pharmaceutically acceptable salt of any of the foregoing,

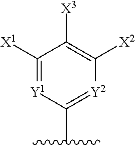

of formula (I) is selected from the group consisting of

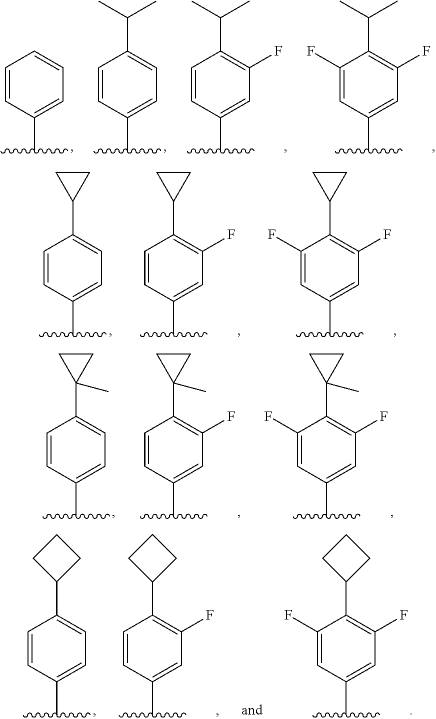

In some embodiments,

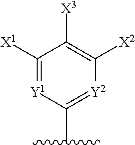

of formula (I) is selected from the group consisting of

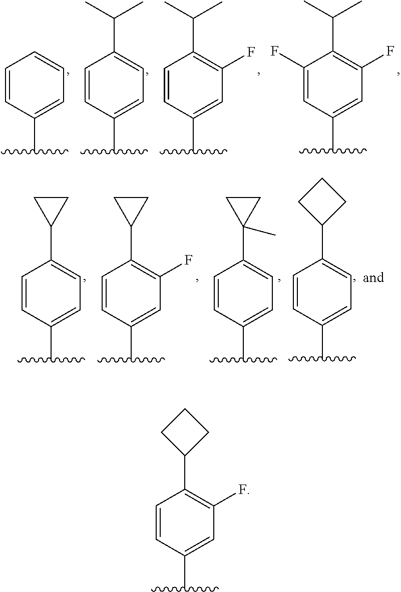

In some embodiments,

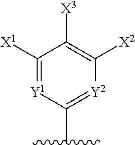

of formula (I) is selected from the group consisting of

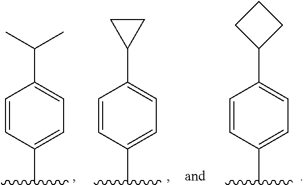

In some embodiments,

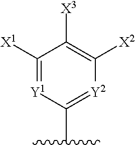

of formula (I) is

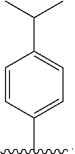 [SMILES: *c1ccc(C(C)C)cc1]

In some embodiments,

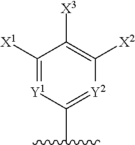

of formula (I) is

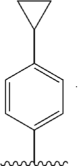 [SMILES: *c1ccc(C2CC2)cc1]

In some embodiments,

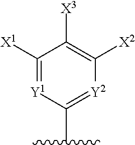

of formula (I) is

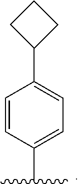 [SMILES: *c1ccc(C2CCC2)cc1]

In some embodiments of a compound of formula (I), or a stereoisomer or tautomer thereof, or a pharmaceutically acceptable salt of any of the foregoing, one of Y¹ and Y² is N and the other of Y¹ and Y² is CH.

In some embodiments of a compound of formula (I), or a stereoisomer or tautomer thereof, or a pharmaceutically acceptable salt of any of the foregoing,

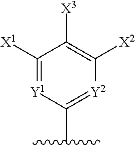

of formula (I) is selected from the group consisting of

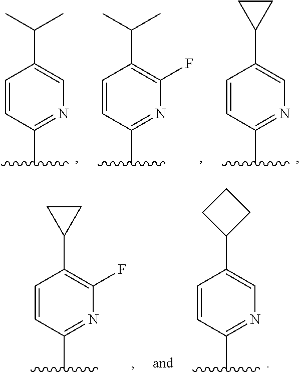

In some embodiments,

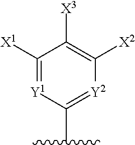

of formula (I) is selected from the group consisting of

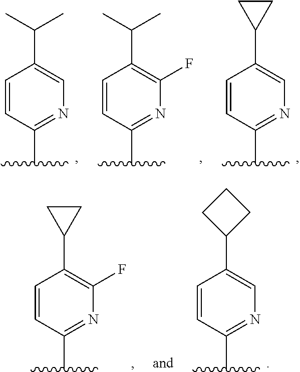

In some embodiments,

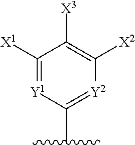

of formula (I) is selected from the group consisting of

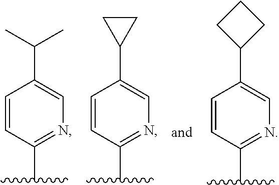

In some embodiments,

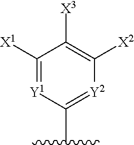

of formula (I) is

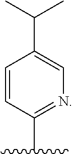 [SMILES: *c1ccc(C(C)C)cn1]

In some embodiments,

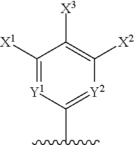

of formula (I) is

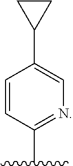 [SMILES: *c1ccc(C2CC2)cn1]

In some embodiments,

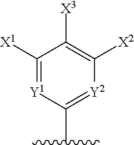

of formula (I) is

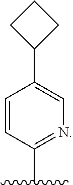 [SMILES: *c1ccc(C2CCC2)cn1]

In some embodiments of a compound of formula (I), or a stereoisomer or tautomer thereof, or a pharmaceutically acceptable salt of any of the foregoing, Q¹ is C₆₋₁₀cycloalkyl, wherein the C₆₋₁₀cycloalkyl of Q¹ is optionally substituted with one or more C₁₋₆alkyl. In some embodiments, C₆₋₈cycloalkyl, wherein the C₆₋₈cycloalkyl of Q¹ is optionally substituted with one or more C₁₋₃alkyl. In some embodiments, C₆₋₈cycloalkyl, wherein the C₆₋₈cycloalkyl of Q¹ is optionally substituted with one or more methyl. In some embodiments Q¹ is

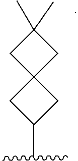 [SMILES: *C1CC2(C1)CC(C)(C)C2]

In some embodiments of a compound of formula (I), or a stereoisomer or tautomer thereof, or a pharmaceutically acceptable salt of any of the foregoing, X³ is H, and Q¹ is C₆₋₁₀cycloalkyl, wherein the C₆₋₁₀cycloalkyl of Q¹ is optionally substituted with one or more C₁₋₆alkyl. In some embodiments, X³ is H, and C₆₋₈cycloalkyl, wherein the C₆₋₈cycloalkyl of Q¹ is optionally substituted with one or more C₁₋₃alkyl.

In some embodiments of a compound of formula (I), or a stereoisomer or tautomer thereof, or a pharmaceutically acceptable salt of any of the foregoing, Q¹ is C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl. In some embodiments, Q¹ is C₆₋₁₀aryl, wherein the C₆₋₁₀aryl of Q¹ is optionally substituted with one or more R^{b}, wherein each R^{b} is independently C₁₋₄alkyl, —C₁₋₄alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₄alkyl. In some embodiments, Q¹ is C₆₋₁₀aryl, wherein the C₆₋₁₀aryl of Q¹ is optionally substituted with one or more R^{b}.

In some embodiments of a compound of formula (I), or a stereoisomer or tautomer thereof, or a pharmaceutically acceptable salt of any of the foregoing, each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl. In some embodiments, each R^{b} is independently C₁₋₄alkyl, —C₁₋₄alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₄alkyl. In some embodiments, each R^{b} is independently methyl, iso-propyl, sec-butyl, tert-butyl, methoxy, iso-propoxy, sec-butoxy, tert-butoxy, —NH—C(O)-(3-10 membered heterocyclyl), or 5-10 membered heteroaryl, wherein the 5-20 membered heteroaryl of R^{b} is optionally substituted with one or more C₁₋₄alkyl.

In some embodiments of a compound of formula (I), or a stereoisomer or tautomer thereof, or a pharmaceutically acceptable salt of any of the foregoing, Q¹ is phenyl, wherein the phenyl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl. In some embodiments, Q¹ is phenyl, wherein the phenyl of Q¹ is optionally substituted with one or more R^{b}, wherein each R^{b} is independently C₁₋₄alkyl, —C₁₋₄alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₄alkyl.

In some embodiments of a compound of formula (I), or a stereoisomer or tautomer thereof, or a pharmaceutically acceptable salt of any of the foregoing, Q¹ is selected from the group consisting of

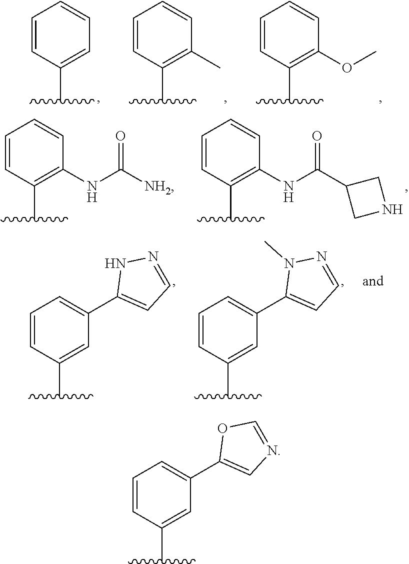

In some embodiments, Q¹ is selected from the group consisting of

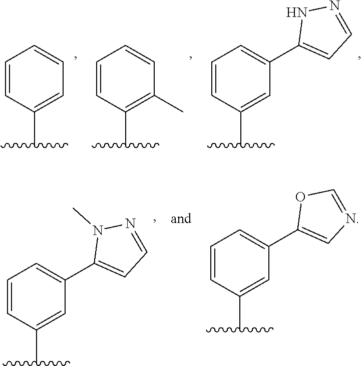

In some embodiments, Q¹ is selected from the group consisting of

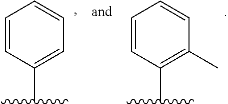

In some embodiments, Q¹ is

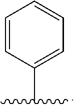 [SMILES: *c1ccccc1]

In some embodiments, Q¹ is

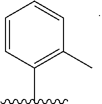 [SMILES: *c1ccccc1C]

In some embodiments of a compound of formula (I), or a stereoisomer or tautomer thereof, or a pharmaceutically acceptable salt of any of the foregoing, Q¹ is 3-15 membered heterocyclyl, wherein the 3-15 membered heterocyclyl of Q¹ is optionally substituted with one or more oxo or C₁₋₆alkyl. In some embodiments, Q¹ is 6-10 membered heterocyclyl, wherein the 6-10 membered heterocyclyl of Q¹ is optionally substituted with one or more oxo or C₁₋₃alkyl. In some embodiments, Q¹ is selected from the group consisting of

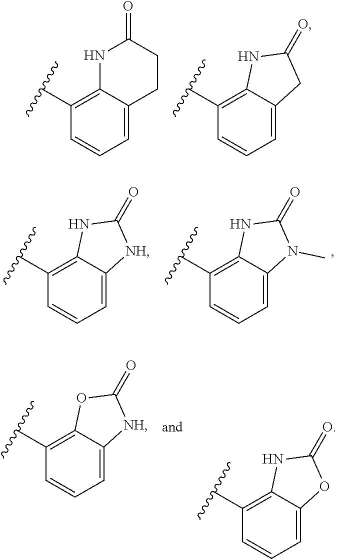

In some embodiments of a compound of formula (I), or a stereoisomer or tautomer thereof, or a pharmaceutically acceptable salt of any of the foregoing, Q¹ is 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1. In some embodiments, Q¹ is 5-10 membered heteroaryl, wherein the 5-10 membered heteroaryl of Q¹ is optionally substituted with one or more-NH₂, and wherein the 5-10 membered heteroaryl of Q¹ contains at least 1 annular N when m is 1. In some embodiments, Q¹ is pyridinyl, wherein the pyridinyl of Q¹ is optionally substituted with one or more-NH₂. In some embodiments, Q¹ is pyridinyl. In some embodiments, Q¹ is thiophenyl. In some embodiments, Q¹ is pyrazolyl.

In some embodiments of a compound of formula (I), or a stereoisomer or tautomer thereof, or a pharmaceutically acceptable salt of any of the foregoing, Q¹ is selected from the group consisting of

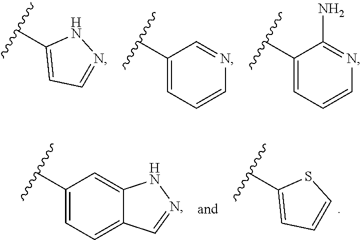

In some embodiments, Q¹ is selected from the group consisting of

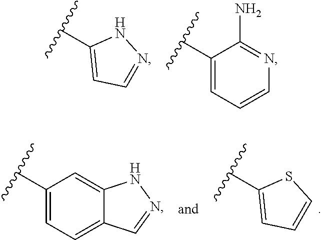

In some embodiments of a compound of formula (I), or a stereoisomer or tautomer thereof, or a pharmaceutically acceptable salt of any of the foregoing, R^{a} is H, halo, —OH, or —NH—C(O)—C₁₋₆alkoxy. In some embodiments, R^{a} is H, halo, —OH, or —NH—C(O)—C₁₋₃alkoxy. In some embodiments, R^{a} is H. In some embodiments, R^{a} is halo. In some embodiments, R^{a} is F. In some embodiments, R^{a} is —OH. In some embodiments, R^{a} is —NH—C(O)—C₁₋₃alkoxy. In some embodiments, R^{a} is —OH. In some embodiments, R^{a} is —NH—C(O)-tert-butoxy.

In some embodiments of a compound of formula (I), or any embodiment or variation thereof, such as a compound of formula (I-A), (I-A1), (I-A2), (I-B), (I-B1), (I-B2), (I-B3), (I-B4), (I-B5), (I-C), (I-D), (I-E), (I-E1), (I-E2), (I-F), (I-F1), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing, has a stereochemical configuration of the formula

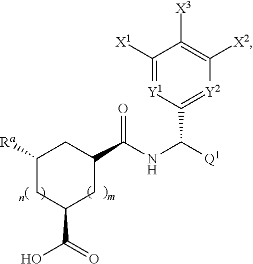

wherein m, n, X¹, X², X³, Y¹, Y², and R^{a}, are as defined elsewhere herein.

In some embodiments of a compound of formula (I), or any embodiment or variation thereof, such as a compound of formula (I-A), (I-A1), (I-A2), (I-B), (I-B1), (I-B2), (I-B3), (I-B4), (I-B5), (I-C), (I-D), (I-E), (I-E1), (I-E2), (I-F), (I-F1), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing, has a stereochemical configuration of the formula

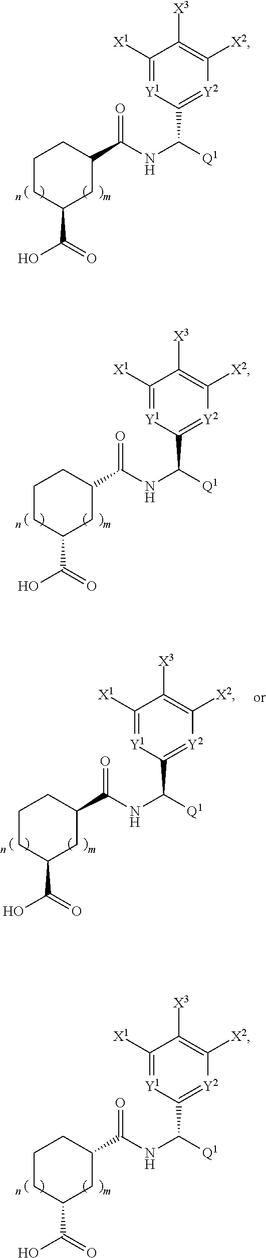

wherein m, n, X¹, X², X³, Y¹, and Y², are as defined elsewhere herein.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is phenyl wherein the phenyl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl; and R^{a} is H, halo, —OH, or —NH—C(O)—C₁₋₆alkoxy. In some embodiments, m is 0, or 1, and n is 0, 1, or 2, wherein m+n is an integer from 1 to 2; Y¹ and Y² are each CH, or one of Y¹ and Y² is N and the other of Y¹ and Y² is CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl wherein the phenyl of Q¹ is optionally substituted with one or more R^{b}, wherein each R^{b} is independently C₁₋₃alkyl, —C₁₋₃alkoxy, —NH—C(O)—NH₂, —NH—C(O)-(3-6 membered heterocyclyl), or 5-10 membered heteroaryl, wherein the 5-10 membered heteroaryl of R^{b} is optionally substituted with one or more C₁₋₃alkyl; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0; n is 1; Y¹ and Y² are each CH; X¹ and X² are each independently H; X³ is C₁₋₃alkyl, or C₃₋₆cycloalkyl; Q¹ is phenyl wherein the phenyl of Q¹ is optionally substituted with one or more R^{b}, wherein each R^{b} is independently C₁₋₃alkyl, —C₁₋₃alkoxy, or —NH—C(O)-(3-6 membered heterocyclyl); and R^{a} is H. In some embodiments, m is 0; n is 1; Y¹ and Y² are each CH; X¹ and X² are each independently H; X³ is methyl, or cyclopropyl; Q¹ is phenyl wherein the phenyl of Q¹ is optionally substituted with one or more R^{b}, wherein each R^{b} is independently methyl, methoxy, or —NH—C(O)— azetidinyl; and R^{a} is H.

In some embodiments of a compound of formula (I), or a stereoisomer or tautomer thereof, or a pharmaceutically acceptable salt of any of the foregoing, m is 0; n is 1; Y¹ and Y² are each CH; X¹ is H; X² is halo; X³ is C₁₋₃alkyl; Q¹ is phenyl wherein the phenyl of Q¹ is optionally substituted with one or more R^{b}, wherein each R^{b} is independently C₁₋₃alkyl, or —NH—C(O)—NH₂; and R^{a} is H. In some embodiments, m is 0; n is 1; Y¹ and Y² are each CH; X¹ is H; X² is F; X³ is isopropyl; Q¹ is phenyl wherein the phenyl of Q¹ is optionally substituted with one or more R^{b}, wherein each R^{b} is independently methyl, or —NH—C(O)—NH₂; and R^{a} is H.

In some embodiments of a compound of formula (I), or a stereoisomer or tautomer thereof, or a pharmaceutically acceptable salt of any of the foregoing, m is 0; and n is 1; Y¹ is CH; Y² is N; X¹ and X² are each independently H; X³ is C₁₋₃alkyl; Q¹ is phenyl wherein the phenyl of Q¹ is optionally substituted with one or more R^{b}, wherein each R^{b} is independently C₁₋₃alkyl; and R^{a} is H. In some embodiments, m is 0; and n is 1; Y¹ is CH; Y² is N; X¹ and X² are each independently H; X³ is isopropyl; Q¹ is phenyl wherein the phenyl of Q¹ is optionally substituted with one or more R^{b}, wherein each R^{b} is independently methyl; and R^{a} is H.

In some embodiments of a compound of formula (I), or a stereoisomer or tautomer thereof, or a pharmaceutically acceptable salt of any of the foregoing, m is 0; and n is 1; Y¹ is CH; Y² is N; X¹ is H; X² is halo; X³ is C₁₋₃alkyl; Q¹ is phenyl wherein the phenyl of Q¹ is optionally substituted with one or more R^{b}, wherein each R^{b} is independently 5-10 membered heteroaryl, wherein the 5-10 membered heteroaryl of R^{b} is optionally substituted with one or more C₁₋₃alkyl; and R^{a} is H. In some embodiments, m is 0; and n is 1; Y¹ is CH; Y² is N; X¹ is H; X² is F; X³ is isopropyl; Q¹ is phenyl wherein the phenyl of Q¹ is optionally substituted with one or more R^{b}, wherein each R^{b} is independently oxazolyl or pyrazolyl, wherein the oxazolyl or pyrazolyl of R^{b} is optionally substituted with one or more methyl; and R^{a} is H.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; Y¹ and Y² are each CH or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is phenyl; and R^{a} is H, halo, —OH, or —NH—C(O)—C₁₋₆alkoxy. In some embodiments, m is 0, or 1, and n is 0, 1, or 2, wherein m+n is an integer from 1 to 2; Y¹ and Y² are each CH or one of Y¹ and Y² is N and the other of Y¹ and Y² is CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; Y¹ and Y² are each CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy. In some embodiments, m is 0; n is 1; Y¹ and Y² are each CH; X¹ and X² are each independently halo; X³ is H, C₁₋₃alkyl; Q¹ is phenyl; and R^{a} is H. In some embodiments, m is 0; n is 1; Y¹ and Y² are each CH; X¹ and X² are each independently F; X³ is isopropyl; Q¹ is phenyl; and R^{a} is H.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; Y¹ and Y² are each CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy. In some embodiments, m is 0, or 1, and n is 0, 1, or 2, wherein m+n is an integer from 1 to 2; Y¹ and Y² are each CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0; n is 1; Y¹ and Y² are each CH; X¹ is H; X² is halo; X³ is C₁₋₃alkyl, or C₃₋₆cycloalkyl; Q¹ is phenyl; and R^{a} is H. In some embodiments, m is 0; n is 1; Y¹ and Y² are each CH; X¹ is H; X² is halo; X³ is isopropyl, cyclopropyl, or cyclobutyl; Q¹ is phenyl; and R^{a} is H.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; one of Y¹ and Y² is N and the other of Y¹ and Y² is CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy. In some embodiments, m is 0, or 1, and n is 0, 1, or 2, wherein m+n is an integer from 1 to 2; one of Y¹ and Y² is N and the other of Y¹ and Y² is CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; one of Y¹ and Y² is N and the other of Y¹ and Y² is CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy. In some embodiments, m is 0, or 1, and n is 0, 1, or 2, wherein m+n is an integer from 1 to 2; one of Y¹ and Y² is N and the other of Y¹ and Y² is CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is phenyl; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0; n is 1; Y¹ is CH; Y² is N; X¹ is H; X² is halo; X³ is C₃₋₆cycloalkyl; Q¹ is phenyl; and R^{a} is H. In some embodiments, m is 0; n is 1; Y¹ is CH; Y² is N; X¹ is H; X² is halo; X³ is cyclopropyl; Q¹ is phenyl; and R^{a} is H.

In some embodiments of a compound of formula (I), or a stereoisomer or tautomer thereof, or a pharmaceutically acceptable salt of any of the foregoing, m is 0; n is 1; Y¹ is CH; Y² is N; X¹ and X² are each H; X³ is C₁₋₃alkyl, or C₃₋₆cycloalkyl; Q¹ is phenyl; and R^{a} is H. In some embodiments, m is 0; n is 1; Y¹ is CH; Y² is N; X¹ and X² are each H; X³ is isopropyl, cyclopropyl, or cyclobutyl; Q¹ is phenyl; and R^{a} is H.

In some embodiments of a compound of formula (I), or a stereoisomer or tautomer thereof, or a pharmaceutically acceptable salt of any of the foregoing, m is 0, or 1, and n is 0, 1, or 2, wherein m+n is an integer from 1 to 2; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) 3-15 membered heterocyclyl, wherein the 3-15 membered heterocyclyl of Q¹ is optionally substituted with one or more oxo or C₁₋₆alkyl, or (ii) 5-20 membered heteroaryl, wherein the 5-20 membered heteroaryl of Q¹ is optionally substituted with one or more —NH₂; and R^{a} is H, halo, —OH, or —NH—C(O)—C₁₋₆alkoxy. In some embodiments, m is 0, or 1, and n is 0, 1, or 2, wherein m+n is an integer from 1 to 2; Y¹ and Y² are each CH, or one of Y¹ and Y² is N and the other of Y¹ and Y² is CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is (i) 3-10 membered heterocyclyl, wherein the 3-10 membered heterocyclyl of Q¹ is optionally substituted with one or more oxo or C₁₋₃alkyl, or (ii) 5-10 membered heteroaryl, wherein the 5-10 membered heteroaryl of Q¹ is optionally substituted with one or more —NH₂; and R^{a} is H, halo, —OH, or —NH—C(O)—C₁₋₃alkoxy.

In some embodiments of a compound of formula (I), or a stereoisomer or tautomer thereof, or a pharmaceutically acceptable salt of any of the foregoing, m is 0; n is 1; Y¹ and Y² are each CH; X¹ and X² are each independently H; X³ is C₁₋₆alkyl; Q¹ is 3-15 membered heterocyclyl, wherein the 3-15 membered heterocyclyl of Q¹ is optionally substituted with one or more oxo or C₁₋₆alkyl; and R^{a} is H. In some embodiments, m is 0; n is 1; Y¹ and Y² are each CH; X¹ and X² are each independently H; X³ is C₁₋₃alkyl; Q¹ is 3-10 membered heterocyclyl, wherein the 3-10 membered heterocyclyl of Q¹ is optionally substituted with one or more oxo or C₁₋₃alkyl; and R^{a} is H. In some embodiments, m is 0; n is 1; Y¹ and Y² are each CH; X¹ and X² are each independently H; X³ is C₁₋₃alkyl; Q¹ is indolinyl, dihydro-2H-benzo[d]imidazolyl, benzo[d]oxazolyl, or 3,4-dihydroquinolinyl, wherein the indolinyl, dihydro-2H-benzo[d]imidazolyl, benzo[d]oxazolyl, or 3,4-dihydroquinolinyl of Q¹ is optionally substituted with one or more oxo; and R^{a} is H.

In some embodiments of a compound of formula (I), or a stereoisomer or tautomer thereof, or a pharmaceutically acceptable salt of any of the foregoing, m is 0; n is 1; Y¹ is CH; Y² is N; X¹ is H; X² is halo; X³ is C₁₋₆alkyl; Q¹ is 3-15 membered heterocyclyl, wherein the 3-15 membered heterocyclyl of Q¹ is optionally substituted with one or more oxo; and R^{a} is H. In some embodiments, m is 0; n is 1; Y¹ is CH; Y² is N; X¹ is H; X² is halo; X³ is C₁₋₃alkyl; Q¹ is 3-10 membered heterocyclyl, wherein the 3-10 membered heterocyclyl of Q¹ is optionally substituted with one or more oxo; and R^{a} is H. In some embodiments, m is 0; n is 1; Y¹ is CH; Y² is N; X¹ is H; X² is F; X³ is C₁₋₃alkyl; Q¹ is dihydro-2H-benzo[d]imidazolyl, benzo[d]oxazolyl, wherein the dihydro-2H-benzo[d]imidazolyl of Q¹ is optionally substituted with one or more oxo or methyl; and R^{a} is H.

In some embodiments of a compound of formula (I), or a stereoisomer or tautomer thereof, or a pharmaceutically acceptable salt of any of the foregoing, m is 0; n is 1; Y¹ and Y² are each CH; X¹ is H; X² is halo; X³ is C₁₋₆alkyl; Q¹ is 5-20 membered heteroaryl; and R^{a} is H. In some embodiments, m is 0; n is 1; Y¹ and Y² are each CH; X¹ is H; X² is halo; X³ is C₁₋₃alkyl; Q¹ is 5-20 membered heteroaryl; and R^{a} is H. In some embodiments, m is 0; n is 1; Y¹ and Y² are each CH; X¹ is H; X² is F; X³ is C₁₋₃alkyl; Q¹ is 1H-benzo[d]imidazole; and R^{a} is H.

In some embodiments of a compound of formula (I), or a stereoisomer or tautomer thereof, or a pharmaceutically acceptable salt of any of the foregoing, m is 0; n is 1, or 2; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy. In some embodiments, m is 0; n is 1; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0; n is 2; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0; n is 2; Y¹ and Y² are each CH, or one of Y¹ and Y² is N and the other of Y¹ and Y² is CH; X¹ and X² are each independently H; X³ is C₁₋₆alkyl; Q¹ is 5-20 membered heteroaryl; and R^{a} is H. In some embodiments, m is 0; n is 2; Y¹ and Y² are each CH, or one of Y¹ and Y² is N and the other of Y¹ and Y² is CH; X¹ and X² are each independently H; X³ is C₁₋₃alkyl; Q¹ is 5-10 membered heteroaryl; and R^{a} is H. In some embodiments, m is 0; n is 2; Y¹ and Y² are each CH, or one of Y¹ and Y² is N and the other of Y¹ and Y² is CH; X¹ and X² are each independently H; X³ is isopropyl; Q¹ is thiophenyl; and R^{a} is H.

In some embodiments of a compound of formula (I), or a stereoisomer or tautomer thereof, or a pharmaceutically acceptable salt of any of the foregoing, m is 1; n is 0, or 1; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 1; n is 0; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 1; n is 0; Y¹ and Y² are each CH; X¹ and X² are each independently H; X³ is C₁₋₆alkyl; Q¹ is C₆₋₂₀aryl; and R^{a} is H. In some embodiments, m is 1; n is 0; Y¹ and Y² are each CH; X¹ and X² are each independently H; X³ is C₁₋₃alkyl; Q¹ is C₆₋₁₀aryl; and R^{a} is H. In some embodiments, m is 1; n is 0; Y¹ and Y² are each CH; X¹ and X² are each independently H; X³ is isopropyll; Q¹ is phenyl; and R^{a} is H.

In some embodiments of a compound of formula (I), or a stereoisomer or tautomer thereof, or a pharmaceutically acceptable salt of any of the foregoing, m is 1; n is 1; Y¹ and Y² are each CH, or one of Y¹ and Y² is N and the other of Y¹ and Y² is CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0; n is 1; Y¹ and Y² are each CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy. In some embodiments, m is 0; n is 1; Y¹ and Y² are each CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing m is 0, and n is 1, wherein m+n is an integer from 1 to 2; Y¹ and Y² are each CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy. In some embodiments, m is 0, and n is 1, wherein m+n is an integer from 1 to 2; Y¹ and Y² are each CH; X¹ and X² are each independently H or halo; X³ is C₁₋₃alkyl, or C₃₋₆cycloalkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0; n is 1; Y¹ and Y² are each CH; X¹ and X² are each independently H or halo; X³ is H, C₁₋₆alkyl, or C₃₋₁₀cycloalkyl, wherein the C₃₋₁₀cycloalkyl of X³ is optionally substituted with one or more C₁₋₆alkyl; Q¹ is (i) C₆₋₁₀cycloalkyl, wherein the C₆₋₁₀cycloalkyl of Q¹ is optionally substituted with one or more C₁₋₆alkyl, (ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl, (iii) 3-15 membered heterocyclyl, wherein the 3-15 membered heterocyclyl of Q¹ is optionally substituted with one or more oxo or C₁₋₆alkyl, or (iv) 5-20 membered heteroaryl, wherein the 5-20 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy. In some embodiments, m is 0; n is 1; Y¹ and Y² are each CH; X¹ and X² are each independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments of a compound of formula (I), or a stereoisomer or tautomer thereof, or a pharmaceutically acceptable salt of any of the foregoing, m is 0, and n is 1, wherein m+n is an integer from 1 to 2; one of Y¹ and Y² is N and the other of Y¹ and Y² is CH; one of X¹ and X² is H and the other of X¹ and X² is independently H or halo; X³ is H, C₁₋₃alkyl, or C₃₋₆cycloalkyl, wherein the C₃₋₆cycloalkyl of X³ is optionally substituted with one or more C₁₋₃alkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy. In some embodiments, m is 0, and n is 1, wherein m+n is an integer from 1 to 2; one of Y¹ and Y² is N and the other of Y¹ and Y² is CH; X¹ and X² are each independently H or halo; X³ is C₁₋₃alkyl, or C₃₋₆cycloalkyl; Q¹ is (i) C₆₋₈cycloalkyl, wherein the C₆₋₈cycloalkyl of Q¹ is optionally substituted with one or more C₁₋₃alkyl, (ii) C₆₋₁₉aryl, wherein the C₆₋₁₀aryl of Q¹ is optionally substituted with one or more R^{b}, wherein each R^{b} is independently C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl of R^{b} is optionally substituted with one or more C₁₋₆alkyl, (iii) 3-10 membered heterocyclyl, wherein the 3-10 membered heterocyclyl of Q¹ is optionally substituted with one or more oxo or C₁₋₃alkyl, or (iv) 5-10 membered heteroaryl, wherein the 5-10 membered heteroaryl of Q¹ is optionally substituted with one or more-NH₂, provided that, when X³ is H, then Q¹ is C₆₋₁₀cycloalkyl, wherein the C₆₋₁₀cycloalkyl of Q¹ is optionally substituted with one or more C₁₋₆alkyl; and R^{a} is H, halo, —OH, or —NH—C(O)—C₁₋₆alkoxy.

In some embodiments, provided herein is a compound of formula (I), or a stereoisomer or tautomer thereof, or a pharmaceutically acceptable salt thereof, wherein the compound is a compound of formula (I-A):

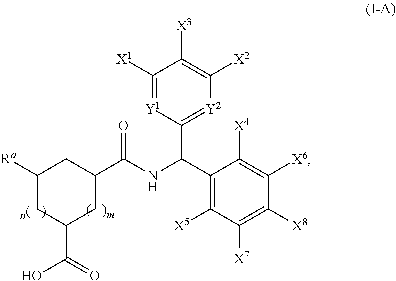

or a stereoisomer or tautomer thereof, or a pharmaceutically acceptable salt of any of the foregoing, wherein, either:
i. X⁴⁻⁸ are each independently H, C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl; or
ii. X⁶ is taken together with either of X⁴ or X⁸, and the atoms to which they are attached, to form ring A, wherein ring A is
   3-9 membered heterocyclyl, wherein the 3-9 membered heterocyclyl of ring A is optionally substituted with one or more oxo or C₁₋₆alkyl, and wherein X⁵, X⁷, and the other of X⁴ or X⁸ are each independently H, oxo or C₁₋₆alkyl, or
   5-14 membered heteroaryl, wherein the 5-14 membered heteroaryl of ring A is optionally substituted with one or more-NH₂, and wherein the 5-14 membered heteroaryl of ring A contains at least 1 annular N when m is 1, and wherein X⁵, X⁷, and the other of X⁴ or X⁸ are each independently H, or more-NH₂; or
iii. X⁷ is taken together with either of X⁵ or X⁸, and the atoms to which they are attached, to form ring A, wherein ring A is
   3-9 membered heterocyclyl, wherein the 3-9 membered heterocyclyl of ring A is optionally substituted with one or more oxo or C₁₋₆alkyl, and wherein X⁴, X⁶, and the other of X⁵ or X⁸ are each independently H, oxo or C₁₋₆alkyl, or
   5-14 membered heteroaryl, wherein the 5-14 membered heteroaryl of ring A is optionally substituted with one or more-NH₂, and wherein the 5-14 membered heteroaryl of ring A contains at least 1 annular N when m is 1, and wherein X⁴, X⁶, and the other of X⁵ or X⁸ are each independently H, or —NH₂.

In some embodiments of a compound of formula (I-A), or a stereoisomer or tautomer thereof, or a pharmaceutically acceptable salt of any of the foregoing, X⁴⁻⁸ are each independently H, C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl. In some embodiments, X⁴⁻⁸ are each independently H, C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl is independently optionally substituted with one or more C₁₋₃alkyl.

In some embodiments of a compound of formula (I-A), or a stereoisomer or tautomer thereof, or a pharmaceutically acceptable salt of any of the foregoing, X⁴⁻⁸ are each independently H.

In some embodiments of a compound of formula (I-A), or a stereoisomer or tautomer thereof, or a pharmaceutically acceptable salt of any of the foregoing, one of X⁴⁻⁸ is C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl and the others of X⁴⁻⁸ are each independently H. In some embodiments, one of X⁴⁻⁸ is C₁₋₃alkyl, —C₁₋₃lkoxy, —NH—C(O)—NH₂, —NH—C(O)-(3-10 membered heterocyclyl), or 5-10 membered heteroaryl, wherein the 5-10 membered heteroaryl is independently optionally substituted with one or more C₁₋₃alkyl and the others of X⁴⁻⁸ are each independently H. In some embodiments, one of X⁴⁻⁸ is selected from the group consisting of methyl, —OCH₃,

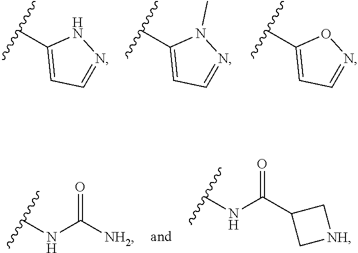

and the others of X⁴⁻⁸ are each independently H.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is 3-9 membered heterocyclyl, wherein the 3-9 membered heterocyclyl of ring A is optionally substituted with one or more oxo or C₁₋₆alkyl, and wherein X⁵, X⁷, and the other of X⁴ or X⁸ are each independently H, oxo or C₁₋₆alkyl, or
5-14 membered heteroaryl, wherein the 5-14 membered heteroaryl of ring A is optionally substituted with one or more-NH₂, and wherein the 5-14 membered heteroaryl of ring A contains at least 1 annular N when m is 1, and wherein X⁵, X⁷, and the other of X⁴ or X⁸ are each independently H, or more-NH₂.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is 3-9 membered heterocyclyl, wherein the 3-9 membered heterocyclyl of ring A is optionally substituted with one or more oxo or C₁₋₆alkyl, and wherein X⁴, X⁶, and the other of X⁵ or X⁸ are each independently H, oxo or C₁₋₆alkyl, or
5-14 membered heteroaryl, wherein the 5-14 membered heteroaryl of ring A is optionally substituted with one or more-NH₂, and wherein the 5-14 membered heteroaryl of ring A contains at least 1 annular N when m is 1, and wherein X⁴, X⁶, and the other of X⁵ or X⁸ are each independently H, or —NH₂.

In some embodiments of a compound of formula (I-A), or a stereoisomer or tautomer thereof, or a pharmaceutically acceptable salt of any of the foregoing, one of X⁴⁻⁸ is C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl and the others of X⁴⁻⁸ are each independently H. In some embodiments, one of X⁴⁻⁸ is C₁₋₃alkyl, —C₁₋₃lkoxy, —NH—C(O)—NH₂, —NH—C(O)-(3-10 membered heterocyclyl), or 5-10 membered heteroaryl, wherein the 5-10 membered heteroaryl is independently optionally substituted with one or more C₁₋₃alkyl and the others of X⁴⁻⁸ are each independently H. In some embodiments, one of X⁴⁻⁸ is selected from the group consisting of methyl, —OCH₃,

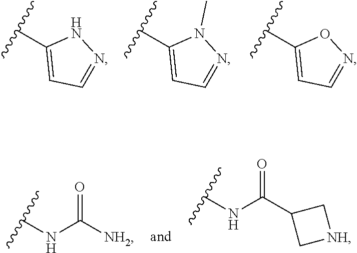

and the others of X⁴⁻⁸ are each independently H.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is 3-9 membered heterocyclyl, wherein the 3-9 membered heterocyclyl of ring A is optionally substituted with one or more oxo or C₁₋₆alkyl. In some embodiments, ring A is 5-6 membered heterocyclyl, wherein the 5-6 membered heterocyclyl of ring A is optionally substituted with one or more oxo or C₁₋₃alkyl. In some embodiments, ring A is selected from the group consisting of

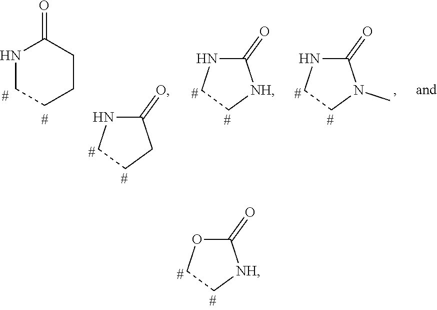

wherein # represents a point of attachment to the rest of the molecule.

In some embodiments of a compound of formula (I-A), or a stereoisomer or tautomer thereof, or a pharmaceutically acceptable salt of any of the foregoing, X⁶ is taken together with either of X⁴ or X⁸, and the atoms to which they are attached, to form ring A, wherein ring A is 5-8 membered heteroaryl, wherein the 5-8 membered heteroaryl of ring A is optionally substituted with one or more-NH₂, and wherein the 5-8 membered heteroaryl of ring A contains at least 1 annular N when m is 1. In some embodiments, ring A is 5-8 membered heteroaryl, wherein the 5-8 membered heteroaryl of ring A is optionally substituted with one or more-NH₂, and wherein the 5-8 membered heteroaryl of ring A contains at least 1 annular N when m is 1. In some embodiments, ring A is

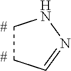

wherein # represents a point of attachment to the rest of the molecule.

In some embodiments, provided herein is a compound of formula (I), or (I-A), or a stereoisomer or tautomer thereof, or a pharmaceutically acceptable salt thereof, wherein the compound is a compound of formula (I-A1):

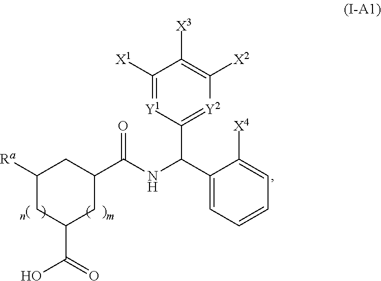

or a stereoisomer or tautomer thereof, or a pharmaceutically acceptable salt of any of the foregoing, wherein X⁴ is H, C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl. In some embodiments, X⁴ is H, C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl is independently optionally substituted with one or more C₁₋₃alkyl.

In some embodiments, provided herein is a compound of formula (I), or (I-A), or a stereoisomer or tautomer thereof, or a pharmaceutically acceptable salt thereof, wherein the compound is a compound of formula (I-A2):

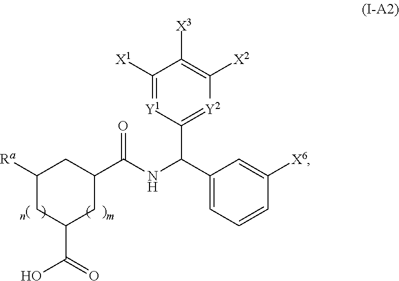

or a stereoisomer or tautomer thereof, or a pharmaceutically acceptable salt of any of the foregoing, wherein X⁶ is H, C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl. In some embodiments, X⁶ is H, C₁₋₃alkyl, —C₁₋₃alkoxy, —NH—C(O)—NH₂, —NH—C(O)-(3-10 membered heterocyclyl), or 5-10 membered heteroaryl, wherein the 5-10 membered heteroaryl is independently optionally substituted with one or more C₁₋₃alkyl.

In some embodiments, provided herein is a compound of formula (I), or a stereoisomer or tautomer thereof, or a pharmaceutically acceptable salt thereof, wherein the compound is a compound of formula (I-B):

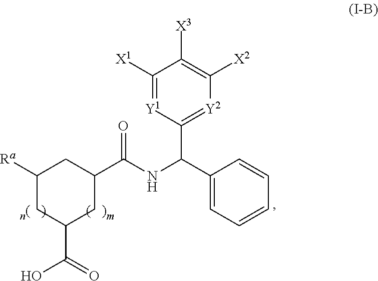

or a stereoisomer or tautomer thereof, or a pharmaceutically acceptable salt of any of the foregoing.

In some embodiments of a compound of formula (I-B), or a stereoisomer or tautomer thereof, or a pharmaceutically acceptable salt of any of the foregoing, X¹ and X² are independently H or halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, X¹ and X² are independently H or F; and X³ is H, C₁₋₃alkyl, or C₃₋₆cycloalkyl, wherein the C₃₋₆cycloalkyl of X³ is optionally substituted with one or more C₁₋₃alkyl. In some embodiments, one of X¹ and X² is H; the other of X¹ and X² is halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, one of X¹ and X² is H; the other of X¹ and X² is F; and X³ is H, C₁₋₃alkyl, or C₃₋₆cycloalkyl, wherein the C₃₋₆cycloalkyl of X³ is optionally substituted with one or more C₁₋₃alkyl.

In some embodiments of a compound of formula (I-B), or a stereoisomer or tautomer thereof, or a pharmaceutically acceptable salt of any of the foregoing, Y¹ and Y² are each CH; X¹ and X² are independently H or halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, Y¹ and Y² are each CH; X¹ and X² are independently H or F; and X³ is H, C₁₋₃alkyl, or C₃₋₆cycloalkyl, wherein the C₃₋₆cycloalkyl of X³ is optionally substituted with one or more C₁₋₃alkyl. In some embodiments, Y¹ and Y² are each CH; one of X¹ and X² is H; the other of X¹ and X² is halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, Y¹ and Y² are each CH; one of X¹ and X² is H; the other of X¹ and X² is F; and X³ is H, C₁₋₃alkyl, or C₃₋₆cycloalkyl, wherein the C₃₋₆cycloalkyl of X³ is optionally substituted with one or more C₁₋₃alkyl.

In some embodiments of a compound of formula (I-B), or a stereoisomer or tautomer thereof, or a pharmaceutically acceptable salt of any of the foregoing, one of Y¹ and Y² is N and the other of Y¹ and Y² is CH; X¹ and X² are independently H or halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, one of Y¹ and Y² is N and the other of Y¹ and Y² is CH; X¹ and X² are independently H or F; and X³ is H, C₁₋₃alkyl, or C₃₋₆cycloalkyl, wherein the C₃₋₆cycloalkyl of X³ is optionally substituted with one or more C₁₋₃alkyl. In some embodiments, one of Y¹ and Y² is N and the other of Y¹ and Y² is CH; one of X¹ and X² is H; the other of X¹ and X² is halo; and X³ is H, C₁₋₆alkyl, or C₃₋₁₀cycloalkyl, wherein the C₃₋₁₀cycloalkyl of X³ is optionally substituted with one or more C₁₋₆alkyl. In some embodiments, one of Y¹ and Y² is N and the other of Y¹ and Y² is CH, one of X¹ and X² is H; the other of X¹ and X² is F; and X³ is H, C₁₋₃alkyl, or C₃₋₆cycloalkyl, wherein the C₃₋₆cycloalkyl of X³ is optionally substituted with one or more C₁₋₃alkyl.

In some embodiments, provided herein is a compound of formula (I) or formula (I-B), or a stereoisomer or tautomer thereof, or a pharmaceutically acceptable salt thereof, wherein the compound is of formula (I-B1):

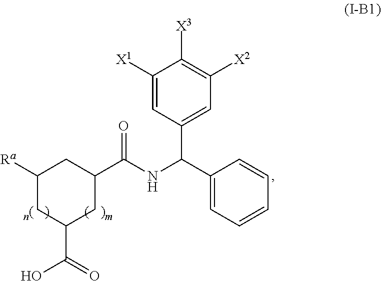

or a stereoisomer or tautomer thereof, or a pharmaceutically acceptable salt of any of the foregoing.

In some embodiments of a compound of formula (I-B1), or a stereoisomer or tautomer thereof, or a pharmaceutically acceptable salt of any of the foregoing, X¹ and X² are independently halo; and X³ is C₁₋₆alkyl, or C₃₋₁₀cycloalkyl, wherein the C₃₋₁₀cycloalkyl of X³ is optionally substituted with one or more C₁₋₆alkyl. In some embodiments, X¹ and X² are independently F; and X³ is C₁₋₃alkyl, or C₃₋₆cycloalkyl, wherein the C₃₋₆cycloalkyl of X³ is optionally substituted with one or more C₁₋₃alkyl.

In some embodiments, provided is a compound of formula (I) or formula (I-B), or a stereoisomer or tautomer thereof, or a pharmaceutically acceptable salt of any of the foregoing, wherein the compound is a compound of formula (I-B2):

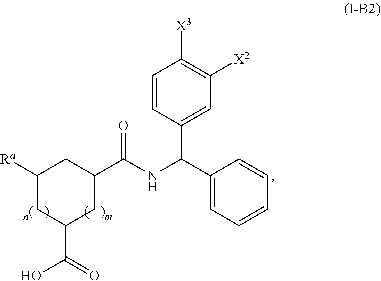

or a stereoisomer or tautomer thereof, or a pharmaceutically acceptable salt of any of the foregoing.

In some embodiments of a compound of formula (I-B2), or a stereoisomer or tautomer thereof, or a pharmaceutically acceptable salt of any of the foregoing, X² is halo; and X³ is C₁₋₆alkyl, or C₃₋₁₀cycloalkyl, wherein the C₃₋₁₀cycloalkyl of X³ is optionally substituted with one or more C₁₋₆alkyl. In some embodiments, X² is F; and X³ is C₁₋₃alkyl, or C₃₋₆cycloalkyl, wherein the C₃₋₆cycloalkyl of X³ is optionally substituted with one or more C₁₋₃alkyl.

In some embodiments, provided is a compound of formula (I) or formula (I-B), or a stereoisomer or tautomer thereof, or a pharmaceutically acceptable salt of any of the foregoing, wherein the compound is a compound of formula (I-B3):

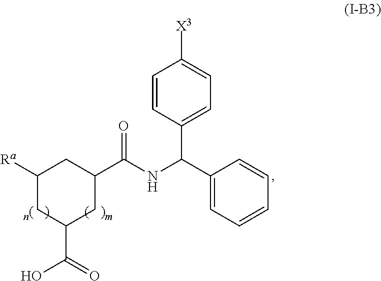

or a stereoisomer or tautomer thereof, or a pharmaceutically acceptable salt of any of the foregoing.

In some embodiments of a compound of formula (I-B3), or a stereoisomer or tautomer thereof, or a pharmaceutically acceptable salt of any of the foregoing, X³ is C₁₋₆alkyl, or C₃₋₁₀cycloalkyl, wherein the C₃₋₁₀cycloalkyl of X³ is optionally substituted with one or more C₁₋₆alkyl. In some embodiments, X³ is C₁₋₃alkyl, or C₃₋₆cycloalkyl, wherein the C₃₋₆cycloalkyl of X³ is optionally substituted with one or more C₁₋₃alkyl.

In some embodiments, provided herein is a compound of formula (I) or formula (I-B), or a stereoisomer or tautomer thereof, or a pharmaceutically acceptable salt thereof, wherein the compound is of formula (I-B4):

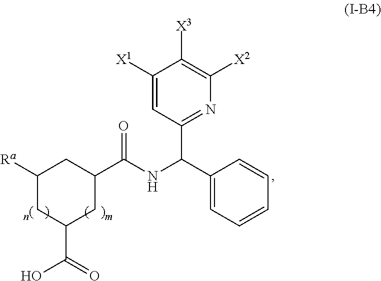

or a stereoisomer or tautomer thereof, or a pharmaceutically acceptable salt of any of the foregoing.

In some embodiments, provided is a compound of formula (I) or formula (I-B), or a stereoisomer or tautomer thereof, or a pharmaceutically acceptable salt of any of the foregoing, wherein the compound is a compound of formula (I-B5):

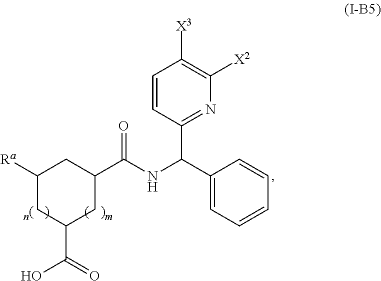

or a stereoisomer or tautomer thereof, or a pharmaceutically acceptable salt of any of the foregoing.

In some embodiments of a compound of formula (I-B5), or a stereoisomer or tautomer thereof, or a pharmaceutically acceptable salt of any of the foregoing, X² is halo; and X³ is C₁₋₆alkyl, or C₃₋₁₀cycloalkyl, wherein the C₃₋₁₀cycloalkyl of X³ is optionally substituted with one or more C₁₋₆alkyl. In some embodiments, X² is F; and X³ is C₁₋₃alkyl, or C₃₋₆cycloalkyl, wherein the C₃₋₆cycloalkyl of X³ is optionally substituted with one or more C₁₋₃alkyl.

In some embodiments, provided is a compound of formula (I) or formula (I-B), or a stereoisomer or tautomer thereof, or a pharmaceutically acceptable salt of any of the foregoing, wherein the compound is a compound of formula (I-B6):

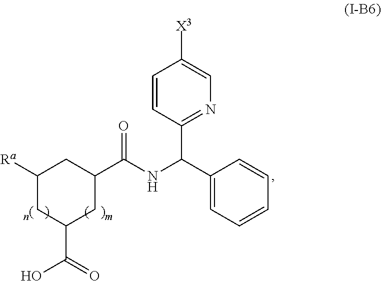

or a stereoisomer or tautomer thereof, or a pharmaceutically acceptable salt of any of the foregoing.

In some embodiments of a compound of formula (I-B6), or a stereoisomer or tautomer thereof, or a pharmaceutically acceptable salt of any of the foregoing, X³ is C₁₋₆alkyl, or C₃₋₁₀cycloalkyl, wherein the C₃₋₁₀cycloalkyl of X³ is optionally substituted with one or more C₁₋₆alkyl. In some embodiments, X³ is C₁₋₃alkyl, or C₃₋₆cycloalkyl, wherein the C₃₋₆cycloalkyl of X³ is optionally substituted with one or more C₁₋₃alkyl.

In some embodiments, provided here is a compound of formula (I), or a stereoisomer or tautomer thereof, or a pharmaceutically acceptable salt of any of the foregoing, wherein the compound is a compound of formula (I-C):

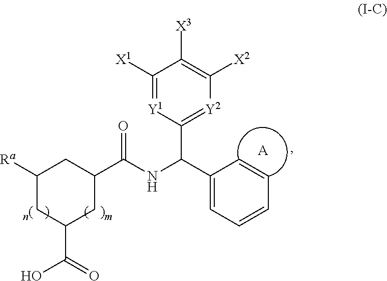

or a stereoisomer or tautomer thereof, or a pharmaceutically acceptable salt of any of the foregoing, wherein ring A is
3-9 membered heterocyclyl, wherein the 3-9 membered heterocyclyl of ring A is optionally substituted with one or more oxo or C₁₋₆alkyl, or
5-14 membered heteroaryl, wherein the 5-14 membered heteroaryl of ring A is optionally substituted with one or more-NH₂, and wherein the 5-14 membered heteroaryl of ring A contains at least 1 annular N when m is 1.

In some embodiments, provided here is a compound of formula (I), or a stereoisomer or tautomer thereof, or a pharmaceutically acceptable salt of any of the foregoing, wherein the compound is a compound of formula (I-D):

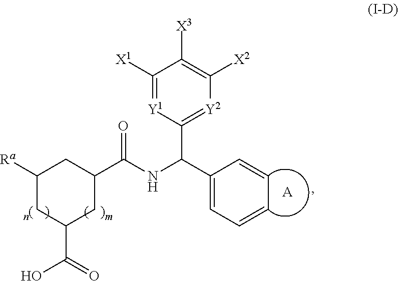

or a stereoisomer or tautomer thereof, or a pharmaceutically acceptable salt of any of the foregoing, wherein ring A is
3-9 membered heterocyclyl, wherein the 3-9 membered heterocyclyl of ring A is optionally substituted with one or more oxo or C₁₋₆alkyl, or
5-14 membered heteroaryl, wherein the 5-14 membered heteroaryl of ring A is optionally substituted with one or more-NH₂, and wherein the 5-14 membered heteroaryl of ring A contains at least 1 annular N when m is 1.

In some embodiments of a compound of formula (I-C), (I-D), or a stereoisomer or tautomer thereof, or a pharmaceutically acceptable salt of any of the foregoing, ring A is 5-6 membered heterocyclyl, wherein the 5-6 membered heterocyclyl of ring A is optionally substituted with one or more oxo or C₁₋₃alkyl. In some embodiments, ring A is selected from the group consisting of

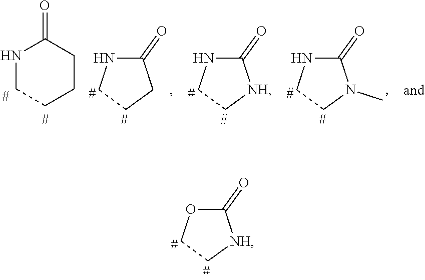

wherein # represents a point of attachment to the rest of the molecule.

In some embodiments of a compound of formula (I-C), or (I-D), or a stereoisomer or tautomer thereof, or a pharmaceutically acceptable salt of any of the foregoing, ring A is 5-8 membered heteroaryl, wherein the 5-8 membered heteroaryl of ring A is optionally substituted with one or more —NH₂, and wherein the 5-8 membered heteroaryl of ring A contains at least 1 annular N when m is 1. In some embodiments, ring A is 5-8 membered heteroaryl, wherein the 5-8 membered heteroaryl of ring A is optionally substituted with one or more-NH₂, and wherein the 5-8 membered heteroaryl of ring A contains at least 1 annular N when m is 1. In some embodiments, ring A is

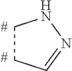

wherein # represents a point of attachment to the rest of the molecule.

In some embodiments, provided herein is a compound of formula (I), or a stereoisomer or tautomer thereof, or a pharmaceutically acceptable salt of any of the foregoing, wherein the compound is a compound of formula (I-E):

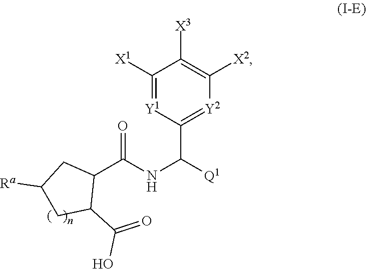

or a stereoisomer or tautomer thereof, or a pharmaceutically acceptable salt of any of the foregoing.

In some embodiments, provided herein is a compound of formula (I), or (I-E), or a stereoisomer or tautomer thereof, or a pharmaceutically acceptable salt of any of the foregoing, wherein the compound is a compound of formula (I-E1):

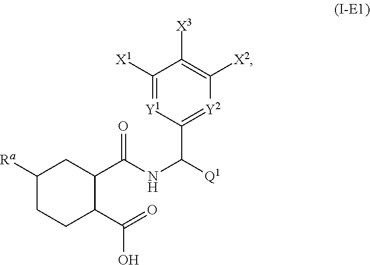

or a stereoisomer or tautomer thereof, or a pharmaceutically acceptable salt of any of the foregoing.

In some embodiments, provided herein is a compound of formula (I), or (I-E), or a stereoisomer or tautomer thereof, or a pharmaceutically acceptable salt of any of the foregoing, wherein the compound is a compound of formula (I-E2):

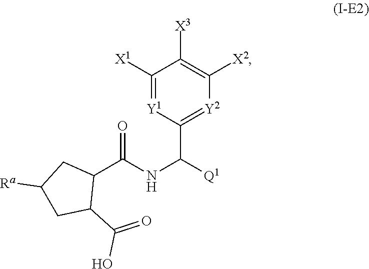

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is a compound of formula (I-F):

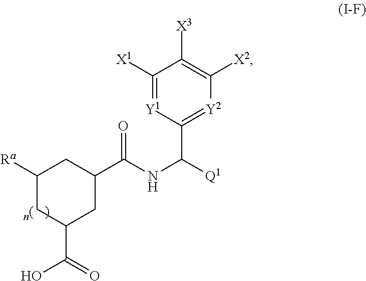

or a stereoisomer or tautomer thereof, or a pharmaceutically acceptable salt of any of the foregoing.

In some embodiments, provided herein is a compound of formula (I), or (I-F), or a stereoisomer or tautomer thereof, or a pharmaceutically acceptable salt of any of the foregoing, wherein the compound is a compound of formula (I-F1):

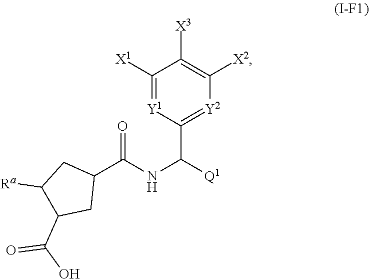

or a stereoisomer or tautomer thereof, or a pharmaceutically acceptable salt of any of the foregoing.

In some embodiments, provided herein is a compound of formula (I), or a stereoisomer or tautomer thereof, or a pharmaceutically acceptable salt of any of the foregoing, wherein the compound is a compound of formula (I-G):

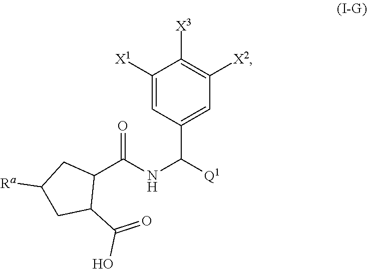

or a stereoisomer or tautomer thereof, or a pharmaceutically acceptable salt of any of the foregoing.

In some embodiments, provided is a compound of formula (I), or (I-G) or a stereoisomer or tautomer thereof, or a pharmaceutically acceptable salt of any of the foregoing, wherein the compound is a compound of formula (I-G1):

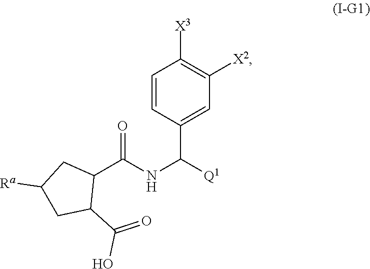

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G2):

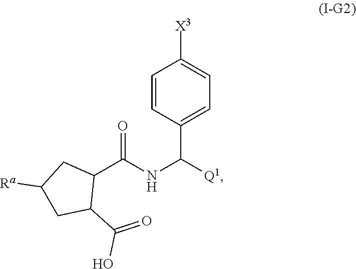

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G3):

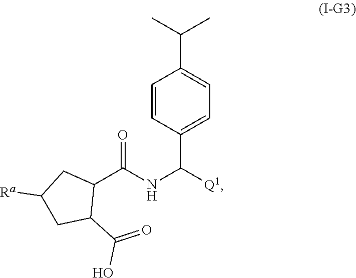

or a stereoisomer or tautomer thereof, or a pharmaceutically acceptable salt of any of the foregoing.

In some embodiments, provided is a compound of formula (I), (I-G), or a stereoisomer or tautomer thereof, or a pharmaceutically acceptable salt of any of the foregoing, wherein the compound is a compound of formula (I-G4):

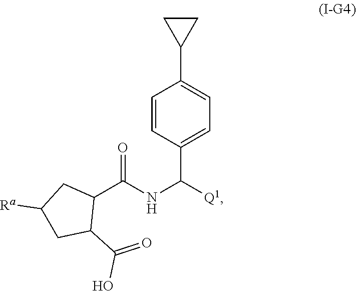

or a stereoisomer or tautomer thereof, or a pharmaceutically acceptable salt of any of the foregoing.

In some embodiments, provided is a compound of formula (I), or a stereoisomer or tautomer thereof, or a pharmaceutically acceptable salt of any of the foregoing, wherein the compound is a compound of formula (I-H):

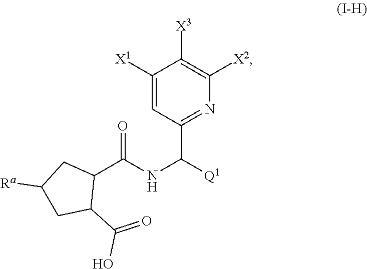

or a stereoisomer or tautomer thereof, or a pharmaceutically acceptable salt of any of the foregoing.

In some embodiments, provided is a compound of formula (I), or (I-H) or a stereoisomer or tautomer thereof, or a pharmaceutically acceptable salt of any of the foregoing, wherein the compound is a compound of formula (I-H1):

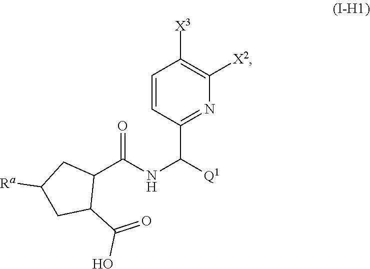

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H2):

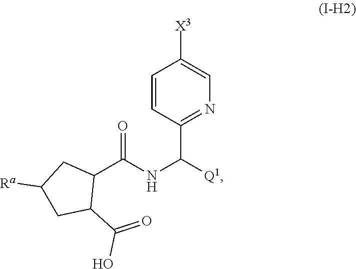

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H3):

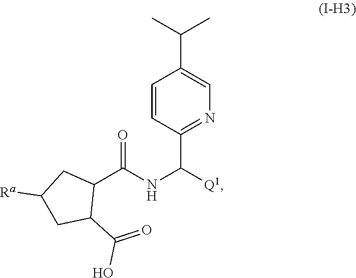

or a stereoisomer or tautomer thereof, or a pharmaceutically acceptable salt of any of the foregoing.

In some embodiments, provided is a compound of formula (I), (I-H), or a stereoisomer or tautomer thereof, or a pharmaceutically acceptable salt of any of the foregoing, wherein the compound is a compound of formula (I-H4):

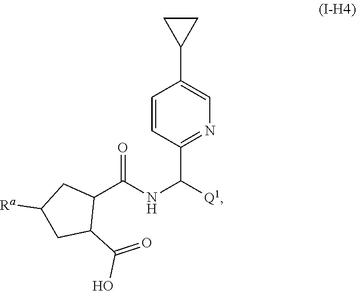

or a stereoisomer or tautomer thereof, or a pharmaceutically acceptable salt of any of the foregoing.

In some embodiments of a compound of formula (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1.

Compound Names included in Table 1 and for all intermediates and compounds were generated using ChemDraw® Professional software version 17.1.1.0 or Collaborative Drug Discovery Inc. (CDD) CDD Vault update #3.

A Knime workflow was created to retrieve structures from an internal ChemAxon Compound Registry, generate the canonical smiles using RDKit Canon SMILES node, remove the stereochemistry using ChemAxon/Infocom MolConverter node, and name the structure using ChemAxon/Infocom Naming node. The following denotes the version of the Knime Analytics Platform and extensions utilized in the workflow:
Knime Analytics Platform 4.2.2
RDKit Knime Integration 4.0.1.v202006261025 (this extension includes the RDKit Canon SMILES node)
ChemAxon/Infocom Marvin Extensions Feature 4.3.0v202100 (this extension includes the MolConverter node)
ChemAxon/Infocom JChem Extensions Feature 4.3.0v202100 (this extension includes the Naming node)

TABLE 1

| Cmpd No. | Structure | Cmpd Name |
|---|---|---|
| 1 | 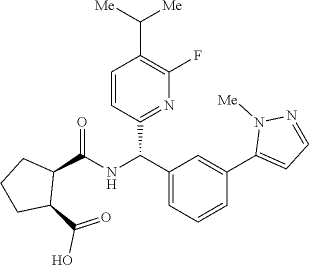 [SMILES: CC(C)c1ccc([C@@H](NC(=O)[C@@H]2CCC[C@@H]2C(=O)O)c2cccc(-c3ccnn3C)c2)nc1F] | (1S,2R)-2-(((S)-(6-fluoro-5- isopropylpyridin-2-yl)(3-(1- methyl-1H-pyrazol-5- yl)phenyl)methyl)carbamoyl)cyclo- pentane-1-carboxylic acid |
| 2 | 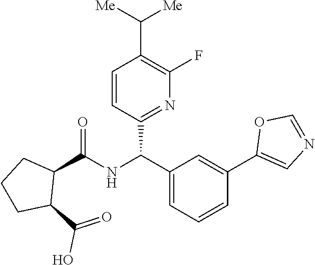 [SMILES: CC(C)c1ccc([C@@H](NC(=O)[C@@H]2CCC[C@@H]2C(=O)O)c2cccc(-c3cnco3)c2)nc1F] | (1S,2R)-2-(((S)-(6-fluoro-5- isopropylpyridin-2-yl)(3-oxazol- 5- yl)phenyl)methyl)carbamoyl)cyclo- pentane-1-carboxylic acid |
| 3 | 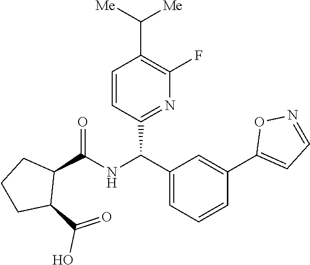 [SMILES: CC(C)c1ccc([C@@H](NC(=O)[C@@H]2CCC[C@@H]2C(=O)O)c2cccc(-c3ccno3)c2)nc1F] | (1S,2R)-2-(((S)-(6-fluoro-5- isopropylpyridin-2-yl)(3- (isoxazol-5- yl)phenyl)methyl)carbamoyl)cyclo- pentane-1-carboxylic acid |
| 4 | 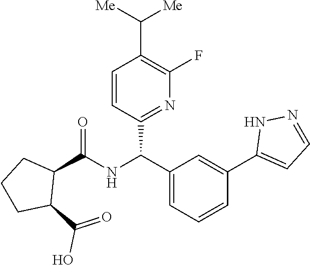 [SMILES: CC(C)c1ccc([C@@H](NC(=O)[C@@H]2CCC[C@@H]2C(=O)O)c2cccc(-c3ccn[nH]3)c2)nc1F] | (1S,2R)-2-(((S)-(3-(1H-pyrazol-5- yl)phenyl)(6-fluoro-5- isopropylpyridin-2- yl)methyl)carbamoyl)cyclopentane- 1-carboxylic acid |
| 5 | 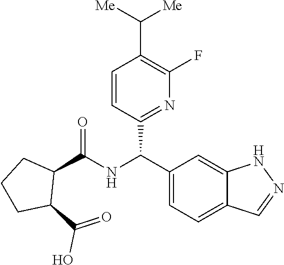 [SMILES: CC(C)c1ccc([C@@H](NC(=O)[C@@H]2CCC[C@@H]2C(=O)O)c2ccc3cn[nH]c3c2)nc1F] | (1S,2R)-2-(((S)-(6-fluoro-5- isopropylpyridin-2-yl)(1H- indazol-6- yl)methyl)carbamoyl)cyclopentane- 1-carboxylic acid |
| 6 | 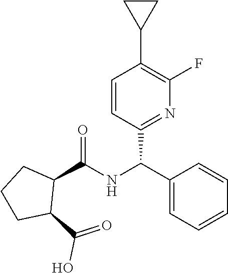 [SMILES: O=C(O)[C@H]1CCC[C@H]1C(=O)N[C@@H](c1ccccc1)c1ccc(C2CC2)c(F)n1] | (1S,2R)-2-(((S)-(5-cyclopropyl-6- fluoropyridin-2- yl)(phenyl)methyl)carbamoyl) cyclopentane-1-carboxylic acid |
| 7 | 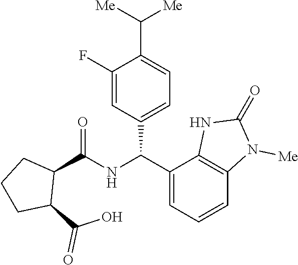 [SMILES: CC(C)c1ccc([C@@H](NC(=O)[C@@H]2CCC[C@@H]2C(=O)O)c2cccc3c2[nH]c(=O)n3C)cc1F] | (1S,2R)-2-(((R)-(3-fluoro-4- isopropylphenyl)(1-methyl-2-oxo- 2,3-dihydro-1H-benzo[b]imidazol- 4- yl)methyl)carbamoyl)cyclopentane- 1-carboxylic acid |
| 8 | 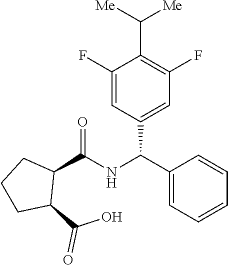 [SMILES: CC(C)c1c(F)cc([C@@H](NC(=O)[C@@H]2CCC[C@@H]2C(=O)O)c2ccccc2)cc1F] | (1S,2R)-2-(((S)-(3,5-difluoro-4- isopropylphenyl)(phenyl)methyl) carbamoyl)cyclopentane-1- carboxylic acid |
| 9 | 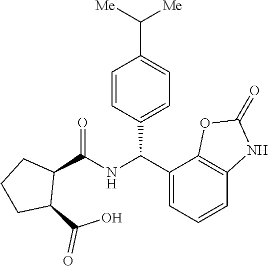 [SMILES: CC(C)c1ccc([C@@H](NC(=O)[C@@H]2CCC[C@@H]2C(=O)O)c2cccc3[nH]c(=O)oc23)cc1] | (1S,2R)-2-(((R)-(4- isopropylphenyl)(2-oxo-2,3- dihydrobenzo[d]oxazol-7- yl)methyl)carbamoyl)cyclopentane- 1-carboxylic acid |
| 10 | 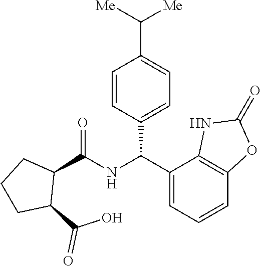 [SMILES: CC(C)c1ccc([C@@H](NC(=O)[C@@H]2CCC[C@@H]2C(=O)O)c2cccc3oc(=O)[nH]c23)cc1] | (1S,2R)-2-(((R)-(4- isopropylphenyl)(2-oxo-2,3- dihydrobenzo[d]oxazol-4- yl)methyl)carbamoyl)cyclopentane- 1-carboxylic acid |
| 11 | 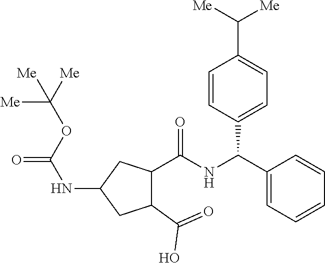 [SMILES: CC(C)c1ccc([C@@H](NC(=O)C2CC(NC(=O)OC(C)(C)C)CC2C(=O)O)c2ccccc2)cc1] | (1S or 1R,2R or 2S,4R or 4S)-4- ((tert-butoxycarbonyl)amino)-2- (((S)-(4- isopropylphenyl)(phenyl)methyl) carbamoyl)cyclopentane-1- carboxylic acid |
| 12 | 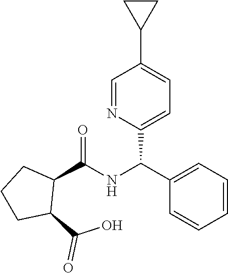 [SMILES: O=C(O)[C@H]1CCC[C@H]1C(=O)N[C@@H](c1ccccc1)c1ccc(C2CC2)cn1] | (1S,2R)-2-(((S)-(5- cyclpropylpyridin-2- yl)(phenyl)methyl)carbamoyl) cyclopentane-1-carboxylic acid |
| 13 | 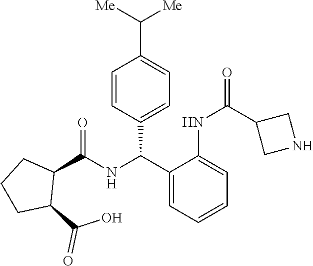 [SMILES: CC(C)c1ccc([C@@H](NC(=O)[C@@H]2CCC[C@@H]2C(=O)O)c2ccccc2NC(=O)C2CNC2)cc1] | (1S,2R)-2-(((R)-(2-(azetidine-3- carboxamido)phenyl)(4- isopropylphenyl)methyl) carbamoyl)cyclopentane-1- carboxylic acid |
| 14 | 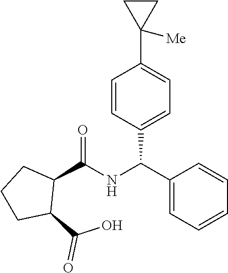 [SMILES: CC1(c2ccc([C@@H](NC(=O)[C@@H]3CCC[C@@H]3C(=O)O)c3ccccc3)cc2)CC1] | (1S,2R)-2-(((S)-(4-(1- methylcyclopropyl)phenyl) (phenyl)methyl)carbamoyl) cyclopentane-1-carboxylic acid |
| 15 | 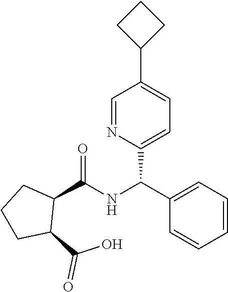 [SMILES: O=C(O)[C@H]1CCC[C@H]1C(=O)N[C@@H](c1ccccc1)c1ccc(C2CCC2)cn1] | (1S,2R)-2-(((S)-(5- cyclobutylpyridin-2- yl)(phenyl)methyl)carbamoyl) cyclopentane-1-carboxylic acid |
| 16 | 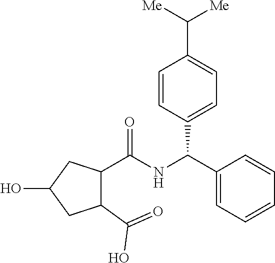 [SMILES: CC(C)c1ccc([C@@H](NC(=O)C2CC(O)CC2C(=O)O)c2ccccc2)cc1] | (1S or 1R,2R or 2S,4R or 4S)-4- hydroxy-2-(((S)-(4- isopropylphenyl)(phenyl)methyl) carbamoyl)cyclopentane-1- carboxylic acid |
| 17 | 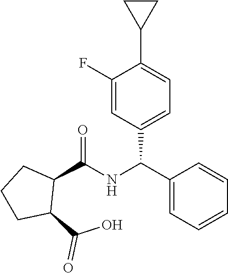 [SMILES: O=C(O)[C@H]1CCC[C@H]1C(=O)N[C@@H](c1ccccc1)c1ccc(C2CC2)c(F)c1] | (1S,2R)-2-(((S)-(4-cyclopropyl-3- fluorophenyl)(phenyl)methyl) carbamoyl)cyclopentane-1- carboxylic acid |
| 18 | 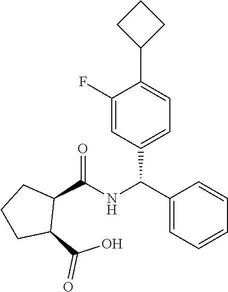 [SMILES: O=C(O)[C@H]1CCC[C@H]1C(=O)N[C@@H](c1ccccc1)c1ccc(C2CCC2)c(F)c1] | (1S,2R)-2-(((S)-(4-cyclobutyl-3- fluorophenyl)(phenyl)methyl) carbamoyl)cyclopentane-1- carboxylic acid |
| 19 | 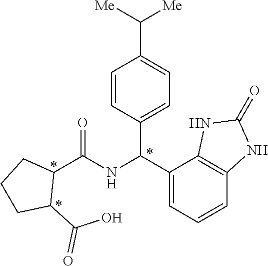 [SMILES: CC(C)c1ccc(C(NC(=O)C2CCCC2C(=O)O)c2cccc3[nH]c(=O)[nH]c23)cc1] | (1S or 1R,2R or 2S)-2-(((R or S)- (4-isopropylphenyl)(2-oxo-2,3- dihydro-1H-benzo[d]imidazol-4- yl)methyl)carbamoyl)cyclopentane- 1-carboxylic acid |
| 20 | 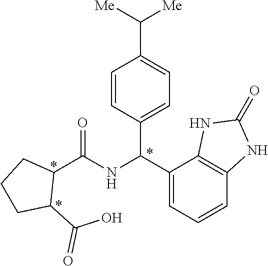 [SMILES: CC(C)c1ccc(C(NC(=O)C2CCCC2C(=O)O)c2cccc3[nH]c(=O)[nH]c23)cc1] | (1R or 1S,2S or 2R)-2-(((S or R)- (4-isopropylphenyl)(2-oxo-2,3- dihydro-1H-benzo[d]imidazol-4- yl)methyl)carbamoyl)cyclopentane- 1-carboxylic acid |
| 21 | 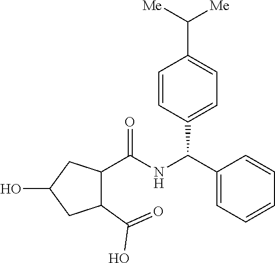 [SMILES: CC(C)c1ccc([C@@H](NC(=O)C2CC(O)CC2C(=O)O)c2ccccc2)cc1] | (1S or 1R,2R or 2S,4S or 4R)-4- hydroxy-2-(((S)-(4- isopropylphenyl)(phenyl)methyl) carbamoyl)cyclopentane-1- carboxylic acid |
| 22 | 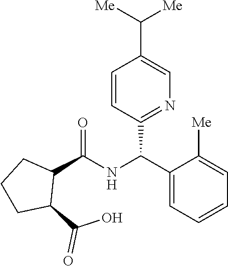 | (1S,2R)-2-(((S)-(5- isopropylpyridin-2-yl)(o- tolyl)methyl)carbamoyl) cyclopentane-1-carboxylic acid |
| 23 | 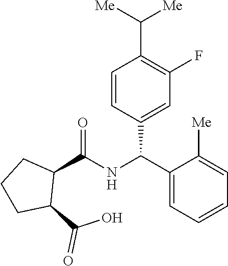 [SMILES: Cc1ccccc1[C@H](NC(=O)[C@@H]1CCC[C@@H]1C(=O)O)c1ccc(C(C)C)c(F)c1] | (1S,2R)-2-(((R)-(3-fluoro-4- isopropylphenyl)(o- tolyl)methyl)carbamoyl) cyclopentane-1-carboxylic acid |
| 24 | 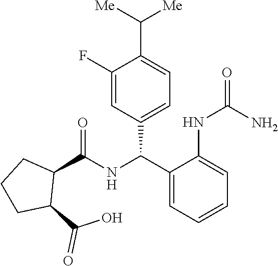 [SMILES: CC(C)c1ccc([C@@H](NC(=O)[C@@H]2CCC[C@@H]2C(=O)O)c2ccccc2NC(N)=O)cc1F] | (1S,2R)-2-(((R)-(3-fluoro-4- isopropylphenyl)(2- ureidophenyl)methyl)carbamoyl) cyclopentane-1-carboxylic acid |
| 25 | 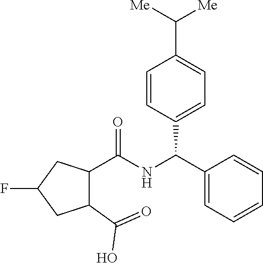 [SMILES: CC(C)c1ccc([C@@H](NC(=O)C2CC(F)CC2C(=O)O)c2ccccc2)cc1] | (1S or 1R,2R or 2S,4R or 4S)-4- fluoro-2-(((S)-(4- isopropylphenyl)(phenyl)methyl) carbamoyl)cyclopentane-1- carboxylic acid |
| 26 | 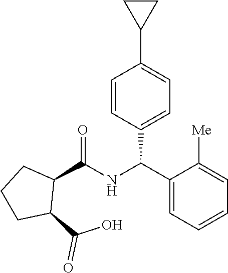 [SMILES: Cc1ccccc1[C@H](NC(=O)[C@@H]1CCC[C@@H]1C(=O)O)c1ccc(C2CC2)cc1] | (1S,2R)-2-(((R)-(4- cyclopropylphenyl)(o- tolyl)methyl)carbamoyl) cyclopentane-1-carboxylic acid |
| 27 | 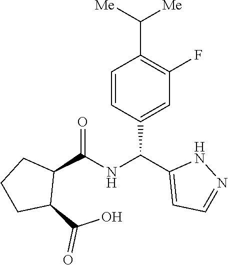 [SMILES: CC(C)c1ccc([C@@H](NC(=O)[C@@H]2CCC[C@@H]2C(=O)O)c2ccn[nH]2)cc1F] | (1S,2R)-2-(((R)-(3-fluoro-4- isopropylphenyl)(1H-pyrazol-5- yl)methyl)carbamoyl)cyclopentane- 1-carboxylic acid |
| 28 | 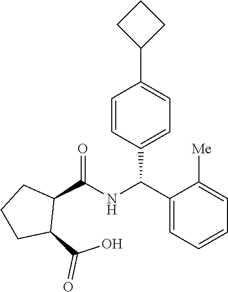 [SMILES: Cc1ccccc1[C@H](NC(=O)[C@@H]1CCC[C@@H]1C(=O)O)c1ccc(C2CCC2)cc1] | (1S,2R)-2-(((R)-(4- cyclobutylphenyl)(o- tolyl)methyl)carbamoyl) cyclopentane-1-carboxylic acid |
| 29 | 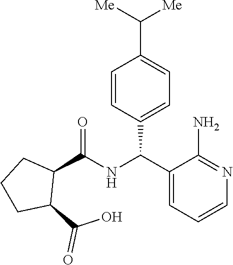 [SMILES: CC(C)c1ccc([C@@H](NC(=O)[C@@H]2CCC[C@@H]2C(=O)O)c2cccnc2N)cc1] | (1S,2R)-2-(((R)-(2-aminopyridin- 3-yl)(4- isopropylphenyl)methyl) carbamoyl)cyclopentane-1- carboxylic acid |
| 30 | 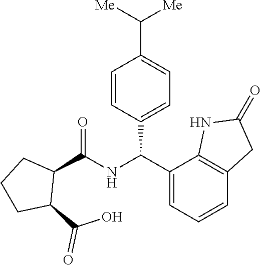 [SMILES: CC(C)c1ccc([C@@H](NC(=O)[C@@H]2CCC[C@@H]2C(=O)O)c2cccc3c2NC(=O)C3)cc1] | (1S,2R)-2-(((R)-(4- isopropylphenyl)(2-oxoindolin-7- yl)methyl)carbamoyl)cyclopentane- 1-carboxylic acid |
| 31 | 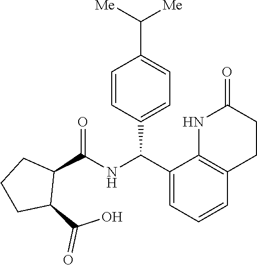 [SMILES: CC(C)c1ccc([C@@H](NC(=O)[C@@H]2CCC[C@@H]2C(=O)O)c2cccc3c2NC(=O)CC3)cc1] | (1S,2R)-2-(((R)-(4- isopropylphenyl)(2-oxo-1,2,3,4- tetrahydroquinolin-8- yl)methyl)carbamoyl)cyclopentane- 1-carboxylic acid |
| 32 | 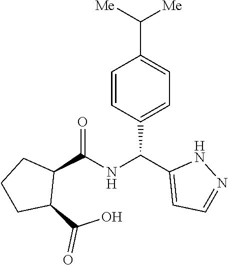 [SMILES: CC(C)c1ccc([C@@H](NC(=O)[C@@H]2CCC[C@@H]2C(=O)O)c2ccn[nH]2)cc1] | (1S,2R)-2-(((R)-(4- isopropylphenyl)(1H-pyrazol-5- yl)methyl)carbamoyl)cyclopentane- 1-carboxylic acid |
| 33 | 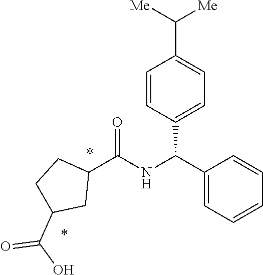 [SMILES: CC(C)c1ccc([C@@H](NC(=O)C2CCC(C(=O)O)C2)c2ccccc2)cc1] | (1S or 1R,3R or 3S)-3-(((S)-(4- isopropylphenyl)(phenyl)methyl) carbamoyl)cyclopentane-1- carboxylic acid |
| 34 | 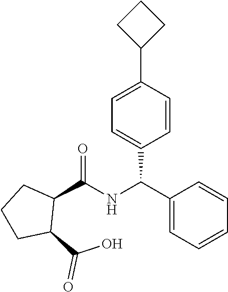 [SMILES: O=C(O)[C@H]1CCC[C@H]1C(=O)N[C@@H](c1ccccc1)c1ccc(C2CCC2)cc1] | (1S,2R)-2-(((S)-(4- cyclobutylphenyl)(phenyl)methyl) carbamoyl)cyclopentane-1- carboxylic acid |
| 35 | 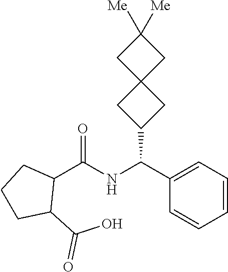 [SMILES: CC1(C)CC2(CC([C@@H](NC(=O)C3CCCC3C(=O)O)c3ccccc3)C2)C1] | (1S or 1R,2R or 2S)-2-(((R)-(6,6- dimethylspiro[3.3]heptan-2- yl)(phenyl)methyl)carbamoyl) cyclopentane-1-carboxylic acid |
| 36 | 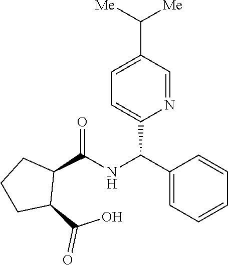 [SMILES: CC(C)c1ccc([C@@H](NC(=O)[C@@H]2CCC[C@@H]2C(=O)O)c2ccccc2)nc1] | (1S,2R)-2-(((S)-(5- isopropylpyridin-2- yl)(phenyl)methyl)carbamoyl) cyclopentane-1-carboxylic acid |
| 37 | 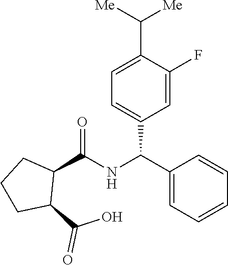 [SMILES: CC(C)c1ccc([C@@H](NC(=O)[C@@H]2CCC[C@@H]2C(=O)O)c2ccccc2)cc1F] | (1S,2R)-2-(((S)-(3-fluoro-4- isopropylphenyl)(phenyl)methyl) carbamoyl)cyclopentane-1- carboxylic acid |
| 38 | 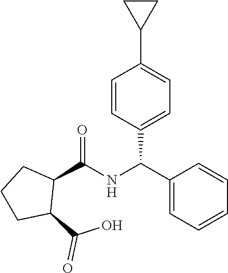 [SMILES: O=C(O)[C@H]1CCC[C@H]1C(=O)N[C@@H](c1ccccc1)c1ccc(C2CC2)cc1] | (1S,2R)-2-(((S)-(4- cyclopropylphenyl)(phenyl)methyl) carbamoyl)cyclopentane-1- carboxylic acid |
| 39 | 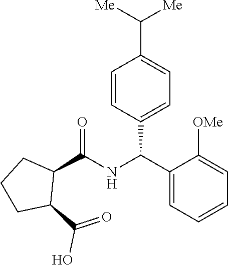 [SMILES: COc1ccccc1[C@H](NC(=O)[C@@H]1CCC[C@@H]1C(=O)O)c1ccc(C(C)C)cc1] | (1S,2R)-2-(((R)-(4- isopropylphenyl)(2- methoxyphenyl)methyl)carbamoyl) cyclopentane-1-carboxylic acid |
| 40 | 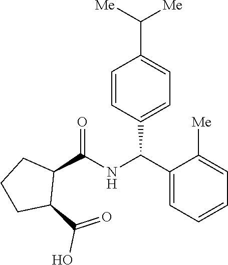 [SMILES: Cc1ccccc1[C@H](NC(=O)[C@@H]1CCC[C@@H]1C(=O)O)c1ccc(C(C)C)cc1] | (1S,2R)-2-(((R)-(4- isopropylphenyl)(o- tolyl)methyl)carbamoyl) cyclopentane-1-carboxylic acid |
| 41 | 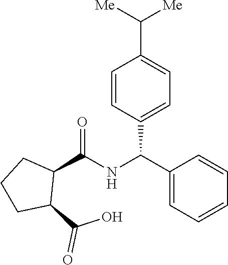 [SMILES: CC(C)c1ccc([C@@H](NC(=O)[C@@H]2CCC[C@@H]2C(=O)O)c2ccccc2)cc1] | (1S,2R)-2-(((S)-(4- isopropylphenyl)(phenyl)methyl) carbamoyl)cyclopentane-1- carboxylic acid |
| 42 | 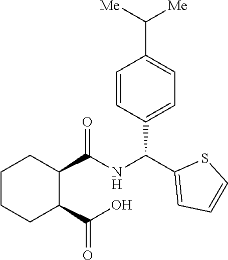 [SMILES: CC(C)c1ccc([C@@H](NC(=O)[C@@H]2CCCC[C@@H]2C(=O)O)c2cccs2)cc1] | (1S,2R)-2-(((R)-(4- isopropylphenyl)(thiophen-2- yl)methyl)carbamoyl)cyclohexane- 1-carboxylic acid |
| 43 | 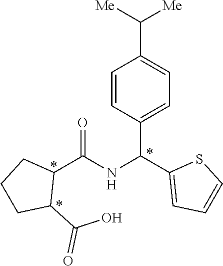 [SMILES: CC(C)c1ccc(C(NC(=O)C2CCCC2C(=O)O)c2cccs2)cc1] | (1R or 1S,2S or 2R)-2-(((S or R)- (4-isopropylphenyl)(thiophen-2- yl)methyl)carbamoyl)cyclopentane- 1-carboxylic acid |
| 44 | 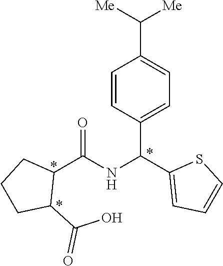 [SMILES: CC(C)c1ccc(C(NC(=O)C2CCCC2C(=O)O)c2cccs2)cc1] | (1R or 1S,2S or 2R)-2-(((R or S)- (4-isopropylphenyl)(thiophen-2- yl)methyl)carbamoyl)cyclopentane- 1-carboxylic acid |
| 45 | 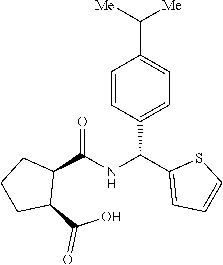 [SMILES: CC(C)c1ccc([C@@H](NC(=O)[C@@H]2CCC[C@@H]2C(=O)O)c2cccs2)cc1] | (1S,2R)-2-(((R)-(4- isopropylphenyl)(thiophen-2- yl)methyl)carbamoyl)cyclopentane- 1-carboxylic acid |
| 46 | 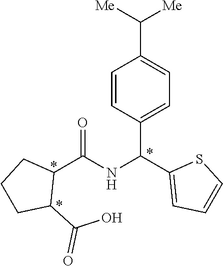 [SMILES: CC(C)c1ccc(C(NC(=O)C2CCCC2C(=O)O)c2cccs2)cc1] | (1S or 1R,2R or 2S)-2-(((S or R)- (4-isopropylphenyl)(thiophen-2- yl)methyl)carbamoyl)cyclopentane- 1-carboxylic acid |

In some embodiments, provided herein is a compound of formula (I), or any variation or embodiment thereof, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from the group consisting of:
2-({[4-(propan-2-yl)phenyl](thiophen-2-yl)methyl}carbamoyl)cyclopentane-1-carboxylic acid;
2-({[4-(propan-2-yl)phenyl](thiophen-2-yl)methyl}carbamoyl)cyclohexane-1-carboxylic acid;
2-({phenyl[4-(propan-2-yl)phenyl]methyl}carbamoyl)cyclopentane-1-carboxylic acid;
2-{[(2-methylphenyl) [4-(propan-2-yl)phenyl]methyl]carbamoyl}cyclopentane-1-carboxylic acid;
2-{[(2-methoxyphenyl) [4-(propan-2-yl)phenyl]methyl]carbamoyl}cyclopentane-1-carboxylic acid;
2-{[(4-cyclopropylphenyl)(phenyl)methyl]carbamoyl}cyclopentane-1-carboxylic acid;
2-({[3-fluoro-4-(propan-2-yl)phenyl](phenyl)methyl}carbamoyl)cyclopentane-1-carboxylic acid;
2-({phenyl[5-(propan-2-yl) pyridin-2-yl]methyl}carbamoyl)cyclopentane-1-carboxylic acid;
2-[({6,6-dimethylspiro[3.3]heptan-2-yl}(phenyl)methyl) carbamoyl]cyclopentane-1-carboxylic acid;
3-({phenyl[4-(propan-2-yl)phenyl]methyl}carbamoyl)cyclopentane-1-carboxylic acid;
2-{[(4-cyclobutylphenyl)(phenyl)methyl]carbamoyl}cyclopentane-1-carboxylic acid;
2-({[4-(propan-2-yl)phenyl](1H-pyrazol-5-yl)methyl}carbamoyl)cyclopentane-1-carboxylic acid;
2-{[(2-oxo-1,2,3,4-tetrahydroquinolin-8-yl) [4-(propan-2-yl)phenyl]methyl]carbamoyl}cyclopentane-1-carboxylic acid;
2-{[(2-oxo-2,3-dihydro-1H-indol-7-yl) [4-(propan-2-yl)phenyl]methyl]carbamoyl}cyclopentane-1-carboxylic acid;
2-{[(2-aminopyridin-3-yl) [4-(propan-2-yl)phenyl]methyl]carbamoyl}cyclopentane-1-carboxylic acid;
2-{[(4-cyclobutylphenyl)(2-methylphenyl)methyl]carbamoyl}cyclopentane-1-carboxylic acid;
2-({[3-fluoro-4-(propan-2-yl)phenyl](1H-pyrazol-5-yl)methyl}carbamoyl)cyclopentane-1-carboxylic acid;
2-{[(4-cyclopropylphenyl)(2-methylphenyl)methyl]carbamoyl}cyclopentane-1-carboxylic acid;
4-fluoro-2-({phenyl[4-(propan-2-yl)phenyl]methyl}carbamoyl)cyclopentane-1-carboxylic acid;
2-({[2-(carbamoylamino)phenyl][3-fluoro-4-(propan-2-yl)phenyl]methyl}carbamoyl)cyclopentane-1-carboxylic acid;
2-({[3-fluoro-4-(propan-2-yl)phenyl](2-methylphenyl)methyl}carbamoyl)cyclopentane-1-carboxylic acid;
2-{[(2-methylphenyl) [5-(propan-2-yl) pyridin-2-yl]methyl]carbamoyl}cyclopentane-1-carboxylic acid;
4-hydroxy-2-({phenyl[4-(propan-2-yl)phenyl]methyl}carbamoyl)cyclopentane-1-carboxylic acid;
2-{[(2-oxo-2,3-dihydro-1H-1,3-benzodiazol-4-yl) [4-(propan-2-yl)phenyl]methyl]carbamoyl}cyclopentane-1-carboxylic acid;
2-{[(4-cyclobutyl-3-fluorophenyl)(phenyl)methyl]carbamoyl}cyclopentane-1-carboxylic acid;
2-{[(4-cyclopropyl-3-fluorophenyl)(phenyl)methyl]carbamoyl}cyclopentane-1-carboxylic acid;
4-hydroxy-2-({phenyl[4-(propan-2-yl)phenyl]methyl}carbamoyl)cyclopentane-1-carboxylic acid;
2-{[(5-cyclobutylpyridin-2-yl)(phenyl)methyl]carbamoyl}cyclopentane-1-carboxylic acid;
2-({[4-(1-methylcyclopropyl)phenyl](phenyl)methyl}carbamoyl)cyclopentane-1-carboxylic acid;
2-({[2-(azetidine-3-amido)phenyl][4-(propan-2-yl)phenyl]methyl}carbamoyl)cyclopentane-1-carboxylic acid;
2-{[(5-cyclopropylpyridin-2-yl)(phenyl)methyl]carbamoyl}cyclopentane-1-carboxylic acid;
4-{[(tert-butoxy) carbonyl]amino}-2-({phenyl[4-(propan-2-yl)phenyl]methyl}carbamoyl)cyclopentane-1-carboxylic acid;
2-{[(2-oxo-2,3-dihydro-1,3-benzoxazol-4-yl) [4-(propan-2-yl)phenyl]methyl]carbamoyl}cyclopentane-1-carboxylic acid;
2-{[(2-oxo-2,3-dihydro-1,3-benzoxazol-7-yl) [4-(propan-2-yl)phenyl]methyl]carbamoyl}cyclopentane-1-carboxylic acid;
2-({[3,5-difluoro-4-(propan-2-yl)phenyl](phenyl)methyl}carbamoyl)cyclopentane-1-carboxylic acid;
2-({[3-fluoro-4-(propan-2-yl)phenyl](1-methyl-2-oxo-2,3-dihydro-1H-1,3-benzodiazol-4-yl)methyl}carbamoyl)cyclopentane-1-carboxylic acid;
   2-{[(5-cyclopropyl-6-fluoropyridin-2-yl)(phenyl)methyl]carbamoyl}cyclopentane-1-carboxylic acid;
2-({[6-fluoro-5-(propan-2-yl) pyridin-2-yl](1H-indazol-6-yl)methyl}carbamoyl)cyclopentane-1-carboxylic acid;
2-({[6-fluoro-5-(propan-2-yl) pyridin-2-yl][3-(1H-pyrazol-5-yl)phenyl]methyl}carbamoyl)cyclopentane-1-carboxylic acid;
2-({[6-fluoro-5-(propan-2-yl) pyridin-2-yl][3-(1,2-oxazol-5-yl)phenyl]methyl}carbamoyl)cyclopentane-1-carboxylic acid;
2-({[6-fluoro-5-(propan-2-yl) pyridin-2-yl][3-(1,3-oxazol-5-yl)phenyl]methyl}carbamoyl)cyclopentane-1-carboxylic acid; and
2-({[6-fluoro-5-(propan-2-yl) pyridin-2-yl][3-(1-methyl-1H-pyrazol-5-yl)phenyl]methyl}carbamoyl)cyclopentane-1-carboxylic acid;
   or a tautomer thereof, or a pharmaceutically acceptable salt of any of the foregoing.

In some embodiments, provided herein is a compound of formula (I), or any variation or embodiment thereof, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from the group consisting of:
(1S,2R)-2-(((S)-(6-fluoro-5-isopropylpyridin-2-yl)(3-(1-methyl-1H-pyrazol-5-yl)phenyl)methyl) carbamoyl)cyclopentane-1-carboxylic acid;
(1S,2R)-2-(((S)-(6-fluoro-5-isopropylpyridin-2-yl)(3-(oxazol-5-yl)phenyl)methyl) carbamoyl)cyclopentane-1-carboxylic acid;
(1S,2R)-2-(((S)-(6-fluoro-5-isopropylpyridin-2-yl)(3-(isoxazol-5-yl)phenyl)methyl) carbamoyl)cyclopentane-1-carboxylic acid;
(1S,2R)-2-(((S)-(3-(1H-pyrazol-5-yl)phenyl)(6-fluoro-5-isopropylpyridin-2-yl)methyl) carbamoyl)cyclopentane-1-carboxylic acid;
   (1S,2R)-2-(((S)-(6-fluoro-5-isopropylpyridin-2-yl)(1H-indazol-6-yl)methyl) carbamoyl)cyclopentane-1-carboxylic acid;
(1S,2R)-2-(((S)-(5-cyclopropyl-6-fluoropyridin-2-yl)(phenyl)methyl) carbamoyl)cyclopentane-1-carboxylic acid;
   (1S,2R)-2-(((R)-(3-fluoro-4-isopropylphenyl)(1-methyl-2-oxo-2,3-dihydro-1H-benzo[d]imidazol-4-yl)methyl) carbamoyl)cyclopentane-1-carboxylic acid;
   (1S,2R)-2-(((S)-(3,5-difluoro-4-isopropylphenyl)(phenyl)methyl) carbamoyl)cyclopentane-1-carboxylic acid;
   (1S,2R)-2-(((R)-(4-isopropylphenyl)(2-oxo-2,3-dihydrobenzo[d]oxazol-7-yl)methyl) carbamoyl)cyclopentane-1-carboxylic acid;
(1S,2R)-2-(((R)-(4-isopropylphenyl)(2-oxo-2,3-dihydrobenzo[d]oxazol-4-yl)methyl) carbamoyl)cyclopentane-1-carboxylic acid;
(1S or 1R,2R or 2S,4R or 4S)-4-((tert-butoxycarbonyl)amino)-2-(((S)-(4-isopropylphenyl)(phenyl)methyl) carbamoyl)cyclopentane-1-carboxylic acid;
(1S,2R)-2-(((S)-(5-cyclopropylpyridin-2-yl)(phenyl)methyl) carbamoyl)cyclopentane-1-carboxylic acid;
   (1S,2R)-2-(((R)-(2-(azetidine-3-carboxamido)phenyl)(4-isopropylphenyl)methyl) carbamoyl)cyclopentane-1-carboxylic acid;
(1S,2R)-2-(((S)-(4-(1-methylcyclopropyl)phenyl)(phenyl)methyl) carbamoyl)cyclopentane-1-carboxylic acid;
   (1S,2R)-2-(((S)-(5-cyclobutylpyridin-2-yl)(phenyl)methyl) carbamoyl)cyclopentane-1-carboxylic acid;
   (1S or 1R,2R or 2S,4R or 4S)-4-hydroxy-2-(((S)-(4-isopropylphenyl)(phenyl)methyl) carbamoyl)cyclopentane-1-carboxylic acid;
(1S,2R)-2-(((S)-(4-cyclopropyl-3-fluorophenyl)(phenyl)methyl) carbamoyl)cyclopentane-1-carboxylic acid;
   (1S,2R)-2-(((S)-(4-cyclobutyl-3-fluorophenyl)(phenyl)methyl) carbamoyl)cyclopentane-1-carboxylic acid;
   (1S,2R)-2-(((R)-(4-isopropylphenyl)(2-oxo-2,3-dihydro-1H-benzo[d]imidazol-4-yl)methyl) carbamoyl)cyclopentane-1-carboxylic acid;
(1S,2R)-2-(((R)-(4-isopropylphenyl)(2-oxo-2,3-dihydro-1H-benzo[d]imidazol-4-yl)methyl) carbamoyl)cyclopentane-1-carboxylic acid;
   (1S or 1R,2R or 2S,4S or 4R)-4-hydroxy-2-(((S)-(4-isopropylphenyl)(phenyl)methyl) carbamoyl)cyclopentane-1-carboxylic acid;
(1S,2R)-2-(((S)-(5-isopropylpyridin-2-yl)(o-tolyl)methyl) carbamoyl)cyclopentane-1-carboxylic acid;
(1S,2R)-2-(((R)-(3-fluoro-4-isopropylphenyl)(o-tolyl)methyl) carbamoyl)cyclopentane-1-carboxylic acid;
(1S,2R)-2-(((R)-(3-fluoro-4-isopropylphenyl)(2-ureidophenyl)methyl) carbamoyl)cyclopentane-1-carboxylic acid; (1S or 1R,2R or 2S,4R or 4S)-4-fluoro-2-(((S)-(4-isopropylphenyl)(phenyl)methyl) carbamoyl)cyclopentane-1-carboxylic acid;
(1S,2R)-2-(((R)-(4-cyclopropylphenyl)(o-tolyl)methyl) carbamoyl)cyclopentane-1-carboxylic acid;
(1S,2R)-2-(((R)-(3-fluoro-4-isopropylphenyl)(1H-pyrazol-5-yl)methyl) carbamoyl)cyclopentane-1-carboxylic acid;
(1S,2R)-2-(((R)-(4-cyclobutylphenyl)(o-tolyl)methyl) carbamoyl)cyclopentane-1-carboxylic acid;
(1S,2R)-2-(((R)-(2-aminopyridin-3-yl)(4-isopropylphenyl)methyl) carbamoyl)cyclopentane-1-carboxylic acid;
(1S,2R)-2-(((R)-(4-isopropylphenyl)(2-oxoindolin-7-yl)methyl) carbamoyl)cyclopentane-1-carboxylic acid;
(1S,2R)-2-(((R)-(4-isopropylphenyl)(2-oxo-1,2,3,4-tetrahydroquinolin-8-yl)methyl) carbamoyl)cyclopentane-1-carboxylic acid;
(1S,2R)-2-(((R)-(4-isopropylphenyl)(1H-pyrazol-5-yl)methyl) carbamoyl)cyclopentane-1-carboxylic acid;
(1S or 1R,3R or 3S)-3-(((S)-(4-isopropylphenyl)(phenyl)methyl) carbamoyl)cyclopentane-1-carboxylic acid;
(1S,2R)-2-(((S)-(4-cyclobutylphenyl)(phenyl)methyl) carbamoyl)cyclopentane-1-carboxylic acid;
(1S or 1R,2R or 2S)-2-(((R)-(6,6-dimethylspiro[3.3]heptan-2-yl)(phenyl)methyl) carbamoyl)cyclopentane-1-carboxylic acid;
(1S,2R)-2-(((S)-(5-isopropylpyridin-2-yl)(phenyl)methyl) carbamoyl)cyclopentane-1-carboxylic acid;
(1S,2R)-2-(((S)-(3-fluoro-4-isopropylphenyl)(phenyl)methyl) carbamoyl)cyclopentane-1-carboxylic acid;
(1S,2R)-2-(((S)-(4-cyclopropylphenyl)(phenyl)methyl) carbamoyl)cyclopentane-1-carboxylic acid;
(1S,2R)-2-(((R)-(4-isopropylphenyl)(2-methoxyphenyl)methyl) carbamoyl)cyclopentane-1-carboxylic acid;
(1S,2R)-2-(((R)-(4-isopropylphenyl)(o-tolyl)methyl) carbamoyl)cyclopentane-1-carboxylic acid;
(1S,2R)-2-(((S)-(4-isopropylphenyl)(phenyl)methyl) carbamoyl)cyclopentane-1-carboxylic acid;
(1S,2R)-2-(((R)-(4-isopropylphenyl)(thiophen-2-yl)methyl) carbamoyl)cyclohexane-1-carboxylic acid;
(1R or 1S,2S or 2R)-2-(((S or R)-(4-isopropylphenyl)(thiophen-2-yl)methyl) carbamoyl)cyclopentane-1-carboxylic acid;
(1R or 1S,2S or 2R)-2-(((R or S)-(4-isopropylphenyl)(thiophen-2-yl)methyl) carbamoyl)cyclopentane-1-carboxylic acid;
(1S,2R)-2-(((R)-(4-isopropylphenyl)(thiophen-2-yl)methyl) carbamoyl)cyclopentane-1-carboxylic acid; and
(1S or 1R,2R or 2S)-2-(((S or R)-(4-isopropylphenyl)(thiophen-2-yl)methyl) carbamoyl)cyclopentane-1-carboxylic acid;
   or a stereoisomer or tautomer thereof, or a pharmaceutically acceptable salt of any of the foregoing.

### Methods of Treatment

Provided herein is a method of modulating GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the compound of formula (I), or any variation or embodiment thereof, or a stereoisomer or tautomer thereof, is selective for GYS1 over GYS2. In some embodiments, the compound of formula (I), or any variation or embodiment thereof, or a stereoisomer or tautomer thereof, is greater than 500 or 1,000 or 1,500 or 1,700-fold selective for GYS1 over GYS2

Provided herein is a method of inhibiting GYS1 in a cell, comprising exposing the cell to (i) a composition comprising an effective amount of a GYS1 inhibitor, or (ii) a pharmaceutical composition, comprising an effective amount of a GYS1 inhibitor, and one or more pharmaceutically acceptable excipients. In some embodiments, the GYS1 inhibitor is a small molecule. In some embodiments, the GYS1 inhibitor is selective for GYS1 over GYS2. In some embodiments, the GYS1 inhibitor is greater than 500 or 1,000 or 1,500 or 1,700-fold selective for GYS1 over GYS2.

Provided herein is a method of inhibiting GYS1 in a cell, comprising exposing the cell to (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

In some embodiments, the compound of formula (I), or any variation or embodiment thereof, or a stereoisomer or tautomer thereof, or a pharmaceutically acceptable salt of any of the foregoing, is selective for GYS1 over GYS2. In some embodiments, the compound of formula (I), or any variation or embodiment thereof, or a stereoisomer or tautomer thereof, or a pharmaceutically acceptable salt of any of the foregoing, is greater than 500 or 1,000 or 1,500 or 1,700-fold selective for GYS1 over GYS2. In some embodiments, the individual has a GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML). In some embodiments, the GYS1-mediated disease, disorder, or condition is Pompe disease. In some embodiments, the GYS1-mediated disease, disorder, or condition is late-onset Pompe disease (LOPD).

Provided herein is a method of reducing tissue glycogen stores in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients.

Provided herein is a method of inhibiting glycogen synthesis in an individual in need thereof, comprising administering to the individual an effective amount of (i) compound of formula (I) or any variation or embodiment thereof, or a stereoisomer or tautomer thereof, or a pharmaceutically acceptable salt of any of the foregoing, or (ii) a pharmaceutical composition, comprising compound of formula (I) or any variation or embodiment thereof, or a stereoisomer or tautomer thereof, or a pharmaceutically acceptable salt of any of the foregoing, and one or more pharmaceutically acceptable excipients.

Provided herein is a method of treating a GYS1-mediated disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the GYS1-mediated disease, disorder, or condition is selected from the group consisting of Pompe disease, Cori disease (GSD III), adult polyglucosan body disease (APBD), and Lafora disease. In some embodiments, the GYS1-mediated disease, disorder, or condition is cancer. In some embodiments, the GYS1-mediated disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

Provided herein is a method of treating a glycogen storage disease, disorder, or condition in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating a glycogen storage disease, disorder, or condition in an individual in need thereof, comprising administering to the individual (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the level of glycogen in the individual is reduced upon treatment. In some embodiments, the level of glycogen in muscle is reduced. In some embodiments, the level of glycogen is skeletal muscle is reduced. In some embodiments, the level of glycogen is reduced at least 10%, at least 20%, at least 30% or at least 50% upon administration of the compound. In some embodiments, the compounds provided herein are effective for treating a lysosomal disorder. In some embodiments, the glycogen storage disease, disorder, or condition is selected from the group consisting of Pompe disease, Cori disease (GSD III), adult polyglucosan body disease (APBD), and Lafora disease.

Provided herein is a method of treating Pompe disease in an individual in need thereof, comprising administering to the individual (i) a composition comprising an effective amount of a compound of formula (I), or any variation or embodiment thereof, or a stereoisomer or tautomer thereof, or a pharmaceutically acceptable salt of any of the foregoing, or (ii) a pharmaceutical composition, comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

Provided herein is a method of treating Pompe disease in an individual in need thereof, comprising administering to the individual (i) a compound of formula (I), or any variation or embodiment thereof, or a stereoisomer or tautomer thereof, or a pharmaceutically acceptable salt of any of the foregoing, or (ii) a pharmaceutical composition, comprising a compound of formula (I), or any variation or embodiment thereof, or a stereoisomer or tautomer thereof, or a pharmaceutically acceptable salt of any of the foregoing, and one or more pharmaceutically acceptable excipients. In some embodiments, the individual has infant onset Pompe disease. In some embodiments, the individual has non-classic infant-onset Pompe disease. In some embodiments, the individual has late-onset Pompe disease. In some embodiments, the individual has a deficiency in acid alfa glucosidase (GAA). In some embodiments, the individual has reduced expression of GAA.

In some embodiments, the compounds provided herein reduce and/or eliminate one or more symptoms associated with Pompe disease. In some embodiments, the compounds reduce and/or eliminate weak muscles, poor muscle tone, enlarged liver, failure to grow and gain weight, trouble breathing, feeding problems, infections in the respiratory system, problems with hearing, motor skill delay, heart enlargement, tiredness, lung infection, frequent falling, or irregular heartbeat. In some embodiments, the compounds herein delay progression of Pompe disease.

In some embodiments, the compounds provided herein increase the lifespan of the individual. In some embodiments, the lifespan is increased at least 5, at least 10, or at least 20 years upon treatment.

In some embodiments, the compounds provided herein prevent, reduce, or delay muscle weakness. In some embodiments, muscle weakness is determined by manual muscle testing, sit to stand test, heel-raise test, hand-held dynamometry, or hand grip dynamometry. In some embodiments, strength is graded according to the following scale: 0: No visible muscle contraction; 1: Visible muscle contraction with no or trace movement: 2: Limb movement, but not against gravity: 3: Movement against gravity but not resistance: 4: Movement against at least some resistance supplied by the examiner: 5: Full strength.

Also provided herein is a method of inhibiting a GYS1 enzyme in an individual comprising administering an effective amount of a compound of formula (I) or a pharmaceutically acceptable salt thereof to the individual. In some embodiments the GYS1 enzyme is human GYS1 (hGYS1). In some embodiments, the compounds provided herein are inhibit GYS1 at a concentration of less than 10 μM, less than 1 μM, less than 0.5 μM, or less than 0.1 μM. In some embodiments, the compounds provided herein inhibit GYS1 at a concentration of 1-10 μM, 0.01 to 1 μM, or 0.01 to 10 μM.

In some embodiments, the compounds have an IC₅₀ of less than 10 nM, less than 10 μM, less than 1 μM, less than 0.5 μM, or less than 0.1 μM. In some embodiments, the compounds provided herein have an IC₅₀ of 1 to 10 nM, 1 to 10 μM, 0.01 to 1 μM, 0.01 to 10 μM, or 0.001 to 0.01 μM.

In some embodiments, glycogen synthesis is inhibited upon administration of a compound provided herein. In some embodiments, glycogen synthesis is reduced at least 10%, at least 20%, at least 40% or at least 50% upon administration.

In some embodiments, the individual receiving treatment is a juvenile human or an infant. In some embodiments, the individual is less than 10 years old, less than 9 years old, less than 8 years old, less than 7 years old, less than 6 years old, less than 5 years old, less than 4 years old, less than 3 years old, less than 2 years old, or less than one year old.

In some embodiments, the methods further comprise enzyme replacement therapy (ERT). Exemplary ERTs include alglucosidase alfa (human recombinant alpha-glucosidase (human GAA)) and those described in Byrne B J et al (2011). Pompe disease: design, methodology, and early findings from the Pompe Registry. Mol Genet Metab 103: 1-11 (herein incorporated by reference in its entirety). In some embodiments, the ERT is selected from the group consisting of Myozyme and Lumizyme. In some embodiments, the ERT is Myozyme. In some embodiments, the ERT is Lumizyme. In some embodiments, the individual has an advanced glycogen storage disease. In some embodiments, the individual has late onset Pompe Disease. Thus, provided herein is a method of treating a GYS1-mediated disease, disorder, or condition in an individual in need thereof, comprising subjecting the individual to (a) glycogen substrate reduction therapy, such as administering to the individual an effective amount of (i) compound of formula (I), or any variation or embodiment thereof, or a stereoisomer or tautomer thereof, or a pharmaceutically acceptable salt of any of the foregoing, or (ii) a pharmaceutical composition comprising compound of formula (I), or any variation or embodiment thereof, or a stereoisomer or tautomer thereof, or a pharmaceutically acceptable salt of any of the foregoing, and one or more pharmaceutically acceptable excipients, and (b) enzyme replacement therapy. In some embodiments, the GYS1-mediated disease, disorder, or condition is Pompe disease, such as late-onset Pompe disease. In some embodiments, the compound of formula (I) is selective for GYS1 over GYS2. In some embodiments, the compound of formula (I) is greater than 500 or 1,000 or 1,500 or 1,700-fold selective for GYS1 over GYS2.

In some embodiments, the individual has a mutation in the GAA gene. In some embodiments, the mutation reduces the level of GAA protein. In some embodiments, the mutation is a loss-of-function mutation. In some embodiments, the mutation is a missense mutation. In some embodiments, the mutation is a deletion. In some embodiments, the mutation is a recessive mutation. In some embodiments, the mutation is a splicing variant.

In some embodiments of the foregoing, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a composition comprising an effective amount of a compound of formula (I), or any variation or embodiment thereof, or a stereoisomer or tautomer thereof, or a pharmaceutically acceptable salt of any of the foregoing, and (ii) instructions for use in treating an GYS1-mediated disease, disorder, or condition in an individual in need thereof. Also provided herein are kits, comprising (i) a pharmaceutical composition comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients; and (ii) instructions for use in treating an GYS1-mediated disease, disorder, or condition in an individual in need thereof

Provided herein are kits, comprising (i) a compound of formula (I), or any variation or embodiment thereof, or a stereoisomer or tautomer thereof, or a pharmaceutically acceptable salt of any of the foregoing, and (ii) instructions for use in treating an GYS1-mediated disease, disorder, or condition in an individual in need thereof. Also provided herein are kits, comprising (i) a pharmaceutical composition comprising an effective amount of a compound of formula (I), or any variation or embodiment thereof, or a stereoisomer or tautomer thereof, or a pharmaceutically acceptable salt of any of the foregoing, and one or more pharmaceutically acceptable excipients; and (ii) instructions for use in treating an GYS1-mediated disease, disorder, or condition in an individual in need thereof

Articles of manufacture are also provided, wherein the article of manufacture comprises a compound of formula (I), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (I), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Preparing

The present disclosure further provides processes for preparing the compounds of present invention. In some aspects, provided herein are processes of preparing a compound of (I), (I-A), (I-A1), (I-A2), (I-B), (I-B1), (I-B2), (I-B3), (I-B4), (I-B5), (I-C), (I-D), (I-E), (I-E1), (I-E2), (I-F), (I-F1), (I-G), (I-G1), (I-G2), (I-G3), (I-G4), (I-H), (I-H1), (I-H2), (I-H3), (I-H4), or (I-H5), or a stereoisomer or tautomer thereof, or a pharmaceutically acceptable salt of any of the foregoing.

In some embodiments, a process for preparing a compound of formula (I), or a stereoisomer or tautomer thereof, or a pharmaceutically acceptable salt of any of the foregoing, comprises:
(a) reacting a compound of formula (I-1):

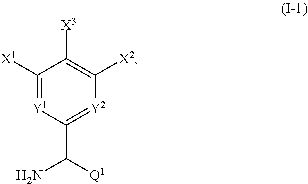

or a salt thereof, wherein
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl; and
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each RD is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1,
   provided that, when X³ is H, then Q¹ is C₆₋₁₀cycloalkyl, wherein the C₆₋₁₀cycloalkyl of Q¹ is optionally substituted with one or more C₁₋₆alkyl;
with a compound of formula (I-2):

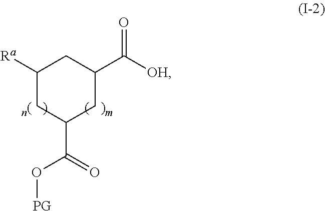

m is 0, or 1, and n is 0, 1, or 2, wherein m+n is an integer from 1 to 2;
R^{a} is H, halo, —OH, or —NH—C(O)—C₁₋₆alkoxy;
and PG is a protecting group;
in the presence of a coupling reagent, to provide a compound of formula (1-3):

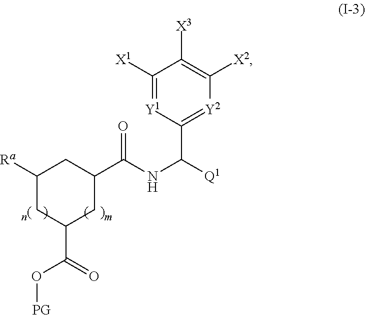

wherein
m is 0, or 1, and n is 0, 1, or 2, wherein m+n is an integer from 1 to 2;
Y¹ and Y² are each CH, or
one of Y¹ and Y² is N and the other of Y¹ and Y² is CH;
X¹ and X² are each independently H or halo;

X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1, provided that, when X³ is H, then Q¹ is C₆₋₁₀cycloalkyl, wherein the C₆₋₁₀cycloalkyl of Q¹ is optionally substituted with one or more C₁₋₆alkyl;
R^{a} is H, halo, —OH, or —NH—C(O)—C₁₋₆alkoxy; and
PG is a protecting group:
followed by,
(b) contacting the compound of formula (I-3) with deprotecting agent to provide a compound of formula (I).

In some embodiments, the protecting group is an alkyl protecting group. In some embodiments, the protecting group is a tert-butoxy group. In some embodiments, the protecting group is an allyl protecting group. In some embodiments, the protecting group is a propenyl group.

In some embodiments, the coupling reagent comprises EDCCI, TCFH, or T3P. In some embodiments, the process further comprises the presence of a base. In some embodiments, the base comprises an amine. In some embodiments, the amine is DMAP, NMM, or a trialkylamine. In some embodiments, the coupling reagent is N,N,N′,N′-tetramethylchloroformamidinium hexafluorophosphate (TFCH).

In some embodiments, the deprotecting agent comprises an acid. In some embodiments the acid is HCl, TFA, or barbituric acid. In some embodiments, the deprotecting agent comprises tetrakis(triphenylphosphine) palladium (0). In some embodiments, the deprotecting agent comprises tetrakis(triphenylphosphine) palladium (0) and barbituric acid.

In some embodiments, a process for preparing a compound of formula (I), or a stereoisomer or tautomer thereof, or a pharmaceutically acceptable salt of any of the foregoing, comprises:
reacting a compound of formula (1-1):

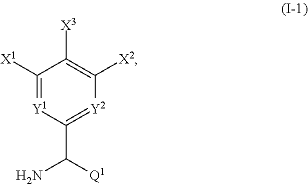

or a salt thereof, wherein
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl; and
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl;
with a compound of formula (I-4):

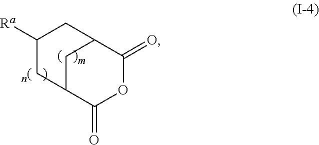

wherein,
m is 0, or 1, and n is 0, 1, or 2, wherein m+n is an integer from 1 to 2;
Y¹ and Y² are each CH, or one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more-NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl; and
R^{a} is H, halo, —OH, or —NH—C(O)—C₁₋₆alkoxy;
in the presence of a coupling reagent to provide a compound of formula (I).

In some embodiments, the coupling reagent comprises a base. In some embodiments, the base comprises an amine. In some embodiments, the base comprises a tertiary amine. In some embodiments, the amine is DIEA, or a trialkylamine.

### EXAMPLES

The following synthetic reaction schemes, which are detailed in the Schemes and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including, but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### SYNTHETIC EXAMPLES

As depicted in the Schemes and Examples below, in certain exemplary embodiments, compounds of formula (I), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

### Schemes

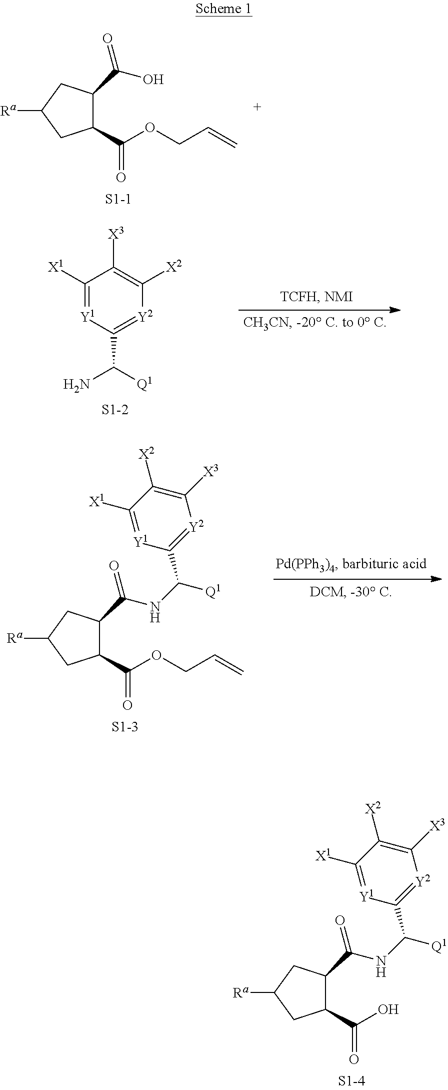

Compounds of formula S1-4 can be prepared as outlined in general Scheme 1. Amide coupling of a mono-protected carboxylic acid S1-1 with amine S1-2 using a coupling reagent such as N,N,N′,N′-tetramethylchloroformamidinium hexafluorophosphate (TFCH) and a base such as N-methylimidazole (NMI) in an aprotic solvent such as acetonitrile gives amide S1-3. Removal of the allyl protecting group with a metal catalyst such as tetrakis(triphenylphosphine) palladium (0) and barbituric acid in a solvent such as DCM gives compounds of formula S1-4. If desired, compounds of formula S1-4 may be further purified by chiral SFC.

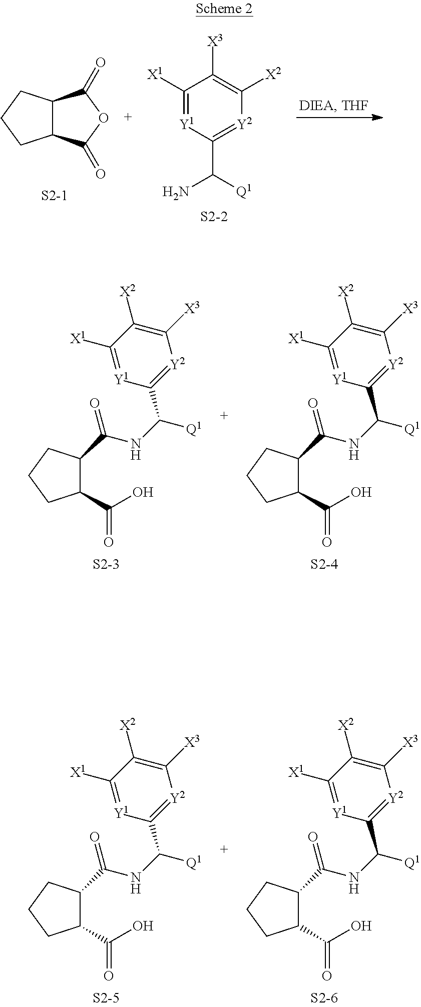

Compounds of formulae S2-3, S2-4, S2-5, and S2-6 can be prepared as outlined in general Scheme 2. Reaction of anhydride S2-1 with amine S2-2 using a tertiary amine base such as DIEA in an aprotic solvent such as THE gives rise to compounds of formulae S2-3, S2-4, S2-5, and S2-6. These compounds may be further purified using a method such as chiral SFC to provide compounds as single stereoisomers.

Abbreviations used are those conventional in the art and are in accordance with the Periodic Table of the Elements, CAS version, Handbook of Chemistry and Physics, 75th Ed. The following examples are intended to be illustrative only and not limiting in any way.

| | |
|---|---|
| ° C. | degrees Celsius |
| μL | microliter |
| [M + XX]⁺ | observed mass |
| AC₅₀ | half-maximal activity concentration |
| ACN | acetonitrile |
| app | apparent (NMR) |
| BH₃•THF | borane-tetrahydrofuran complex |
| BBr₃ | boron tribromide |
| Calc'd | calculated |
| Cbz-Cl | benzyl chloroformate |
| CO₂ | carbon dioxide |
| Cs₂CO₃ | cesium carbonate |
| d | deuterated (NMR solvents) |
| d | doublet (NMR) |
| dd | doublet of doublets (NMR) |
| DCM | dichloromethane |
| DIAD | diisopropyl azodicarboxylate |
| DMF | N,N-dimethylformamide |
| EC₅₀ | half-maximal effective concentration |
| EDCI | 1-Ethyl-3-(3-dimethylaminopropyl)carbodiimide |
| ESI | electrospray ionization |
| EtOAc | ethyl acetate |
| EtOH | ethanol |
| eq | equivalents |
| g | grams |
| h | hours |
| H | hydrogen |
| HCl | hydrochloric acid |
| HPLC | high-performance liquid chromatography |
| IC₅₀ | half-maximal inhibitory concentration |
| In vacuo | in a vacuum |
| IUPAC | International Union of Pure and Applied Chemistry |
| MHz | megahertz |
| J | J-coupling value (NMR) |
| K₂CO₃ | potassium carbonate |
| LDA | lithium diisopropylamide |
| LiHMDS | lithium bis(trimethylsilyl)amide |
| MeOH | Methanol |
| MeCN | acetonitrile |
| m | multiplet (NMR) |
| mg | milligrams |
| min | minutes |
| mL | milliliter |
| mmol | millimole |
| mM | millimolar |
| M | molarity or molar |
| MS | mass spectrometry |
| MsCl | methanesulfonyl chloride |
| MTBE | methyl tert-butyl ether |
| n/a | not applicable |
| NBS | N-bromosuccinimide |
| NH₄ | ammonium |
| NH₄OH | ammonium hydroxide |
| NH₄HCO₃ | ammonium bicarbonate |
| Na₂SO₄ | sodium sulfate |
| NaBH₃CN | sodium cyanoborohydride |
| NMI | N-methylimidazole |
| NMM | N-methylmorpholine |
| NMR | nuclear magnetic resonance |
| NaOH | sodium hydroxide |
| PCy₃ | Tricyclohexylphosphine |
| PdCl₂(dppf) | [1,1′-Bis(diphenylphosphino)ferrocene]dichloropalladium(II) |
| pH | potential of hydrogen |
| PPh₃s | triphenyl phosphine singlet (NMR) |
| SFC | super fluid chromatography |
| t | triplet (NMR) |
| T3P | Propanephosphonic acid anhydride |
| TBAB | tetrabutylammonium bromide |
| TEA | triethylamine |
| TFA | trifluoroacetic acid |
| TFCH | N,N,N′,N′-tetramethylchloroformamidinium hexafluorophosphate |
| THF | tetrahydrofuran |
| TMSCl | trimethylsilyl chloride |
| wt. % | weight percent |

### Intermediate A-1: Synthesis of (R)-(3-fluoro-4-isopropylphenyl)(1-methyl-2-oxo-2,3-dihydro-1H-benzo[d]imidazol-4-yl) methanaminium chloride

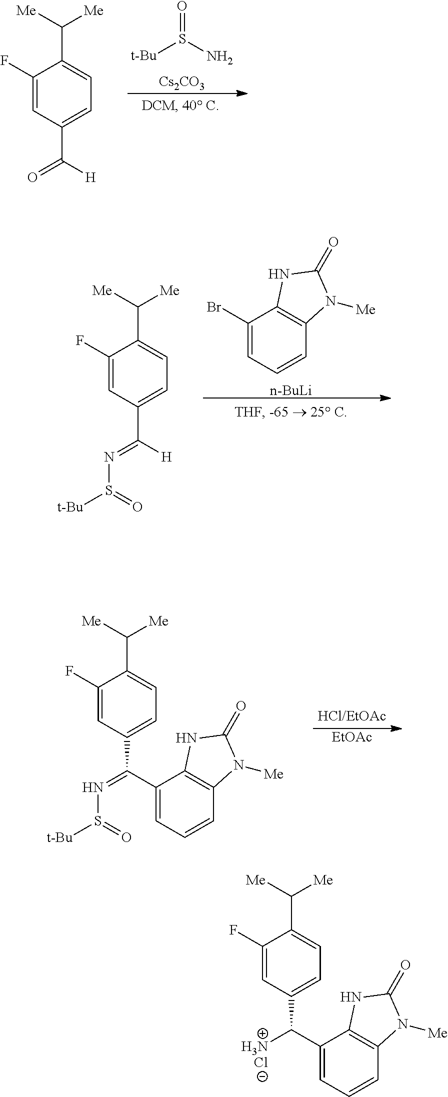

Step a: To a solution of 3-fluoro-4-isopropylbenzaldehyde (700 mg, 4.21 mmol, 1 eq) and 2-methylpropane-2-sulfinamide (816 mg, 6.74 mmol, 1.6 eq) in DCM (20 mL) was added Cs₂CO₃ (4.12 g, 12.6 mmol, 3 eq). The resulting mixture was then warmed to 40° C. and stirred for 2 h. The reaction mixture was then filtered and diluted with water, and the filtrate was extracted with DCM (3×20 mL). The combined organic extracts were washed with H₂O (30 mL) and brine (20 mL), dried over anhydrous Na₂SO₄, filtrated, and concentrated under reduced pressure. The crude residue obtained was purified by column chromatography to give (E)-N-(3-fluoro-4-isopropylbenzylidene)-2-methylpropane-2-sulfinamide. LC-MS (ESI): m/z: [M+H]⁺ calculated for C₁₄H₂₀FNOS: 270.1. found 270.2.

Step b: To a solution of 4-bromo-1-methyl-1,3-dihydro-2H-benzo[d]imidazol-2-one (1.00 g, 4.40 mmol, 1 eq) in THF (20 mL) at −65° C. under N₂ atmosphere was added n-BuLi (2.50 M in hexanes, 7.05 mL, 4 eq) in a dropwise manner. The resulting mixture was stirred for 3 h at −65° C. A solution of (E)-N-(3-fluoro-4-isopropylbenzylidene)-2-methylpropane-2-sulfinamide (2.37 g, 8.81 mmol, 2 eq) in THF (5 mL) was then added in a dropwise manner to the reaction mixture at −65° C. The resulting mixture was then allowed to warm to 25° C. and stirred at for 2 h. The reaction mixture was then cooled to 0° C. and quenched by addition of saturated aqueous NH₄Cl solution (20 mL). The resulting mixture was then allowed to warm to room temperature before it was diluted with water (10 mL). The resulting biphasic mixture was extracted with EtOAc (3×30 mL). The combined organic extracts were washed with brine (20 mL), dried over anhydrous Na₂SO₄, filtered, and concentrated under reduced pressure to give N-((3-fluoro-4-isopropylphenyl)(1-methyl-2-oxo-2,3-dihydro-1H-benzo[d]imidazol-4-yl)methyl)-2-methylpropane-2-sulfinamide as a mixture of isomers. These isomers were separated by prep-HPLC (column: Phenomenex Luna C18) to give N—((R)-(3-fluoro-4-isopropylphenyl)(1-methyl-2-oxo-2,3-dihydro-1H-benzo[d]imidazol-4-yl)methyl)-2-methylpropane-2-sulfinamide as the first eluting isomer. LC-MS (ESI): m/z: [M+H]⁺ calculated for C₂₂H₂₈FN₃O₂S: 418.2. found 418.2.

Step c: To a mixture of N—((R)-(3-fluoro-4-isopropylphenyl)(1-methyl-2-oxo-2,3-dihydro-1H-benzo[d]imidazol-4-yl)methyl)-2-methylpropane-2-sulfinamide (120 mg, 287 μmol, 1.00 eq) in EtOAc (1 mL) at 0° C. was added HCl/EtOAc (5 mL). The resulting mixture was then stirred at 0° C. for 1 h. The reaction mixture was then filtered, and the filter cake was washed with MTBE (3×5 mL). The solid obtained was dried under reduced pressure to give (R)-(3-fluoro-4-isopropylphenyl)(1-methyl-2-oxo-2,3-dihydro-1H-benzo[d]imidazol-4-yl) methanaminium chloride.

### Intermediate A-2: Synthesis of (3-fluoro-4-isopropylphenyl)(o-tolyl) methanaminium chloride

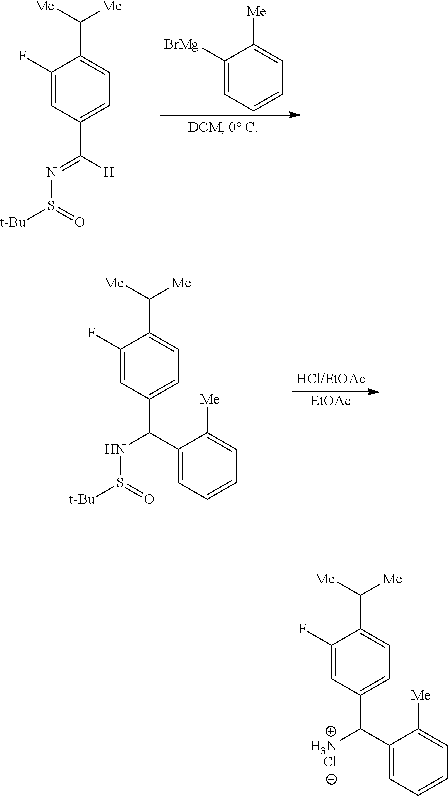

Step a: To a solution of (E)-N-(3-fluoro-4-isopropylbenzylidene)-2-methylpropane-2-sulfinamide (1.5 g, 5.57 mmol, 1 eq) in DCM (15 mL) at 0° C. under N₂ atmosphere was added o-tolylmagnesium bromide (0.9 M in diethyl ether, 15.4 mL, 2.5 eq) in a dropwise manner. The resulting mixture was warmed to 20° C. and stirred for 2 h. The reaction solution was then quenched with H₂O (30 mL), and the resulting biphasic mixture was extracted with EtOAc (3×30 mL). The organic extracts were combined and dried over anhydrous Na₂SO₄, filtered, and concentrated under reduced pressure to give N-((3-fluoro-4-isopropylphenyl)(o-tolyl)methyl)-2-methylpropane-2-sulfinamide. LC-MS (ESI): m/z: [M+H]⁺ calculated for C₂₁H₂₈FNOS: 362.2. found 362.2.

Step b: To a solution of N-((3-fluoro-4-isopropylphenyl)(o-tolyl)methyl)-2-methylpropane-2-sulfinamide (2.6 g, 7.19 mmol, 1 eq) in EtOAc (5 mL) at 0° C. was added HCl/EtOAc (10 mL). The resulting mixture was warmed to 20° C. and stirred for 1.5 h. The reaction was then concentrated under reduce pressure to give (3-fluoro-4-isopropylphenyl)(o-tolyl) methanaminium chloride. LC-MS (ESI): m/z: [M−H₃]⁺ calculated for C₁₇H₂₀FN: 241.1. found 241.2.

### Intermediate A-3: Synthesis of (6-fluoro-5-isopropylpyridin-2-yl)(1H-indazol-6-yl)methanaminium chloride

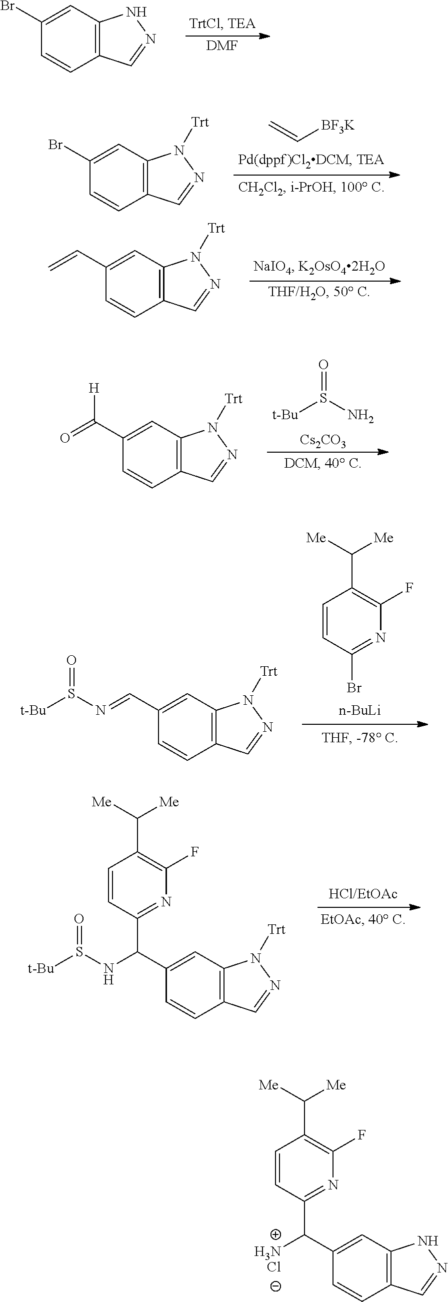

Step a: To a solution of 6-bromo-1H-indazole (8 g, 40.6 mmol, 1 eq) in DMF (50 mL) was added trityl chloride (TrtCl, 12.4 g, 44.6 mmol, 1.1 eq) and TEA (7.06 mL, 50.7 mmol, 1.25 eq). The resulting mixture was stirred at 25° C. for 16 h. The reaction mixture was then diluted with water, and the resulting biphasic mixture was extracted with ethyl acetate (3×50 mL). The combined organic extracts were dried over anhydrous Na₂SO₄, filtered, and concentrated under reduced pressure. The crude residue obtained was triturated with MTBE (30 mL) and filtered to give 6-bromo-1-trityl-1H-indazole, which was carried forward to the next step without further purification or characterization.

Step b: To a mixture of 6-bromo-1-trityl-1H-indazole (16.7 g, 38.0 mmol, 1 eq), potassium vinyltrifluoroborate (10.1 g, 76.0 mmol, 2 eq) and TEA (15.8 mL, 14.0 mmol, 3 eq) in i-PrOH (160 mL), was added Pd(dppf)Cl₂·CH₂Cl₂ (1.55 g, 1.90 mmol, 0.05 eq) under N₂. The resulting mixture was then degassed and placed under an N₂ atmosphere. The reaction mixture was then warmed to 100° C. and stirred for 2 h under N₂. After cooling, the mixture was filtered, and the filter cake was washed with ethyl acetate (3×100 mL). The combined filtrates were concentrated, and the crude residue obtained was purified by column chromatography to give 1-trityl-6-vinyl-1H-indazole. LC-MS (ESI): m/z: [2M+Na]⁺ calculated for C₂₈H₂₂N₂: 795.4. found 795.3.

Step c: To a solution of 1-trityl-6-vinyl-1H-indazole (14.2 g, 36.7 mmol, 1 eq) in THF:H₂O (5:1)(300 mL) at 0° C. was added NaIO₄ (31.4 g, 146 mmol, 4 eq) and K₂OsO₄·2H₂O (676 mg, 1.84 mmol, 0.05 eq). The resulting mixture was warmed to 50° C. and stirred for 1 h. The reaction mixture was then cooled to 25° C. and quenched with sat. aq. Na₂S₂O₃ (100 mL). The resulting mixture was extracted with ethyl acetate (3×100 mL), and the combined extracts were dried over Na₂SO₄, filtered, and concentrated under reduced pressure. The resulting residue was purified by column chromatography to give 1-trityl-1H-indazole-6-carbaldehyde.

Step d: To a solution 1-trityl-1H-indazole-6-carbaldehyde (7.3 g, 18.8 mmol, 1 eq) in DCM (75 mL) was added Cs₂CO₃ (6.74 g, 20.7 mmol, 1.1 eq) and 2-methylpropane-2-sulfinamide (2.51 g, 20.6 mmol, 1.1 eq). The mixture was then warmed to 40° C. and stirred for 16 h. The reaction mixture was then filtered, and the filter cake was washed with ethyl acetate (3×100 mL). The filtrate was then filtered and concentrated under reduced pressure. The crude residue obtained was purified by column chromatography to give (E)-2-methyl-N-((1-trityl-1H-indazol-6-yl)methylene) propane-2-sulfinamide.

Step e: To a solution of 6-bromo-2-fluoro-3-isopropylpyridine (665 mg, 3.05 mmol, 1.5 eq) in THF (5 mL) at −78° C. under N₂ was added n-BuLi (1.22 mL, 2.5 M, 1.5 eq). The resulting mixture was stirred at −78° C. for 0.5 h. After this time, (E)-2-methyl-N-((1-trityl-1H-indazol-6-yl)methylene) propane-2-sulfinamide (1 g, 2.03 mmol, 1 eq) in THF (5 mL) cooled to −78° C. under N₂ was added, and the resulting mixture was stirred at −78° C. for 4 h. The reaction was then quenched with saturated aqueous NH₄Cl (20 mL), and the resulting biphasic mixture was extracted with ethyl acetate (3×20 mL), dried over anhydrous Na₂SO₄, filtered, and concentrated under reduced pressure. The crude residue obtained was purified by column chromatography to give N-((6-fluoro-5-isopropylpyridin-2-yl)(1-trityl-1H-indazol-6-yl)methyl)-2-methylpropane-2-sulfinamide.

Step f: To a solution of N-((6-fluoro-5-isopropylpyridin-2-yl)(1-trityl-1H-indazol-6-yl)methyl)-2-methylpropane-2-sulfinamide (600 mg, 951 μmol, 1 eq) in EtOAc (3 mL) at 0° C. was added HCl/EtOAc (4 M, 3 mL, 12.6 eq). The resulting mixture was then warmed to 40° C. and stirred for 16 h. The reaction mixture was then filtered and concentrated to give (6-fluoro-5-isopropylpyridin-2-yl)(1H-indazol-6-yl) methanaminium chloride. LC-MS (ESI): m/z: [M−NH₃]⁺ calculated for C₁₆H₁₇FN₄: 268.1. found 268.2.

### Intermediate A-4: Synthesis of (3-(1H-pyrazol-5-yl)phenyl)(6-fluoro-5-isopropylpyridin-2-yl)methanaminium chloride

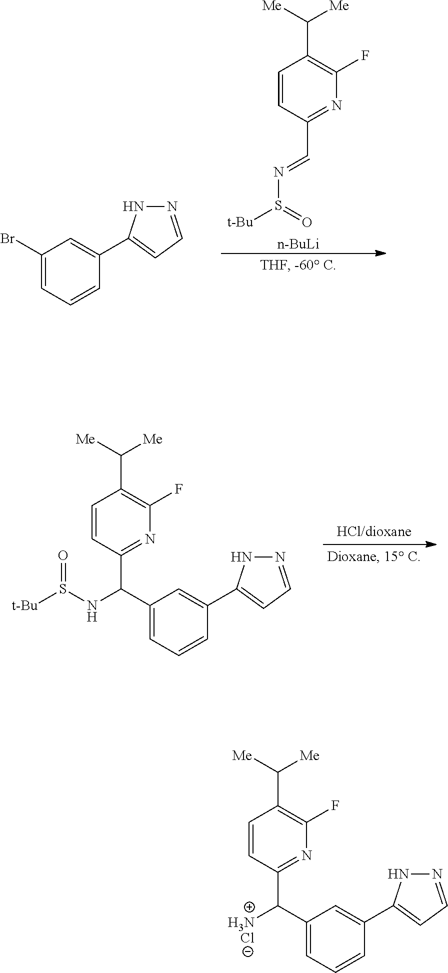

Step a: To a solution of 5-(3-bromophenyl)-1H-pyrazole (408 mg, 1.83 mmol, 1.5 eq) in THF (3 mL) at −60° C. under N₂ was added n-BuLi (2.5 M, 1.22 mL, 2.5 eq) in a dropwise manner. Once this addition was complete, (E)-N-((6-fluoro-5-isopropylpyridin-2-yl)methylene)-2-methylpropane-2-sulfinamide (330 mg, 1.22 mmol, 1 eq) in THF (2 mL) was added in a dropwise manner. The resulting mixture was stirred at −60° C. for 2 h. The reaction mixture was then poured into ice-water (30 mL) and stirred for 2 min. The resulting biphasic mixture was then extracted with ethyl acetate (3×20 mL). The combined organic extracts were washed with brine (30 mL), dried over anhydrous Na₂SO₄, filtered, and concentrated under reduced pressure. The crude residue obtained was purified by column chromatography to give N-((3-(1H-pyrazol-5-yl)phenyl)(6-fluoro-5-isopropylpyridin-2-yl)methyl)-2-methylpropane-2-sulfinamide. LC-MS (ESI): m/z: [M+H]⁺ calculated for C₂₂H₂₆FNOS: 415.2. found 415.2.

Step b: To a solution of N-((3-(1H-pyrazol-5-yl)phenyl)(6-fluoro-5-isopropylpyridin-2-yl)methyl)-2-methylpropane-2-sulfinamide (410 mg, 989 μmol, 1 eq) in dioxane (2 mL) at 15° C. was added HCl/dioxane (4 mL) in a dropwise manner. The resulting mixture was stirred at 15° C. for 2 h. The reaction mixture was then concentrated under reduced pressure to give (3-(1H-pyrazol-5-yl)phenyl)(6-fluoro-5-isopropylpyridin-2-yl) methanaminium chloride. LC-MS (ESI): m/z: [M+H]⁺ calculated for C₁₈H₁₉FN₄: 311.2. found 311.2.

### Intermediate A-5: Synthesis of(S)-(3-fluoro-4-isopropylphenyl)(phenyl) methanaminium chloride

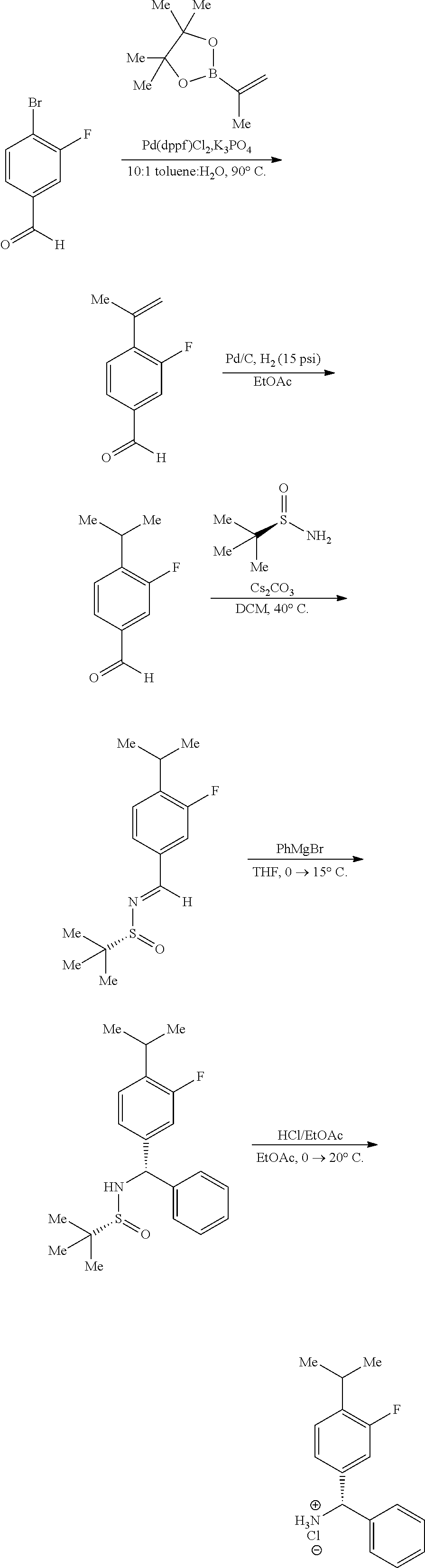

Step a: To a mixture of 4-bromo-3-fluoro-benzaldehyde (200 g, 985 mmol, 1.00 eq) and 2-isopropenyl-4,4,5,5-tetramethyl-1,3,2-dioxaborolane (215 g, 1.28 mol, 1.30 eq) in toluene (3.70 L) and H₂O (410 mL) at 25° C. under N₂ was added Pd (dppf)Cl₂ (36.0 g, 49.3 mmol, 0.05 eq) and K₃PO₄ (418 g, 1.97 mol, 2.00 eq). The mixture was warmed to 90° C. and stirred for 12 h. The reaction mixture was then filtered, and the filtrate was concentrated under reduced pressure. The resulting crude residue was purified by column chromatography to give 3-fluoro-4-isopropenyl-benzaldehyde. The compound was carried forward to the next step without further characterization.

Step b: To a solution of 3-fluoro-4-isopropenyl-benzaldehyde (124 g, 755 mmol, 1.00 eq) in EtOAc (1.20 L) under N₂ was added Pd/C (85.0 g, 10 wt. %). The suspension was degassed and purged with H₂ several times. The mixture was stirred at 25° C. under H₂ (15 psi) for 1 h. The reaction mixture was then filtered, and the filtrate was concentrated under reduced pressure. The resulting crude residue was purified by column chromatography to give 3-fluoro-4-isopropyl-benzaldehyde. The compound was carried forward to the next step without further characterization.

Step c: To a mixture of 3-fluoro-4-isopropyl-benzaldehyde (80.0 g, 481 mmol, 1.00 eq) and (R)-2-methylpropane-2-sulfinamide (64.2 g, 523 mmol, 1.10 eq) in DCM (450 mL) at 25° C. was added Cs₂CO₃ (173 g, 530 mmol, 1.10 eq). The mixture was warmed to 40° C. and stirred for 16 h. The reaction mixture was filtered, and the filtrate was concentrated under reduced pressure. The residue was purified by column chromatography to obtain (R,E)-N-(3-fluoro-4-isopropylbenzylidene)-2-methylpropane-2-sulfinamide. LC-MS (ESI): m/z: [M+H]⁺ calculated for C₁₄H₂₀FNOS: 270.1. found 270.0.

Step d: To a solution of (R,E)-N-(3-fluoro-4-isopropylbenzylidene)-2-methylpropane-2-sulfinamide (30.0 g, 111 mmol, 1.00 eq) in THF (400 mL)−65° C. under N₂ was added, dropwise, a solution of phenylmagnesium bromide (3 M in Et₂O, 55.7 mL, 1.50 eq) over a period of 30 min. The reaction mixture was stirred at −65° C. for 6 h, then warmed to 25° C. and stirred for an additional 6 h. The reaction mixture was quenched with saturated aqueous NH₄Cl (50 mL) and extracted with EtOAc (3×30 mL). The combined organic extracts were washed with water (3×30 mL), dried over anhydrous Na₂SO₄, filtered, and concentrated under reduced pressure. The resulting crude residue was purified by column chromatography to obtain (R)—N—((S)-(3-fluoro-4-isopropylphenyl)(phenyl)methyl)-2-methylpropane-2-sulfinamide. The compound was carried forward to the next step without further characterization.

Step e: To a mixture of (R)—N—((S)-(3-fluoro-4-isopropylphenyl)(phenyl)methyl)-2-methylpropane-2-sulfinamide (35.0 g, 101 mmol, 1.00 eq) in EtOAc (300 mL) at 25° C. was added HCl/EtOAc (4 M, 50.4 mL, 2.00 eq), and the mixture was stirred for 2 h. The reaction mixture was filtered and the solid so obtained was set aside. The filtrate was concentrated under reduced pressure and the resulting residue was combined with the previously obtained solid. The mixture was dissolved in MTBE (200 mL) and filtered, and the filtrate was concentrated under reduced pressure to give(S)-(3-fluoro-4-isopropylphenyl)(phenyl) methanaminium chloride.

### Intermediate A-6: Synthesis of(S)-(5-cyclopropyl-6-fluoropyridin-2-yl)(phenyl)methanaminium chloride

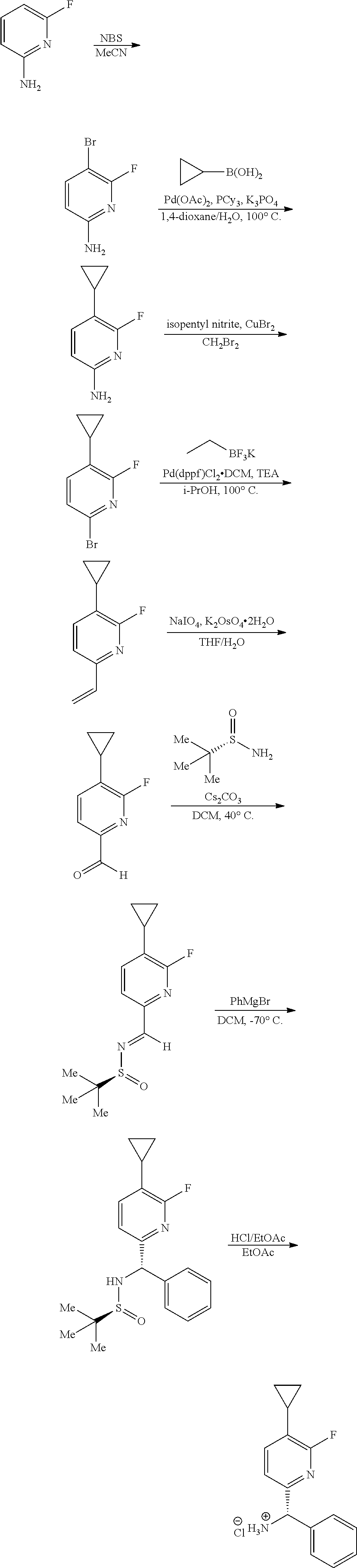

Step a: In four parallel reactions, 6-fluoropyridin-2-amine (125 g, 1.11 mol, 1 eq) in MeCN (1.2 L) at 0° C. under N₂ was treated with NBS (209 g, 1.17 mmol, 1.05 eq) in MeCN (1.2 L). The reaction mixtures were stirred at 20° C. for 2 h. The four parallel reactions were combined, and the resulting mixture was concentrated under reduced pressure. The resulting crude residue was purified by column chromatography to give 5-bromo-6-fluoropyridin-2-amine. LC-MS (ESI): m/z: [M+H]⁺ calculated for C₅H₄BrFN₂: 190.9. found 191.0.

Step b: To a mixture of 5-bromo-6-fluoropyridin-2-amine (200 g, 1.04 mol, 1 eq) and cyclopropylboronic acid (226 g, 2.63 mol, 2.5 eq) in 1,4-dioxane (2 L) and H₂O (200 mL) under N₂ were added K₃PO₄ (666 g, 3.14 mol, 3 eq), PCy₃ (58.6 g, 209 mmol, 0.2 eq), and Pd(OAc)₂ (11.7 g, 52.3 mmol, 0.05 eq). The system was then degassed and charged with nitrogen three times. The reaction mixture was warmed to 100° C. and stirred for 12 h. The reaction mixture was then cooled to room temperature and filtered through Celite. The resulting filtrate was diluted with H₂O (2 L) and then extracted with EtOAc (3×500 mL). The combined organic extracts were washed with brine (2×300 mL), dried over anhydrous Na₂SO₄, filtered, and concentrated under reduced pressure. The crude residue obtained was purified by column chromatography to give 5-cyclopropyl-6-fluoropyridin-2-amine. LC-MS (ESI): m/z: [M+H]⁺ calculated for C₈H₉FN₂: 153.1. found 153.0.

Step c: To a mixture of 5-cyclopropyl-6-fluoropyridin-2-amine (120 g, 788 mmol, 1 eq) in dibromomethane (564 mL) under N₂ was added isopentyl nitrite (110 g, 946 mmol, 127 mL, 1.2 eq). To the resulting mixture was added CuBr₂ (211 g, 946 mmol, 44.3 mL, 1.2 eq) over 0.5 h. The final mixture was then degassed and charged with nitrogen three times before stirring at 20° C. for 16 h. The reaction mixture was then filtered, and the filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was purified by column chromatography to give 6-bromo-3-cyclopropyl-2-fluoropyridine. LC-MS (ESI): m/z: [M+H]⁺ calculated for C₈H₇BrFN; 216.0. found 216.1.

Step d: To a mixture of 6-bromo-3-cyclopropyl-2-fluoropyridine (90 g, 416 mmol, 1 eq) and trifluoro (vinyl)-24-borane, potassium salt (83.7 g, 624 mmol, 1.5 eq) in i-PrOH (900 mL) at 20° C. under N₂ was added TEA (126 g, 1.25 mol, 3 eq) and Pd(dppf)Cl₂·DCM (17 g, 20.8 mmol, 0.05 eq). The resulting mixture was degassed and charged with nitrogen three times. The reaction mixture was then warmed to 100° C. and stirred for 2 h. The reaction mixture was then cooled to room temperature and filtered. The filtrate was diluted with H₂O (500 mL) and extracted with EtOAc (3×300 mL). The combined organic extracts were washed with brine (300 mL), dried over anhydrous Na₂SO₄, filtered, and concentrated under reduced pressure. The crude residue obtained was then purified by column chromatography to give 3-cyclopropyl-2-fluoro-6-vinylpyridine. LC-MS (ESI): m/z: [M+H]⁺ calculated for C₁₀H₁₀FN: 164.1. found 164.1.

Step e: To a mixture of 3-cyclopropyl-2-fluoro-6-vinylpyridine (47 g, 288 mmol, 1 eq) in THF (800 mL) and H₂O (160 mL) at 20° C. under N₂ was added NaIO₄ (246 g, 1.15 mol, 4 eq) and K₂OsO₄·2H₂O (2.12 g, 5.76 mmol, 0.02 eq). The resulting mixture was degassed and charged with nitrogen three times before stirring for 2 h. The reaction mixture was then filtered, and the filtrate was diluted with H₂O (500 mL), and extracted with EtOAc (3×300 mL). The combined organic extracts were washed with brine (300 mL), dried over anhydrous Na₂SO₄, filtered, and concentrated under reduced pressure. The resulting crude residue was purified by column chromatography to give 5-cyclopropyl-6-fluoropicolinaldehyde. LC-MS (ESI): m/z: [M+H]⁺ calculated for C₉H₈FNO: 166.1. found 166.2.

Step f: To a mixture of 5-cyclopropyl-6-fluoropicolinaldehyde (38 g, 230 mmol, 1 eq) and(S)-2-methylpropane-2-sulfinamide (30.6 g, 253 mmol, 1.1 eq) in DCM (200 mL) at 20° C. under N₂ was added Cs₂CO₃ (82.4 g, 253 mmol, 1.1 eq). The system was then degassed and charged with nitrogen three times. The resulting mixture was then warmed to 40° C. and stirred for 12 h. The reaction solution was then diluted with H₂O (300 mL) and extracted with DCM (3×200 mL). The combined organic extracts were washed with brine (200 mL), dried over anhydrous Na₂SO₄, filtered, and concentrated under reduced pressure. The resulting crude residue was then purified by column chromatography to give (S,E)-N-((5-cyclopropyl-6-fluoropyridin-2-yl)methylene)-2-methylpropane-2-sulfinamide. LC-MS (ESI): m/z: [M+H]⁺ calculated for C₁₃H₁₇FN₂OS: 269.1. found 269.2.

Step g: To a solution of (S,E)-N-((5-cyclopropyl-6-fluoropyridin-2-yl)methylene)-2-methylpropane-2-sulfinamide (58 g, 216 mmol, 1 eq) in dry DCM (600 mL) at −70° C. under nitrogen was added PhMgBr (3 M in Et₂O, 93.6 mL, 281 mmol, 1.3 eq) in a dropwise manner. The resulting reaction mixture was stirred at −70° C. for 1 h. The reaction mixture was then quenched with saturated aqueous NH₄Cl solution (500 mL), warmed to room temperature, and extracted with EtOAc (3×200 mL). The combined organic extracts were washed with brine (200 mL), dried over anhydrous Na₂SO₄, filtered, and concentrated under reduced pressure. The crude residue obtained was purified by column chromatography to give (S)—N—((S)-(5-cyclopropyl-6-fluoropyridin-2-yl)(phenyl)methyl)-2-methylpropane-2-sulfinamide. LC-MS (ESI): m/z: [M+H]⁺ calculated for C₁₉H₂₃FN₂OS: 347.2. found 347.3.

Step h: To a solution of(S)—N—((S)-(5-cyclopropyl-6-fluoropyridin-2-yl)(phenyl)methyl)-2-methylpropane-2-sulfinamide (74 g, 213 mmol, 1 eq) in EtOAc (100 mL) at 0° C. under N₂ was added HCl/EtOAc (4 M, 740 mL, 2940 mmol, 13.8 eq). The resulting mixture was then warmed 20° C. and stirred for 1 h. The reaction mixture was then concentrated under reduced pressure, and the crude residue obtained was triturated with MTBE (500 mL). The resulting solid was collected by filtration and dried under reduced pressure to give(S)-(5-cyclopropyl-6-fluoropyridin-2-yl)(phenyl) methanaminium chloride. LC-MS (ESI): m/z: [M+H]⁺ calculated for C₁₅H₁₅FN₂: 243.1. found 243.2.

### Intermediate A-7: Synthesis of(S)-(6-fluoro-5-isopropylpyridin-2-yl)(3-(1-methyl-1H-pyrazol-5-yl)phenyl) methanaminium chloride

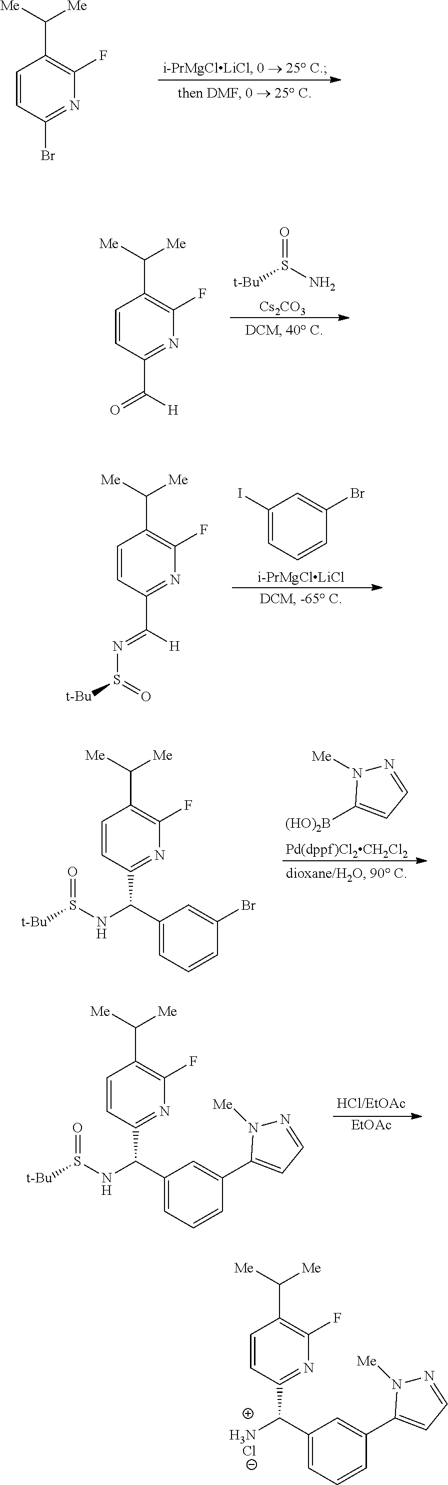

Step a: In two parallel reactions, a solution of 6-bromo-2-fluoro-3-isopropylpyridine (5 g, 22.9 mmol, 1 eq) in THF (25 mL) was cooled to 0° C. under N₂. To this solution was added i-PrMgCl·LiCl (1.3 M in THF, 26.5 mL, 1.5 eq) in a dropwise manner. The reaction mixture was then allowed to warm to 25° C. and stirred for 2 h. At this time, the reaction mixture was cooled to 0° C., and DMF (5.3 mL, 68.8 mmol, 3 eq) was added in a dropwise manner. After the addition was complete, the reaction mixture was allowed to warm to 25° C. and stirred for 1 h. At this time, the two separate reactions were combined for work up. The combined reaction mixture was quenched with NH₄Cl (70 mL), and the resulting biphasic mixture was extracted with ethyl acetate (2×50 mL). The combined organic extracts were washed with brine (2×50 mL), dried over anhydrous Na₂SO₄, filtered, and concentrated under reduced pressure. The crude residue obtained was purified by column chromatography to give 6-fluoro-5-isopropylpicolinaldehyde. LC-MS (ESI): m/z: [M+H]⁺ calculated for C₉H₁₀FNO: 168.1. found 168.2.

Step b: In two parallel reactions, Cs₂CO₃ (3.64 g, 11.2 mmol, 1.1 eq) was added to a mixture of 6-fluoro-5-isopropylpicolinaldehyde (1.70 g, 10.1 mmol, 1 eq) and(S)-2-methylpropane-2-sulfinamide (1.36 g, 11.2 mmol, 1.1 eq) in DCM (20 mL). The resulting mixture was warmed to 40° C. under N₂ and stirred for 2 h. At this time, the two parallel reactions were concentrated under reduced pressure. The crude residue obtained was purified by column chromatography to give (S,E)-N-((6-fluoro-5-isopropylpyridin-2-yl)methylene)-2-methylpropane-2-sulfinamide. LC-MS (ESI): m/z: [M+H]⁺ calculated for C₁₃H₁₉FN₂OS: 271.1. found 271.1.

Step c: To a solution of 1-bromo-3-iodo-benzene (5.22 g, 18.5 mmol, 2 eq) in THF (8 mL) at 0° C. under N₂ was added i-PrMgCl· LiCl (1.3 M in THF, 10 mL, 1.4 eq) in a dropwise manner. The resulting mixture was warmed to 25° C. and stirred for 2 h. At this time, the reaction mixture was cooled to −65° C., and a solution of (S,E)-N-((6-fluoro-5-isopropylpyridin-2-yl)methylene)-2-methylpropane-2-sulfinamide (2.5 g, 9.25 mmol, 1 eq) in DCM (30 mL) was added in a dropwise manner. The resulting mixture was the stirred at −65° C. for 3 h under N₂. The reaction mixture was then quenched with NH₄Cl (70 mL), and the resulting biphasic mixture was extracted with ethyl acetate (2×100 mL). The combined organic extracts were washed with brine (2×50 mL), dried over anhydrous Na₂SO₄, filtered, and concentrated under reduced pressure. The crude residue obtained was purified by column chromatography. This material was further purified by prep-HPLC (column: Phenomenex Titank C18) to give(S)—N—((S)-(3-bromophenyl)(6-fluoro-5-isopropylpyridin-2-yl)methyl)-2-methylpropane-2-sulfinamide as the second eluting isomer. LC-MS (ESI): m/z: [M+H]⁺ calculated for C₁₉H₂₄BrFN₂OS: 427.1. found 427.0.

Step d: To a mixture of (S)—N—((S)-(3-bromophenyl)(6-fluoro-5-isopropylpyridin-2-yl)methyl)-2-methylpropane-2-sulfinamide (550 mg, 1.29 mmol, 1 eq) and (1-methyl-1H-pyrazol-5-yl) boronic acid (324 mg, 2.57 mmol, 2 eq) in dioxane (2.5 mL) and H₂O (2.5 mL) was added K₂CO₃ (534 mg, 3.86 mmol, 3 eq) and Pd(dppf)Cl₂·CH₂Cl₂ (105 mg, 129 μmol, 0.1 eq). The resulting mixture was then warmed to 90° C. and stirred for 2 h. The reaction mixture was then quenched with H₂O (10 mL) and extracted with ethyl acetate (2×10 mL). The combined organic extracts were washed with brine (20 mL), dried over anhydrous Na₂SO₄, filtered, and concentrated under reduced pressure. The crude residue obtained was purified by column to give(S)—N—((S)-(6-fluoro-5-isopropylpyridin-2-yl)(3-(1-methyl-1H-pyrazol-5-yl)phenyl)methyl)-2-methylpropane-2-sulfinamide. LC-MS (ESI): m/z: [M+H]⁺ calculated for C₂₃H₂₉FN₄OS: 429.2. found 429.3.

Step e: To a solution of(S)—N—((S)-(6-fluoro-5-isopropylpyridin-2-yl)(3-(1-methyl-1H-pyrazol-5-yl)phenyl)methyl)-2-methylpropane-2-sulfinamide in ethyl acetate (1 mL) at 0° C. was added HCl/EtOAc (4 M, 15 mL), and the resulting mixture was stirred at 0° C. for 1 h. The reaction was then concentrated under reduced pressure. The crude residue obtained was triturated with MTBE (10 mL) to give(S)-(6-fluoro-5-isopropylpyridin-2-yl)(3-(1-methyl-1H-pyrazol-5-yl)phenyl) methanaminium chloride. LC-MS (ESI): m/z: [M+H]⁺ calculated for C₁₉H₂₁FN₄: 325.2. found 325.2.

The following compounds in Table B-1 were synthesized using procedures similar to Intermediates A-1 through A-7 using the appropriate starting materials and reagents.

TABLE B-1

| | | | Exact | |
|---|---|---|---|---|
| Intermediate | | | mass | LCMS, Found |
| No. | Structure | IUPAC | (g/mol) | [M + H]⁺ |
| B-1-1 | 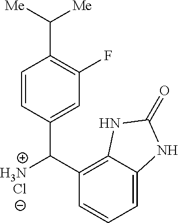 | (3-fluoro-4-isopropylphenyl)(2- oxo-2,3-dihydro-1H- benzo[d]imidazol-4- yl)methanaminium chloride | 299.1 | 282.9 [M − NH₃]⁺ |
| B-1-2 | 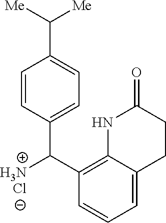 | (4-isopropylphenyl)(2-oxo- 1,2,3,4-tetrahydroquinolin-8- yl)methanaminium chloride | 294.2 | 278.3 [M − NH₃]⁺ |
| B-1-3 | 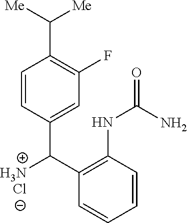 | (3-fluoro-4-isopropylphenyl)(2- uriedophenyl) methanaminium chloride | 301.2 | |
| B-1-4 | 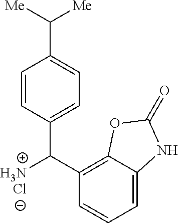 | (4-isopropylphenyl)(2-oxo-2,3- dihydrobenzo[d]oxazol-7- yl)methanaminium chloride | 282.1 | 266.2 [M − NH₃]⁺ |
| B-1-5 | 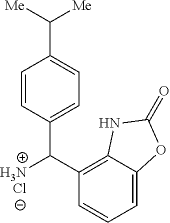 | (4-isopropylphenyl)(2-oxo-2,3- dihydrobenzo[d]oxazol-4- yl)methanaminium chloride | 282.1 | 266.1 [M − NH₃]⁺ |
| B-1-6 | 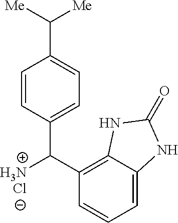 | (4-isopropylphenyl)(2-oxo-2,3- dihydro-1H-benzo[d]imidazol- 4-yl)methanaminium chloride | 281.1 | 265.1 [M − NH₃]⁺ |
| B-1-7 | 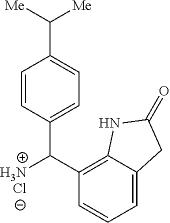 | (4-isopropylphenyl)(2- oxoindolin-7- yl)methanaminium chloride | 280.2 | 264.3 [M − NH₃]⁺ |
| B-1-8 | 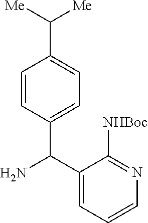 [SMILES: CC(C)c1ccc(C(N)c2cccnc2NC(=O)OC(C)(C)C)cc1] | tert-butyl (3-(amino(4- isopropylphenyl)methyl)pyridin- 2-yl)carbamate | 341.2 | 342.2 |
| B-1-9 | 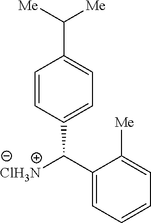 | (R)-(4-isopropylphenyl)(o- tolyl)methanaminium chloride | 239.2 | 223.2 [M − NH₃]⁺ |
| B-1-10 | 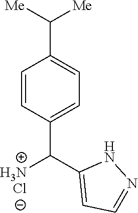 | (4-isopropylphenyl)(1H- pyrazol-5-yl)methanaminium chloride | 215.1 | 199.1 [M − NH₃]⁺ |
| B-1-11 | 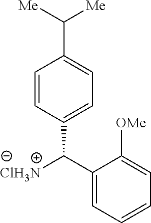 | (R)-(4-isopropylphenyl)(2- methoxyphenyl)methanaminium chloride | 255.2 | 239.1 [M − NH₃]⁺ |
| B-1-12 | 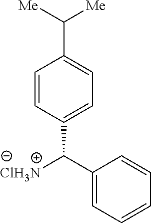 | (S)-(4- isopropylphenyl)(phenyl) methanaminium chloride | 225.1 | 209.1 [M − NH₃]⁺ |
| B-1-13 | 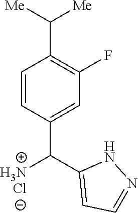 | (3-fluoro-4- isopropylphenyl)(1H-pyrazol- 5-yl)methanaminium chloride | 233.1 | 217.1 [M − NH₃]⁺ |
| B-1-14 | 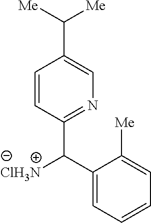 | (5-isopropylpyridin-2-yl)(o- tolyl)methanaminium chloride | 240.2 | 241.1 |
| B-1-15 | 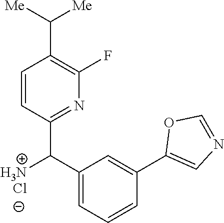 | (6-fluoro-5-isopropylpyridin-2- yl)(3-(oxazol-5- yl)phenyl)methanaminium chloride | 311.1 | 294.9 [M − NH₃]⁺ |
| B-1-16 | 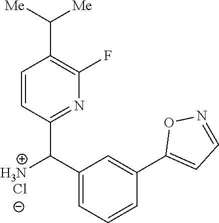 | (6-fluoro-5-isopropylpyridin-2- yl)(3-(isoxazol-5- yl)phenyl)methanaminium chloride | 311.1 | 312.2 |
| B-1-17 | 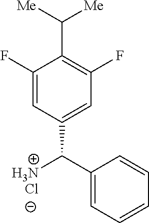 | (S)-(3,5-difluoro-4- isopropylphenyl)(phenyl) methanaminium chloride | 261.1 | |
| B-1-18 | 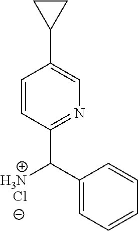 | (5-cyclopropylpyridin-2- yl)(phenyl)methanaminium chloride | 224.1 | 225.1 |
| B-1-19 | 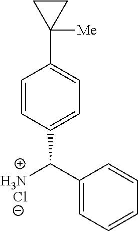 | (S)-(4-(1- methylcyclopropyl)phenyl) (phenyl)methanaminium chloride | 237.1 | |
| B-1-20 | 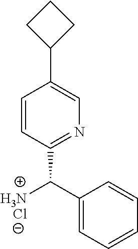 | (5-cyclobutylpyridin-2- yl)(phenyl)methanaminium chloride | 238.1 | |
| B-1-21 | 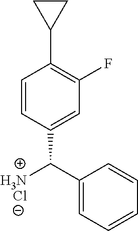 | (S)-(4-cyclopropyl-3- fluorophenyl)(phenyl) methanaminium chloride | 241.1 | |
| B-1-22 | 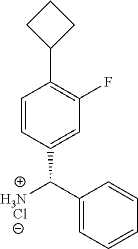 | (S)-(4-cyclobutyl-3- fluorophenyl)(phenyl) methanaminium chloride | 255.1 | |
| B-1-23 | 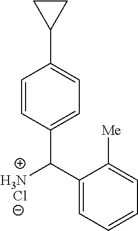 | (4-cyclopropylphenyl)(o- tolyl)methanaminium chloride | 237.1 | |
| B-1-24 | 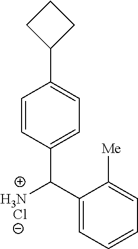 | (4-cyclobutylphenyl)(o- tolyl)methanaminium chloride | 251.2 | |
| B-1-25 | 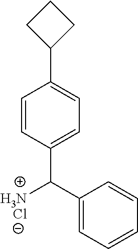 | (4- cyclobutylphenyl)(phenyl) methanaminium chloride | 237.1 | |
| B-1-26 | 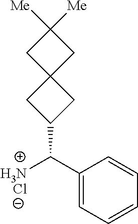 | (R)-(6,6- dimethylspiro[3.3]heptan-2- yl)(phenyl)methanaminium chloride | 229.2 | |
| B-1-27 | 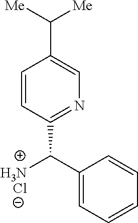 | (S)-(5-isopropylpyridin-2- yl)(phenyl)methanaminium chloride | 226.1 | 227.2 |
| B-1-28 | 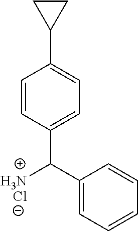 | (4- cyclopropylphenyl)(phenyl) methanaminium chloride | 223.1 | |
| B-1-29 | 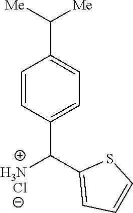 | (4-isopropylphenyl)(thiophen- 2-yl)methanaminium chloride | 231.1 | |
| B-1-30 | 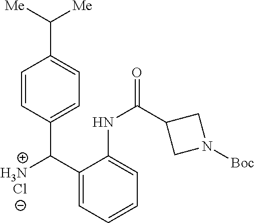 | (2-(1-(tert- butoxycarbonyl)azetidine-3- carboxamido)phenyl)(4- isopropylphenyl) methanaminium chloride | 423.2 | 424.2 |

### Intermediate A-9: Synthesis of (1R,2S)-2-((allyloxy)carbonyl)cyclopentane-1-carboxylic acid

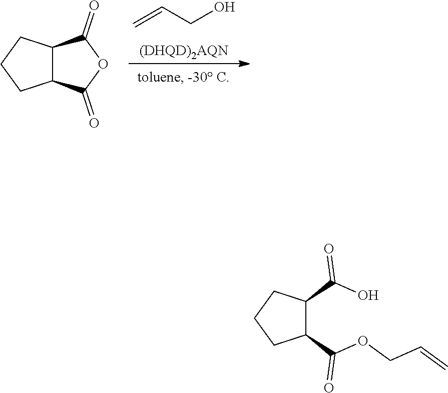

Step a: To a mixture of cis-tetrahydro-1H-cyclopenta[c]furan-1,3(3aH)-dione (6.25 g, 44.6 mmol, 1 eq) and (DHQD)₂AQN (CAS: 176298-44-5, 3.06 g, 3.57 mmol, 0.08 eq) in toluene (1.7 L) at −30° C. was added allyl alcohol (25.9 g, 446 mmol, 30 mL, 10 eq). The resulting mixture was stirred at −30° C. for 170 h. The reaction was then quenched by addition of aqueous HCl (1 M, 1 L), and the resulting biphasic mixture was extracted with EtOAc (2×1 L). The organic extracts were combined, dried over anhydrous Na₂SO₄, filtered, and concentrated under reduced pressure. The crude residue obtained was purified by column chromatography to give (1R,2S)-2-((allyloxy) carbonyl)cyclopentane-1-carboxylic acid.

### Example S-1

TABLE 2

| | | Exact | Observed |
|---|---|---|---|
| Cmpd | | Mass | Mass |
| No. | Cmpd Name | (g/mol) | (M + H) |
| 1 | (1S,2R)-2-(((S)-(6-fluoro-5-isopropylpyridin-2-yl)(3-(1- | 464.2 | 465.1 |
| | methyl-1H-pyrazol-5- | | |
| | yl)phenyl)methyl)carbamoyl)cyclopentane-1-carboxylic acid | | |
| 2 | (1S,2R)-2-(((S)-(6-fluoro-5-isopropylpyridin-2-yl)(3-(oxazol- | 451.2 | 452.1 |
| | 5-yl)phenyl)methyl)carbamoyl)cyclopentane-1-carboxylic | | |
| | acid | | |
| 3 | (1S,2R)-2-(((S)-(6-fluoro-5-isopropylpyridin-2-yl)(3- | 451.2 | 452.2 |
| | (isoxazol-5-yl)phenyl)methyl)carbamoyl)cyclopentane-1- | | |
| | carboxylic acid | | |
| 4 | (1S,2R)-2-(((S)-(3-(1H-pyrazol-5-yl)phenyl)(6-fluoro-5- | 450.2 | 451.1 |
| | isopropylpyridin-2-yl)methyl)carbamoyl)cyclopentane-1- | | |
| | carboxylic acid | | |
| 5 | (1S,2R)-2-(((S)-(6-fluoro-5-isopropylpyridin-2-yl)(1H- | 424.2 | 425.1 |
| | indazol-6-yl)methyl)carbamoyl)cyclopentane-1-carboxylic | | |
| | acid | | |
| 6 | (1S,2R)-2-(((S)-(5-cyclopropyl-6-fluoropyridin-2- | 382.2 | 383.1 |
| | yl)(phenyl)methyl)carbamoyl)cyclopentane-1-carboxylic acid | | |
| 7 | (1S,2R)-2-(((R)-(3-fluoro-4-isopropylphenyl)(1-methyl-2-oxo- | 453.2 | 454.1 |
| | 2,3-dihydro-1H-benzo[d]imidazol-4- | | |
| | yl)methyl)carbamoyl)cyclopentane-1-carboxylic acid | | |
| 8 | (1S,2R)-2-(((S)-(3,5-difluoro-4- | 401.2 | 402.2 |
| | isopropylphenyl)(phenyl)methyl)carbamoyl)cyclopentane-1- | | |
| | carboxylic acid | | |
| 9 | (1S,2R)-2-(((R)-(4-isopropylphenyl)(2-oxo-2,3- | 422.2 | 423.1 |
| | dihydrobenzo[d]oxazol-7-yl)methyl)carbamoyl)cyclopentane- | | |
| | 1-carboxylic acid | | |
| 10 | (1S,2R)-2-(((R)-(4-isopropylphenyl)(2-oxo-2,3- | 422.2 | 423.2 |
| | dihydrobenzo[d]oxazol-4-yl)methyl)carbamoyl)cyclopentane- | | |
| | 1-carboxylic acid | | |
| 11 | (1S or 1R,2R or 2S,4R or 4S)-4-((tert-butoxycarbonyl)amino)- | 480.3 | 481.5 |
| | 2-(((S)-(4- | | |
| | isopropylphenyl)(phenyl)methyl)carbamoyl)cyclopentane-1- | | |
| | carboxylic acid | | |
| 12 | (1S,2R)-2-(((S)-(5-cyclopropylpyridin-2- | 364.2 | 365.2 |
| | yl)(phenyl)methyl)carbamoyl)cyclopentane-1-carboxylic acid | | |
| 13 | (1S,2R)-2-(((R)-(2-(azetidine-3-carboxamido)phenyl)(4- | 463.2 | 464.1 |
| | isopropylphenyl)methyl)carbamoyl)cyclopentane-1-carboxylic | | |
| | acid | | |
| 14 | (1S,2R)-2-(((S)-(4-(1- | 377.2 | 378.2 |
| | methylcyclopropyl)phenyl)(phenyl)methyl)carbamoyl)cyclopentane- | | |
| | 1-carboxylic acid | | |
| 15 | (1S,2R)-2-(((S)-(5-cyclobutylpyridin-2- | 378.2 | 379.2 |
| | yl)(phenyl)methyl)carbamoyl)cyclopentane-1-carboxylic acid | | |
| 16 | (1S or 1R,2R or 2S,4R or 4S)-4-hydroxy-2-(((S)-(4- | 381.2 | 382.4 |
| | isopropylphenyl)(phenyl)methyl)carbamoyl)cyclopentane-1- | | |
| | carboxylic acid | | |
| 17 | (1S,2R)-2-(((S)-(4-cyclopropyl-3- | 381.2 | 382.1 |
| | fluorophenyl)(phenyl)methyl)carbamoyl)cyclopentane-1- | | |
| | carboxylic acid | | |
| 18 | (1S,2R)-2-(((S)-(4-cyclobutyl-3- | 395.2 | 396.2 |
| | fluorophenyl)(phenyl)methyl)carbamoyl)cyclopentane-1- | | |
| | carboxylic acid | | |
| 19 | (1S,2R)-2-(((R)-(4-isopropylphenyl)(2-oxo-2,3-dihydro-1H- | 421.2 | 422.1 |
| | benzo[d]imidazol-4-yl)methyl)carbamoyl)cyclopentane-1- | | |
| | carboxylic acid | | |
| 20 | (1S,2R)-2-(((R)-(4-isopropylphenyl)(2-oxo-2,3-dihydro-1H- | 421.2 | 422.1 |
| | benzo[d]imidazol-4-yl)methyl)carbamoyl)cyclopentane-1- | | |
| | carboxylic acid | | |
| 21 | (1S or 1R,2R or 2S,4S or 4R)-4-hydroxy-2-(((S)-(4- | 381.2 | 382.4 |
| | isopropylphenyl)(phenyl)methyl)carbamoyl)cyclopentane-1- | | |
| | carboxylic acid | | |
| 22 | (1S,2R)-2-(((S)-(5-isopropylpyridin-2-yl)(o- | 380.2 | 381.2 |
| | tolyl)methyl)carbamoyl)cyclopentane-1-carboxylic acid | | |
| 23 | (1S,2R)-2-(((R)-(3-fluoro-4-isopropylphenyl)(o- | 397.2 | 398.2 |
| | tolyl)methyl)carbamoyl)cyclopentane-1-carboxylic acid | | |
| 24 | (1S,2R)-2-(((R)-(3-fluoro-4-isopropylphenyl)(2- | 441.2 | 442.1 |
| | ureidophenyl)methyl)carbamoyl)cyclopentane-1-carboxylic | | |
| | acid | | |
| 25 | (1S or 1R,2R or 2S,4R or 4S)-4-fluoro-2-(((S)-(4- | 383.2 | 382.2 |
| | isopropylphenyl)(phenyl)methyl)carbamoyl)cyclopentane-1- | | (M − H) |
| | carboxylic acid | | |
| 26 | (1S,2R)-2-(((R)-(4-cyclopropylphenyl)(o- | 377.2 | 378.1 |
| | tolyl)methyl)carbamoyl)cyclopentane-1-carboxylic acid | | |
| 27 | (1S,2R)-2-(((R)-(3-fluoro-4-isopropylphenyl)(1H-pyrazol-5- | 373.2 | 374.2 |
| | yl)methyl)carbamoyl)cyclopentane-1-carboxylic acid | | |
| 28 | (1S,2R)-2-(((R)-(4-cyclobutylphenyl)(o- | 391.2 | 392.2 |
| | tolyl)methyl)carbamoyl)cyclopentane-1-carboxylic acid | | |
| 29 | (1S,2R)-2-(((R)-(2-aminopyridin-3-yl)(4- | 381.2 | 382.1 |
| | isopropylphenyl)methyl)carbamoyl)cyclopentane-1-carboxylic | | |
| | acid | | |
| 30 | (1S,2R)-2-(((R)-(4-isopropylphenyl)(2-oxoindolin-7- | 420.2 | 421.1 |
| | yl)methyl)carbamoyl)cyclopentane-1-carboxylic acid | | |
| 31 | (1S,2R)-2-(((R)-(4-isopropylphenyl)(2-oxo-1,2,3,4- | 434.2 | 435.2 |
| | tetrahydroquinolin-8-yl)methyl)carbamoyl)cyclopentane-1- | | |
| | carboxylic acid | | |
| 32 | (1S,2R)-2-(((R)-(4-isopropylphenyl)(1H-pyrazol-5- | 355.2 | 356.2 |
| | yl)methyl)carbamoyl)cyclopentane-1-carboxylic acid | | |
| 33 | (1S or 1R,3R or 3S)-3-(((S)-(4- | 365.2 | 366.2 |
| | isopropylphenyl)(phenyl)methyl)carbamoyl)cyclopentane-1- | | |
| | carboxylic acid | | |
| 34 | (1S,2R)-2-(((S)-(4- | 377.2 | 378.2 |
| | cyclobutylphenyl)(phenyl)methyl)carbamoyl)cyclopentane-1- | | |
| | carboxylic acid | | |
| 35 | (1S or 1R,2R or 2S)-2-(((R)-(6,6-dimethylspiro[3.3]heptan-2- | 369.2 | 370.2 |
| | yl)(phenyl)methyl)carbamoyl)cyclopentane-1-carboxylic acid | | |
| 36 | (1S,2R)-2-(((S)-(5-isopropylpyridin-2- | 366.2 | 367.1 |
| | yl)(phenyl)methyl)carbamoyl)cyclopentane-1-carboxylic acid | | |
| 37 | (1S,2R)-2-(((S)-(3-fluoro-4- | 383.2 | 384.2 |
| | isopropylphenyl)(phenyl)methyl)carbamoyl)cyclopentane-1- | | |
| | carboxylic acid | | |
| 38 | (1S,2R)-2-(((S)-(4- | 363.2 | 364.2 |
| | cyclopropylphenyl)(phenyl)methyl)carbamoyl)cyclopentane- | | |
| | 1-carboxylic acid | | |
| 39 | (1S,2R)-2-(((R)-(4-isopropylphenyl)(2- | 395.2 | 396.3 |
| | methoxyphenyl)methyl)carbamoyl)cyclopentane-1-carboxylic | | |
| | acid | | |
| 40 | (1S,2R)-2-(((R)-(4-isopropylphenyl)(o- | 379.2 | 380.3 |
| | tolyl)methyl)carbamoyl)cyclopentane-1-carboxylic acid | | |
| 41 | (1S,2R)-2-(((S)-(4- | 365.2 | 366.2 |
| | isopropylphenyl)(phenyl)methyl)carbamoyl)cyclopentane-1- | | |
| | carboxylic acid | | |
| 42 | (1S,2R)-2-(((R)-(4-isopropylphenyl)(thiophen-2- | 385.2 | 386.1 |
| | yl)methyl)carbamoyl)cyclohexane-1-carboxylic acid | | |
| 43 | (1R or 1S,2S or 2R)-2-(((S or R)-(4- | 371.2 | 372.2 |
| | isopropylphenyl)(thiophen-2- | | |
| | yl)methyl)carbamoyl)cyclopentane-1-carboxylic acid | | |
| 44 | (1R or 1S,2S or 2R)-2-(((R or S)-(4- | 371.2 | 372.2 |
| | isopropylphenyl)(thiophen-2- | | |
| | yl)methyl)carbamoyl)cyclopentane-1-carboxylic acid | | |
| 45 | (1S,2R)-2-(((R)-(4-isopropylphenyl)(thiophen-2- | 371.2 | 372.2 |
| | yl)methyl)carbamoyl)cyclopentane-1-carboxylic acid | | |
| 46 | (1S or 1R,2R or 2S)-2-(((S or R)-(4- | 371.2 | 372.2 |
| | isopropylphenyl)(thiophen-2- | | |
| | yl)methyl)carbamoyl)cyclopentane-1-carboxylic acid | | |

### Example S-2: Synthesis of (1S,2R)-2-(((R)-(3-fluoro-4-isopropylphenyl)(1-methyl-2-oxo-2,3-dihydro-1H-benzo[d]imidazol-4-yl)methyl) carbamoyl)cyclopentane-1-carboxylic acid (Compound 7)

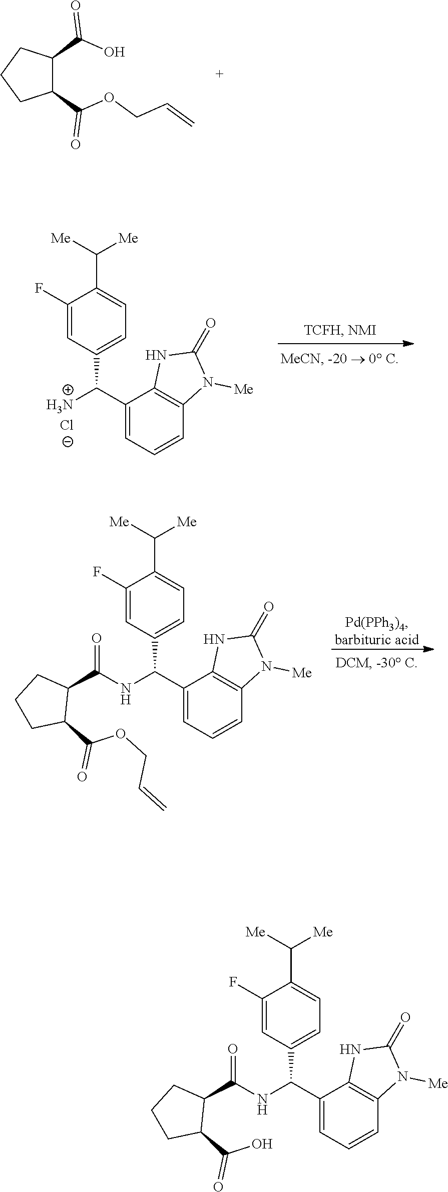

Step a: A mixture of (R)-(3-fluoro-4-isopropylphenyl)(1-methyl-2-oxo-2,3-dihydro-1H-benzo[d]imidazol-4-yl) methanaminium chloride (80.0 mg, 255 μmol, 1.00 eq) and (1R,2S)-2-((allyloxy) carbonyl)cyclopentane-1-carboxylic acid (60.7 mg, 306 μmol, 1.20 eq), N-methylimidazole (62.9 mg, 766 μmol, 3.00 eq) in CH₃CN (5.00 mL) at −20° C. was added chloro-N,N,N′,N′-tetramethylformamidinium hexafluorophosphate (85.9 mg, 306 μmol, 1.20 eq), and the resulting mixture was warmed to 0° C. and stirred for 1 h. The reaction mixture was then quenched by addition of H₂O (5 mL) at 0° C., and the resulting biphasic mixture was extracted with DCM (3×10 mL). The combined organic extracts were washed with brine (3×50 mL), dried over anhydrous Na₂SO₄, filtered, and concentrated under reduced pressure. The crude residue obtained was purified by column chromatography to give allyl(1S,2R)-2-(((R)-(3-fluoro-4-isopropylphenyl)(1-methyl-2-oxo-2,3-dihydro-1H-benzo[d]imidazol-4-yl)methyl) carbamoyl)cyclopentane-1-carboxylate. LC-MS (ESI): m/z: [M+H]⁺ calculated for C₂₈H₃₂FN₃O₄: 494.2. found 494.3.

Step b: To a solution of (1S,2R)-2-(((R)-(3-fluoro-4-isopropylphenyl)(1-methyl-2-oxo-2,3-dihydro-1H-benzo[d]imidazol-4-yl)methyl) carbamoyl)cyclopentane-1-carboxylate (130 mg, 263 μmol, 1.00 eq) in DCM (5.00 mL) at −30° C. under N₂ was added Pd(PPh₃)₄ (91.3 mg, 79.0 μmol, 0.3 eq) and barbituric acid (202 mg, 1.58 mmol, 6.00 eq). The resulting mixture was stirred at −30° C. for 1 h under N₂ atmosphere. The reaction mixture was then warmed to 0° C. and quenched by addition of H₂O (5.0 mL), and the resulting biphasic mixture was extracted with DCM (3×10 mL). The combined organic extracts were washed with brine (10 mL), dried over anhydrous Na₂SO₄, filtered, and concentrated under reduced pressure. The crude residue obtained was purified by prep-HPLC to give (1S,2R)-2-(((R)-(3-fluoro-4-isopropylphenyl)(1-methyl-2-oxo-2,3-dihydro-1H-benzo[d]imidazol-4-yl)methyl) carbamoyl)cyclopentane-1-carboxylic acid. LC-MS (ESI): m/z: [M+H]⁺ calculated for C₂₅H₂₈FN₃O₄: 454.2. found 454.2.

### Example S-3: Synthesis of (1S,2R)-2-(((R)-(3-fluoro-4-isopropylphenyl)(o-tolyl)methyl)carbamoyl)cyclopentane-1-carboxylic acid (Compound 23)

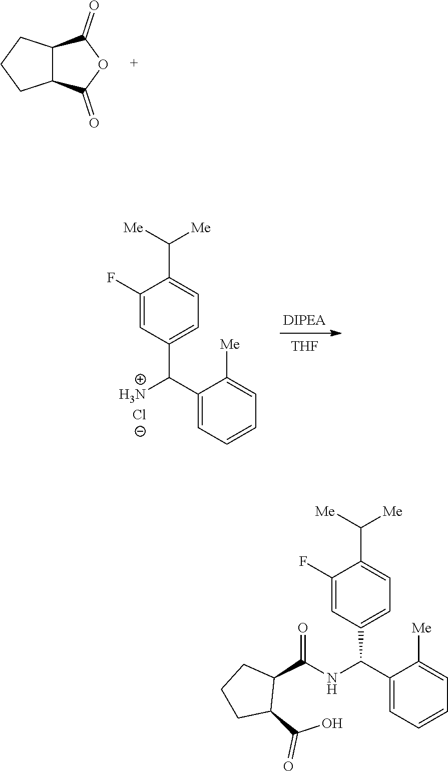

Step a: To a solution of (3-fluoro-4-isopropylphenyl)(o-tolyl) methanaminium chloride (500 mg, 1.70 mmol, 1 eq) and cis-tetrahydro-1H-cyclopenta[c]furan-1,3(3aH)-dione (262 mg, 1.87 mmol, 1.1 eq) in THF (4 mL) was added DIPEA (659 mg, 5.11 mmol, 3 eq). The resulting mixture was stirred at 20° C. for 2 h. The reaction mixture was then diluted with H₂O (10 mL) and extracted with EtOAc (2×20 mL). The organic extracts were dried over anhydrous Na₂SO₄, filtrated, and concentrated under reduced pressure. The crude residue obtained was purified by prep-HPLC to give a mixture of diastereomers. This mixture was separated using chiral SFC (column: DAICEL CHIRALPAK AD) to give (1S,2R)-2-(((R)-(3-fluoro-4-isopropylphenyl)(o-tolyl)methyl) carbamoyl)cyclopentane-1-carboxylic acid as the second eluting isomer. LC-MS (ESI): m/z: [M+H]⁺ calculated for C₂₄H₂₈FNO₃: 398.2. found 398.2.

The following compounds in Table T-1 were synthesized using procedures similar to Examples S-2 and S-3 using the appropriate starting materials.

TABLE T-1

| | | | Exact | LCMS, |
|---|---|---|---|---|
| Cmpd. | | | mass | Found |
| No. | Structure | IUPAC | (g/mol) | [M + H]⁺ |
| 1 | 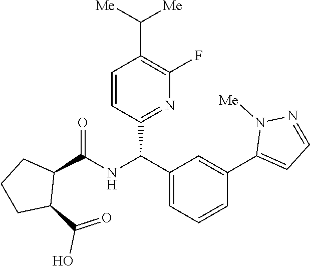 [SMILES: CC(C)c1ccc([C@@H](NC(=O)[C@@H]2CCC[C@@H]2C(=O)O)c2cccc(-c3ccnn3C)c2)nc1F] | (1S,2R)-2-(((S)-(6- fluoro-5- isopropylpyridin-2- yl)(3-(1-methyl-1H- pyrazol-5- yl)phenyl)methyl) carbamoyl)cyclopentane- 1-carboxylic acid | 464.2 | 465.1 |
| 2 | 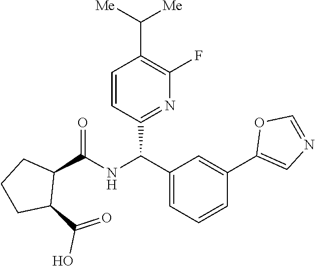 [SMILES: CC(C)c1ccc([C@@H](NC(=O)[C@@H]2CCC[C@@H]2C(=O)O)c2cccc(-c3cnco3)c2)nc1F] | (1S,2R)-2-(((S)-(6- fluoro-5- isopropylpyridin-2- yl)(3-(oxazol-5- yl)phenyl)methyl) carbamoyl)cyclopentane- 1-carboxylic acid | 451.2 | 452.1 |
| 3 | 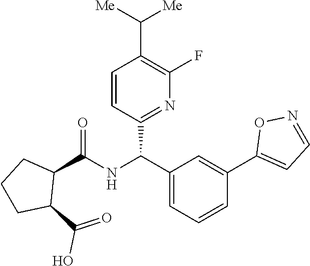 [SMILES: CC(C)c1ccc([C@@H](NC(=O)[C@@H]2CCC[C@@H]2C(=O)O)c2cccc(-c3ccno3)c2)nc1F] | (1S,2R)-2-(((S)-(6- fluoro-5- isopropylpyridin-2- yl)(3-isoxazol-5- yl)phenyl)methyl) carbamoyl)cyclopentane- 1-carboxylic acid | 451.2 | 452.2 |
| 4 | 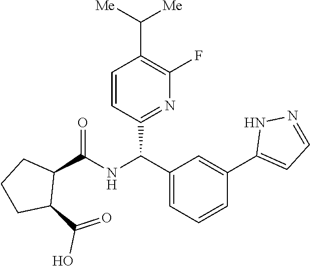 [SMILES: CC(C)c1ccc([C@@H](NC(=O)[C@@H]2CCC[C@@H]2C(=O)O)c2cccc(-c3ccn[nH]3)c2)nc1F] | (1S,2R)-2-(((S)-(3-(1H- pyrazol-5-yl)phenyl)(6- fluoro-5- isopropylpyridin-2- yl)methyl)carbamoyl) cyclopentane-1- carboxylic acid | 450.2 | 451.1 |
| 5 | 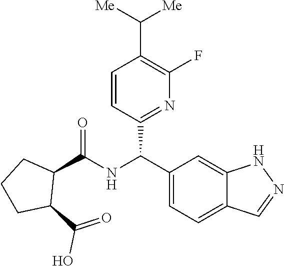 [SMILES: CC(C)c1ccc([C@@H](NC(=O)[C@@H]2CCC[C@@H]2C(=O)O)c2ccc3cn[nH]c3c2)nc1F] | (1S,2R)-2-(((S)-(6- fluoro-5- isopropylpyridin-2- yl)(1H-indazol-6- yl)methyl)carbamoyl) cyclopentane-1- carboxylic acid | 424.2 | 425.1 |
| 6 | 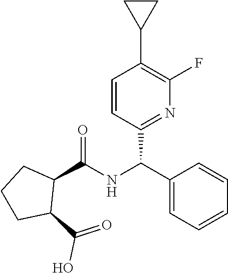 [SMILES: O=C(O)[C@H]1CCC[C@H]1C(=O)N[C@@H](c1ccccc1)c1ccc(C2CC2)c(F)n1] | (1S,2R)-2-(((S)-(5- cyclopropyl-6- fluoropyridin-2- yl)(phenyl)methyl) carbamoyl)cyclopentane- 1-carboxylic acid | 382.2 | 383.1 |
| 8 | 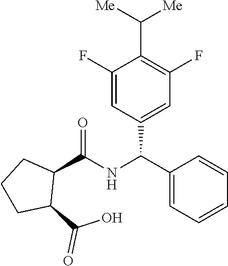 [SMILES: CC(C)c1c(F)cc([C@@H](NC(=O)[C@@H]2CCC[C@@H]2C(=O)O)c2ccccc2)cc1F] | (1S,2R)-2-(((S)-(3,5- difluoro-4- isopropylphenyl) (phenyl)methyl) carbamoyl)cyclopentane- 1-carboxylic acid | 401.2 | 402.2 |
| 9 | 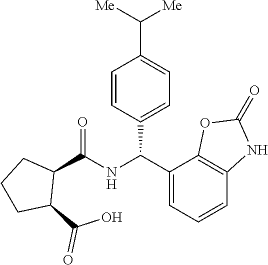 [SMILES: CC(C)c1ccc([C@@H](NC(=O)[C@@H]2CCC[C@@H]2C(=O)O)c2cccc3[nH]c(=O)oc23)cc1] | (1S,2R)-2-(((R)-(4- isopropylphenyl)(2-oxo- 2,3-dihydrobenzo[d] oxazol-7-yl)methyl) carbamoyl)cyclopentane- carboxylic acid | 422.2 | 423.1 |
| 10 | 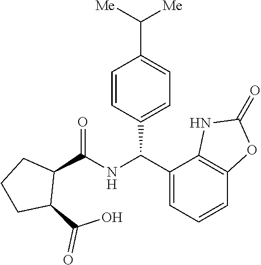 [SMILES: CC(C)c1ccc([C@@H](NC(=O)[C@@H]2CCC[C@@H]2C(=O)O)c2cccc3oc(=O)[nH]c23)cc1] | (1S,2R)-2-(((R)-(4- isopropylphenyl)(2-oxo- 2,3-dihydrobenzo[d] oxazol-4-yl)methyl) carbamoyl)cyclopentane- 1-carboxylic acid, SFC column: DAICEL CHIRALPAK AD, first eluting isomer | 422.2 | 423.2 |
| 11 | 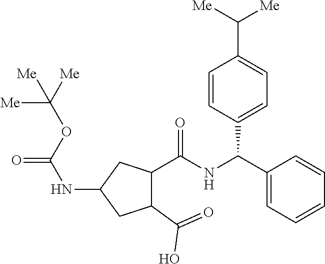 [SMILES: CC(C)c1ccc([C@@H](NC(=O)C2CC(NC(=O)OC(C)(C)C)CC2C(=O)O)c2ccccc2)cc1] | (1S or 1R,2R or 2S,4R or 4S)-4-((tert- butoxycarbonyl)amino)- 2-(((S)-4- isopropylphenyl)(phenyl) methyl)carbamoyl) cyclopentane-1-carboxylic acid | 480.3 | 481.5 |
| 12 | 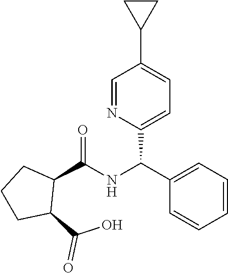 [SMILES: O=C(O)[C@H]1CCC[C@H]1C(=O)N[C@@H](c1ccccc1)c1ccc(C2CC2)cn1] | (1S,2R)-2-(((S)-(5- cyclopropylpyridin-2- yl)(phenyl)methyl) carbamoyl)cyclopentane-1- carboxylic acid, SFC column: DAICEL CHIRALPAK AD, second eluting isomer | 364.2 | 365.2 |
| 13 | 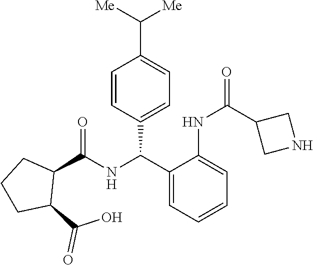 [SMILES: CC(C)c1ccc([C@@H](NC(=O)[C@@H]2CCC[C@@H]2C(=O)O)c2ccccc2NC(=O)C2CNC2)cc1] | (1S,2R)-2-(((R)-(2- (azetidine-3- carboxamido)phenyl)(4- isopropylphenyl)methyl) carbamoyl)cyclopentane- 1-carboxylic acid | 463.2 | 464.1 |
| 14 | 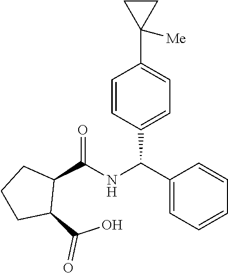 [SMILES: CC1(c2ccc([C@@H](NC(=O)[C@@H]3CCC[C@@H]3C(=O)O)c3ccccc3)cc2)CC1] | (1S,2R)-2-(((S)-(4-(1- methylcyclopropyl) phenyl)(phenyl)methyl) carbamoyl)cyclopentane-1- carboxylic acid, SFC column: DAICEL CHIRALPAK AD, second eluting isomer | 377.2 | 378.2 |
| 15 | 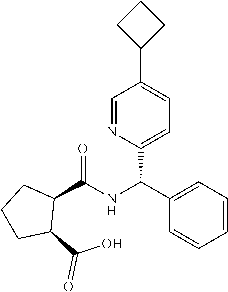 [SMILES: O=C(O)[C@H]1CCC[C@H]1C(=O)N[C@@H](c1ccccc1)c1ccc(C2CCC2)cn1] | (1S,2R)-2-(((S)-(5- cyclobutylpyridin-2- yl)(phenyl)methyl) carbamoyl)cyclopentane-1- carboxylic acid, SFC column: DAICEL CHIRALPAK AD, fourth eluting isomer | 378.2 | 379.2 |
| 16 | 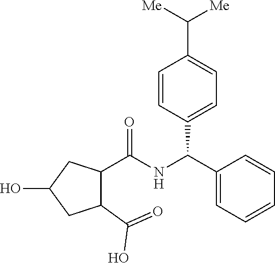 [SMILES: CC(C)c1ccc([C@@H](NC(=O)C2CC(O)CC2C(=O)O)c2ccccc2)cc1] | (1S or 1R,2R or 2S,4R or 4S)-4-hydroxy-2- (((S)-(4- isopropylphenyl)(phenyl) methyl)carbamoyl) cyclopentane-1-carboxylic acid | 381.2 | 382.4 |
| 17 | 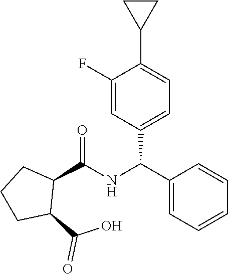 [SMILES: O=C(O)[C@H]1CCC[C@H]1C(=O)N[C@@H](c1ccccc1)c1ccc(C2CC2)c(F)c1] | (1S,2R)-2-(((S)-(4- cyclopropyl-3- fluorophenyl)(phenyl) methyl)carbamoyl) cyclopentane-1-carboxylic acid, SFC column: DAICEL CHIRALPAK AD, second eluting isomer | 381.2 | 382.1 |
| 18 | 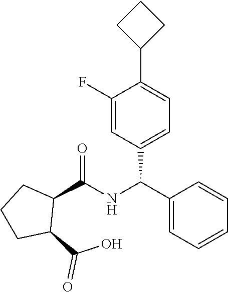 [SMILES: O=C(O)[C@H]1CCC[C@H]1C(=O)N[C@@H](c1ccccc1)c1ccc(C2CCC2)c(F)c1] | (1S,2R)-2-(((S)-(4- cyclobutyl-3- fluorophenyl)(phenyl) methyl)carbamoyl) cyclopentane-1-carboxylic acid, SFC column: DAICEL CHIRALPAK AD, second eluting isomer | 395.2 | 396.2 |
| 19 | 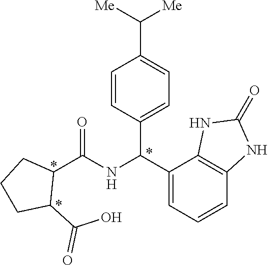 [SMILES: CC(C)c1ccc(C(NC(=O)C2CCCC2C(=O)O)c2cccc3[nH]c(=O)[nH]c23)cc1] | (1S or 1R,2R or 2S)-2- (((R or S)-(4- isopropylphenyl)(2-oxo- 2,3-dihydro-1H- benzo[d]imidazol-4- yl)methyl)carbamoyl) cyclopentane-1-carboxylic acid, SFC column: DAICEL CHIRALPAK AD, first eluting isomer | 421.2 | 422.1 |
| 20 | 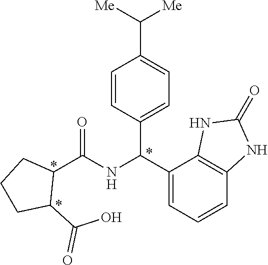 [SMILES: CC(C)c1ccc(C(NC(=O)C2CCCC2C(=O)O)c2cccc3[nH]c(=O)[nH]c23)cc1] | (1R or 1S,2S or 2R)-2- (((S or R)-(4- isopropylphenyl)(2-oxo- 2,3-dihydro-1H- benzo[d]imidazol-4- yl)methyl)carbamoyl) cyclopentane-1-carboxylic acid, SFC column: DAICEL CHIRALPAK AD, second eluting isomer | 421.2 | 422.1 |
| 21 | 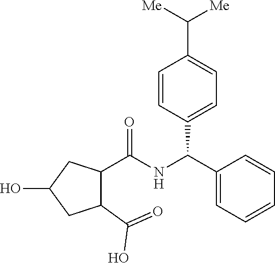 [SMILES: CC(C)c1ccc([C@@H](NC(=O)C2CC(O)CC2C(=O)O)c2ccccc2)cc1] | (1S or 1R,2R or 2S,4S or 4R)-4-hydroxy-2-(((S)- (4- isopropylphenyl)(phenyl) methyl)carbamoyl) cyclopentane-1-carboxylic acid | 381.2 | 382.4 |
| 22 | 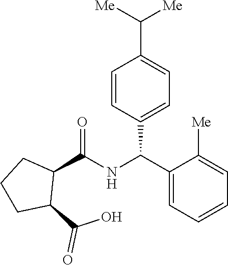 [SMILES: Cc1ccccc1[C@H](NC(=O)[C@@H]1CCC[C@@H]1C(=O)O)c1ccc(C(C)C)cc1] | (1S,2R)-2-(((S)-(5- isopropylpyridin-2- yl)(o- tolyl)methyl)carbamoyl) cyclopentane-1- carboxylic acid, SFC column: REGIS (s,s) WHELK-O1, first eluting isomer | 380.2 | 381.2 |
| 24 | 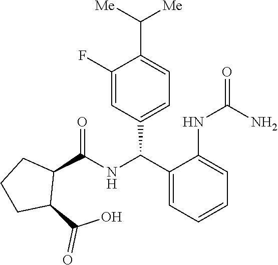 [SMILES: CC(C)c1ccc([C@@H](NC(=O)[C@@H]2CCC[C@@H]2C(=O)O)c2ccccc2NC(N)=O)cc1F] | (1S,2R)-2-(((R)-(3- fluoro-4- isopropylphenyl)(2- ureidophenyl)methyl) carbamoyl)cyclopentane-1- carboxylic acid, SFC column: DAICEL CHIRALPAK IC, second eluting isomer | 441.2 | 442.1 |
| 25 | 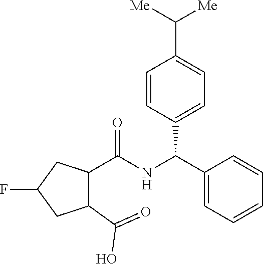 [SMILES: CC(C)c1ccc([C@@H](NC(=O)C2CC(F)CC2C(=O)O)c2ccccc2)cc1] | (1S or 1R,2R or 2S,4R or 4S)-4-fluoro-2-(((S)- (4-isopropylphenyl)(phenyl) methyl)carbamoyl) cyclopentane-1-carboxylic acid | 383.2 | 382.2 (M − H) |
| 26 | 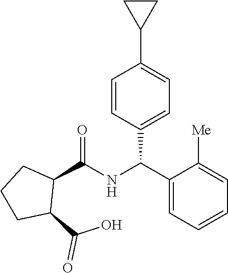 [SMILES: Cc1ccccc1[C@H](NC(=O)[C@@H]1CCC[C@@H]1C(=O)O)c1ccc(C2CC2)cc1] | (1S,2R)-2-(((R)-(4- cyclopropylphenyl)(o- tolyl)methyl)carbamoyl) cyclopentane-1- carboxylic acid, SFC column: DAICEL CHIRALPAK AD, second eluting isomer | 377.2 | 378.1 |
| 27 | 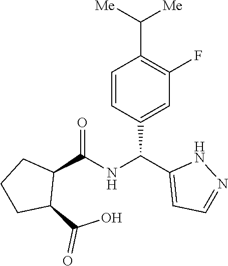 [SMILES: CC(C)c1ccc([C@@H](NC(=O)[C@@H]2CCC[C@@H]2C(=O)O)c2ccn[nH]2)cc1F] | (1S,2R)-2-(((R)-(3- fluoro-4- isopropylphenyl)(1H- pyrazol-5- yl)methyl)carbamoyl) cyclopentane-1-carboxylic acid, SFC SFC column: REGIS (s,s) WHELK- O1), second eluting isomer | 373.2 | 374.2 |
| 28 | 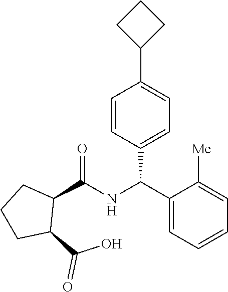 [SMILES: Cc1ccccc1[C@H](NC(=O)[C@@H]1CCC[C@@H]1C(=O)O)c1ccc(C2CCC2)cc1] | (1S,2R)-2-(((R)-(4- cyclobutylphenyl)(o- tolyl)methyl)carbamoyl) cyclopentane-1- carboxylic acid, SFC column: DAICEL CHIRALPAK AD, second eluting isomer | 391.2 | 392.2 |
| 29 | 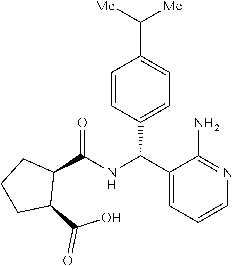 [SMILES: CC(C)c1ccc([C@@H](NC(=O)[C@@H]2CCC[C@@H]2C(=O)O)c2cccnc2N)cc1] | (1S,2R)-2-(((R)-(2- aminopyridin-3-yl)(4- isopropylphenyl)methyl) carbamoyl)cyclopentane-1- carboxylic acid, SFC column: DAICEL CHIRALPAK IC, second eluting isomer | 381.2 | 382.1 |
| 30 | 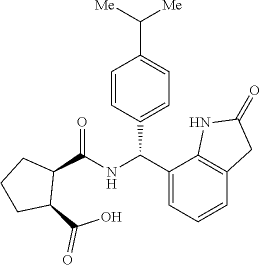 [SMILES: CC(C)c1ccc([C@@H](NC(=O)[C@@H]2CCC[C@@H]2C(=O)O)c2cccc3c2NC(=O)C3)cc1] | (1S,2R)-2-(((R)-(4- isopropylphenyl)(2- oxoindolin-7- yl)methyl)carbamoyl) cyclopentane-1-carboxylic acid, SFC column: DAICEL CHIRALPAK IC, second eluting isomer | 420.2 | 421.1 |
| 31 | 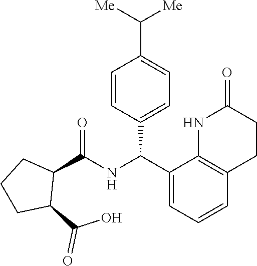 [SMILES: CC(C)c1ccc([C@@H](NC(=O)[C@@H]2CCC[C@@H]2C(=O)O)c2cccc3c2NC(=O)CC3)cc1] | (1S,2R)-2-(((R)-(4- isopropylphenyl)(2-oxo- 1,2,3,4- tetrahydroquinolin-8- yl)methyl)carbamoyl) cyclopentane-1-carboxylic acid, SFC column: DAICEL CHIRALPAK AD, second eluting isomer | 434.2 | 435.2 |
| 32 | 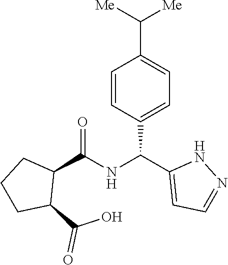 [SMILES: CC(C)c1ccc([C@@H](NC(=O)[C@@H]2CCC[C@@H]2C(=O)O)c2ccn[nH]2)cc1] | (1S,2R)-2-(((R)-(4- isopropylphenyl)(1H- pyrazol-5- yl)methyl)carbamoyl) cyclopentane-1-carboxylic acid, prep-HPLC column: Phenomenex Luna C18, second eluting isomer | 355.2 | 356.2 |
| 33 | 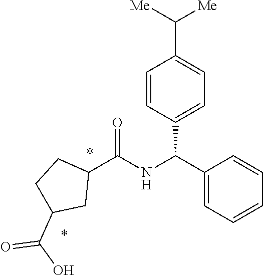 [SMILES: CC(C)c1ccc([C@@H](NC(=O)C2CCC(C(=O)O)C2)c2ccccc2)cc1] | (1S or 1R,3R or 3S)-3- (((S)-(4- isopropylphenyl)(phenyl) methyl)carbamoyl) cyclopentane-1-carboxylic acid, SFC column: Chiralcel OX-H, third eluting isomer | 365.2 | 366.2 |
| 34 | 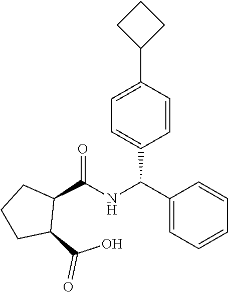 [SMILES: O=C(O)[C@H]1CCC[C@H]1C(=O)N[C@@H](c1ccccc1)c1ccc(C2CCC2)cc1] | (1S,2R)-2-(((S)-(4- cyclobutylphenyl)(phenyl) methyl)carbamoyl) cyclopentane-1-carboxylic acid, SFC column: DAICEL CHIRALPAK AD, second eluting isomer | 377.2 | 378.2 |
| 35 | 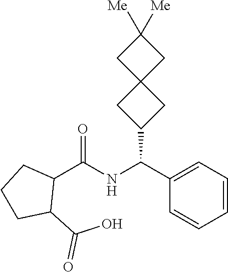 [SMILES: CC1(C)CC2(CC([C@@H](NC(=O)C3CCCC3C(=O)O)c3ccccc3)C2)C1] | (1S or 1R,2R or 2S)-2- (((R)-(6,6- dimethylspiro[3.3]heptan- 2-yl)(phenyl)methyl) carbamoyl)cyclopentane-1- carboxylic acid | 369.2 | 370.2 |
| 36 | 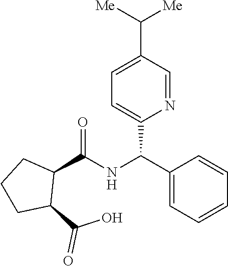 [SMILES: CC(C)c1ccc([C@@H](NC(=O)[C@@H]2CCC[C@@H]2C(=O)O)c2ccccc2)nc1] | (1S,2R)-2-(((S)-(5- isopropylpyridin-2- yl)(phenyl)methyl) carbamoyl)cyclopentane-1- carboxylic acid | 366.2 | 367.1 |
| 37 | 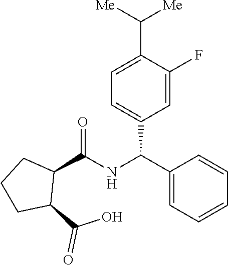 [SMILES: CC(C)c1ccc([C@@H](NC(=O)[C@@H]2CCC[C@@H]2C(=O)O)c2ccccc2)cc1F] | (1S,2R)-2-(((S)-(3- fluoro-4- isopropylphenyl)(phenyl) methyl)carbamoyl) cyclopentane-1-carboxylic acid, SFC column: DAICEL CHIRALPAK AD, first eluting isomer | 383.2 | 384.2 |
| 38 | 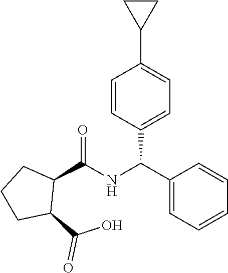 [SMILES: O=C(O)[C@H]1CCC[C@H]1C(=O)N[C@@H](c1ccccc1)c1ccc(C2CC2)cc1] | (1S,2R)-2-(((S)-(4- cyclopropylphenyl)(phenyl) methyl)carbamoyl) cyclopentane-1-carboxylic acid, SFC column: DAICEL CHIRALPAK AD, second eluting isomer | 363.2 | 364.2 |
| 39 | 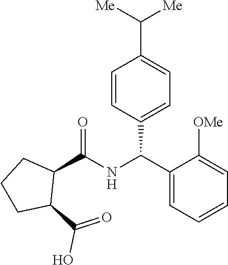 [SMILES: COc1ccccc1[C@H](NC(=O)[C@@H]1CCC[C@@H]1C(=O)O)c1ccc(C(C)C)cc1] | (1S,2R)-2-(((R)-(4- isopropylphenyl)(2- methoxyphenyl)methyl) carbamoyl)cyclopentane- 1-carboxylic acid | 395.2 | 396.3 |
| 40 | 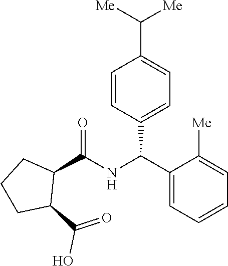 [SMILES: Cc1ccccc1[C@H](NC(=O)[C@@H]1CCC[C@@H]1C(=O)O)c1ccc(C(C)C)cc1] | (1S,2R)-2-(((R)-(4- isopropylphenyl)(o- tolyl)methyl)carbamoyl) cyclopentane-1- carboxylic acid, SFC column: DAICEL CHIRALPAK AD, second eluting isomer | 379.2 | 380.3 |
| 41 | 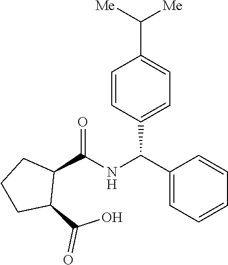 [SMILES: CC(C)c1ccc([C@@H](NC(=O)[C@@H]2CCC[C@@H]2C(=O)O)c2ccccc2)cc1] | (1S,2R)-2-(((S)-(4- isopropylphenyl)(phenyl) methyl)carbamoyl)cyclo- pentane-1-carboxylic acid | 365.2 | 366.2 |
| 42 | 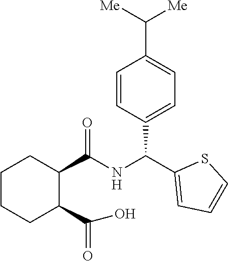 [SMILES: CC(C)c1ccc([C@@H](NC(=O)[C@@H]2CCCC[C@@H]2C(=O)O)c2cccs2)cc1] | (1S,2R)-2-(((R)-(4- isopropylphenyl)(thiophen- 2-yl)methyl)carbamoyl) cyclohexane-1-carboxylic acid, SFC column: (S,S) Whelk O-1, second eluting isomer | 385.2 | 386.1 |
| 43 | 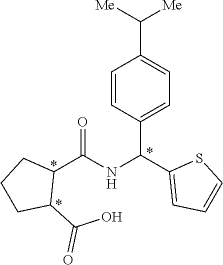 [SMILES: CC(C)c1ccc(C(NC(=O)C2CCCC2C(=O)O)c2cccs2)cc1] | (1R or 1S,2S or 2R)-2- (((S or R)-(4- isopropylphenyl)(thiophen- 2-yl)methyl)carbamoyl) cyclopentane-1-carboxylic acid, SFC column: Chiralpak AD-H, fourth eluting isomer | 371.2 | 372.2 |
| 44 | 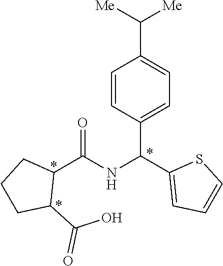 [SMILES: CC(C)c1ccc(C(NC(=O)C2CCCC2C(=O)O)c2cccs2)cc1] | (1R or 1S,2S or 2R)-2- (((R or S)-(4- isopropylphenyl)(thiophen- 2-yl)methyl)carbamoyl) cyclopentane-1-carboxylic acid, SFC column: Chiralpak AD-H, third eluting isomer | 371.2 | 372.2 |
| 45 | 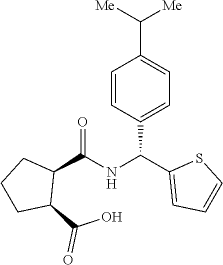 [SMILES: CC(C)c1ccc([C@@H](NC(=O)[C@@H]2CCC[C@@H]2C(=O)O)c2cccs2)cc1] | (1S,2R)-2-(((R)-(4- isopropylphenyl)(thiophen- 2-yl)methyl)carbamoyl) cyclopentane-1-carboxylic acid, SFC column: Chiralpak AD-H, second eluting isomer | 371.2 | 372.2 |
| 46 | 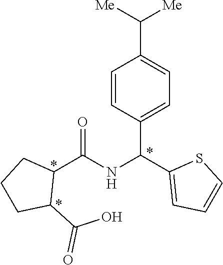 [SMILES: CC(C)c1ccc(C(NC(=O)C2CCCC2C(=O)O)c2cccs2)cc1] | (1S or 1R,2R or 2S)-2- (((S or R)-(4- isopropylphenyl)(thiophen- 2-yl)methyl)carbamoyl) cyclopentane-1-carboxylic acid, SFC column: Chiralpak AD-H, first eluting isomer | 371.2 | 372.2 |

### BIOLOGICAL EXAMPLES

### Example B-1

The GYS1 coupled enzyme assay is a kinetic biochemical assay that indirectly quantifies the rate of glycogen synthesis by coupling the conversion of GYS1 substrate UDP-glucose into UDP with downstream enzymatic reactions. UDP is released from UDP-glucose as glucose monomers are linked into the growing glycogen strand by GYS1. The coupled assay then proceeds with pyruvate kinase utilizing UDP and phospho (enol) pyruvate (PEP) to form pyruvate. Lactate dehydrogenase then converts pyruvate and NADH into lactate and NAD+. Oxidation of NADH to NAD+ can be measured continuously with a plate reader by quantifying the decrease in NADH absorbance at 340 nm over time.

Compounds that inhibit the hGYS1 enzyme and, subsequently, the downstream conversion of NADH to NAD+, were tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP at 1 mM were added before splitting buffer into hGYS1 buffer and substrate buffer. To the hGYS1 buffer, rabbit liver glycogen was added at a final concentration of 0.2% glycogen. Glucose-6-Phosphate was added at 1 mM, recombinant hGYS1/GN1 protein was added at 50 nM to the substrate buffer, phosphoenolpyruvate (PEP) was added at 2 mM, UDP-Glucose was added at 0.8 mM, NADH) was added at 0.6 mM, and Pyruvate Kinase/Lactate Dehydrogenase was added at 20 units/mL. The reaction was initiated by mixing hGYS1 buffer and substrate buffer at a 1:1 ratio. Both buffers were plated using a liquid dispensing device with hGYS1 buffer plated first followed by the substrate buffer. Plates were spun briefly to eliminate air bubbles and are immediately read in continuous mode at an absorbance of 340 nm, for 10 time points in one-minute increments, for a total of 10 minutes. The slope from these 10 time points was normalized to the positive and negative control wells. The duplicate % inhibition values are then averaged and fit to a Hill equation for dose response according to the Levenberg-Marquardt algorithm with the Hill equation maximum set to 100 and the minimum set to 0.

The results are shown in Table 3 below, which reports the IC₅₀ of each compound. Unless otherwise specified, IC₅₀ values are reported as the geometric mean of at least 2 assay runs on separate days. Each run represents the average of a technical replicate, where each compound was assayed twice in the same plate. As shown in the table below, the compounds of the present invention are potent inhibitors of human GYS1.

Note that, in Table 3, the compounds are referred to by the corresponding Compound No. in Table 1, which is also referred to in the synthetic examples.

TABLE 3

| | GYS1 |
|---|---|
| Cmpd | PK/LDH |
| No. | IC50 (μM) |
| 1 | 1.442 |
| 2 | 0.426 |
| 3 | 0.630 |
| 4 | 0.178 |
| 5 | 0.084 |
| 6 | 0.272 |
| 7 | 0.884 |
| 8 | 0.239 |
| 9 | 0.879 |
| 10 | 2.934 |
| 11 | 1.945 |
| 12 | 1.731 |
| 13 | 2.362 |
| 14 | 1.005 |
| 15 | 0.497 |
| 16 | 0.836 |
| 17 | 1.582 |
| 18 | 0.384 |
| 19 | 0.228 |
| 20 | 2.378 |
| 21 | 1.956 |
| 22 | 1.265 |
| 23 | 0.166 |
| 24 | 2.823 |
| 25 | 1.147 |
| 26 | 0.795 |
| 27 | 1.478 |
| 28 | 0.224 |
| 29 | 1.516 |
| 30 | 1.606 |
| 31 | 1.086 |
| 32 | 2.392 |
| 33 | 3.128 |
| 34 | 0.426 |
| 35 | 2.395 |
| 36 | 0.837 |
| 37 | 0.236 |
| 38 | 1.129 |
| 39 | 3.405 |
| 40 | 0.390 |
| 41 | 0.637 |
| 42 | 0.961 |
| 43 | 19.750 |
| 44 | 12.870 |
| 45 | 0.364 |
| 46 | >99.88 |

### Example B-2

The GYS1 cell based assay is a bioluminescent assay that quantifies the glucose resulting from glycogen digestion; the quantified glucose is an indirect measure of GYS1 glycogen synthesis. Newly synthesized glycogen is digested using Glucoamylase; the resulting glucose is quantified by using the Glucose-glo assay kit from Promega. Glucose-glo works by coupling glucose oxidation and NADH production with a bioluminescent system that is activated with NADH. Glucose is oxidized by Glucose dehydrogenase and the reaction reduces NAD+ to NADH; NADH activates Reductase which reduces a pro-luciferin Reductase Substrate to luciferin. Luciferin is detected in a luciferase reaction using Ultra-Glo rLuciferase and ATP, and the luminesce produced is proportional to the glucose in the sample. The luminescence is measured as a single point read in a plate reader.

Compounds that inhibit the hGYS1 enzyme and, subsequently, the glycogen synthesis in cells, were tested using assay ready plates (white, clear bottom 384 well plates) in a final DMSO reaction volume of 1% DMSO. Compounds in the assay ready plates were mixed with media with no additives, except for 20 mM glucose prior to cell addition. HeLa cells were starved in media with no additives, except for 1× Glutamax for 24 h. Starved HeLa cells were plated, in a 1:1 ratio to the media in the assay ready plate and incubated for 24 h at 37° C. and 5% CO₂. Cells were washed in 1×PBS buffer and lysed in lysis buffer containing 50% 1×PBS and 25% 0.3 N HCl of the final volume in the well or reaction volume; cells were incubated with lysis buffer for 10 minutes and quenched with the remaining 25% of the reaction volume that consisted of 450 mM Tris pH 8.0. Lysates were mixed in a 1:1 ratio with Glucoamylase in 100 mM Sodium Acetate buffer, pH 5.3; the mixture was incubated for 1 h at 37° C. The digested lysate was mixed in a 1:1 ratio with Glucose-glo detection mixture as per vendor recommendations (Luciferase detection buffer, Reductase, Reductase substrate, Glucose dehydrogenase, and NAD) in read-out plates (solid white 384-well plates) and incubated for 1 h at RT. The plates were read using a plate reader with luminescence capabilities. Each compound concentration Relative Luminescence Unit (RLU) was averaged and normalized to the average RLU of the positive and negative controls to obtain a percentage inhibition. The normalized data vs. concentration was plotted; to determine the half-maximal concentration (IC₅₀), the Levenberg-Marquardt algorithm was used to fit a Hill equation to the dose response data.

The results are shown in Table 4 below, which reports the IC₅₀ of each compound. Unless otherwise specified, IC₅₀ values are reported as the geometric mean of at least 2 assay runs on separate days. As shown in the table below, the compounds of the present invention are potent inhibitors of human GYS1. Unless otherwise specified, IC₅₀ values are reported as the geometric mean of at least two assay runs on separate days. Each run represents the average of a technical replicate, where each compound was assayed twice in the same plate.

TABLE 4

| | Cellular |
|---|---|
| Cmpd No. | IC₅₀ (μM) |
| 7 | >10.00 |
| 8 | 3.93 |
| 9 | >100.00 |
| 10 | >100.00 |
| 11 | >100.00 |
| 12 | >65.78 |
| 13 | >68.62 |
| 14 | >60.78 |
| 15 | 8.49 |
| 16 | >100.00 |
| 17 | 27.3 |
| 18 | 16.57 |
| 19 | 20.68 |
| 20 | >100.00 |
| 21 | >100.00 |
| 22 | 13.9 |
| 23 | 5.36 |
| 24 | 91.97 |
| 25 | 24.48 |
| 26 | 21.57 |
| 27 | 46.29 |
| 28 | 15.77 |
| 29 | 65.58 |
| 30 | 31.32 |
| 31 | 12.26 |
| 32 | >100.00 |
| 37 | 3.69 |
| 39 | 41.28 |
| 45 | 6.6 |

### Example B-3

The GYS2 coupled enzyme assay is a kinetic biochemical assay that indirectly quantifies the rate of glycogen synthesis by coupling the conversion of GYS2 substrate UDP-glucose into UDP with downstream enzymatic reactions. UDP is released from UDP-glucose as glucose monomers are linked into the growing glycogen strand by GYS2. The coupled assay then proceeds with pyruvate kinase utilizing UDP and phospho (enol) pyruvate (PEP) to form pyruvate. Lactate dehydrogenase then converts pyruvate and NADH into lactate and NAD+. Oxidation of NADH to NAD+ can be measured continuously with a plate reader by quantifying the decrease in NADH absorbance at 340 nm over time.

Compounds that inhibit the hGYS2 enzyme and, subsequently, the downstream conversion of NADH to NAD+, were tested using assay ready plates (black, clear bottom 384 well plates) in a final DMSO reaction volume of 2.5% DMSO. The Assay Buffer contained 50 mM Tris pH 7.5, 2 mM MgCl₂, and 100 mM KCl. Fresh stocks of BSA at a final concentration of 0.02% and TCEP 1 mM were added before splitting buffer into hGYS2 buffer and substrate buffer. To the hGYS2 buffer, rabbit liver glycogen was added at a final concentration of 0.2% glycogen. Glucose-6-Phosphate was added at 2 mM, recombinant hGYS2/GN1 protein was added at 200 nM to the substrate buffer, phosphoenolpyruvate (PEP) was added at 2 mM, UDP-Glucose was added at 2 mM, NADH was added at 0.6 mM, and Pyruvate Kinase/Lactate Dehydrogenase was added at 20 units/mL. The reaction was initiated by mixing hGYS2 buffer and substrate buffer at a 1:1 ratio. Both buffers were plated using a liquid dispensing device with hGYS2 buffer plated first followed by the substrate buffer. Plates were spun briefly to eliminate air bubbles and are immediately read in continuous mode at an absorbance of 340 nm, for 10 time points in one-minute increments, for a total of 10 minutes. The slope from these 10 time points was normalized to the positive and negative control wells. The duplicate % inhibition values are then averaged and fit to a Hill equation for dose response according to the Levenberg-Marquardt algorithm with the Hill equation maximum set to 100 and the minimum set to 0.

The results are shown in Table 5 below, which reports the IC₅₀ of each compound. Unless otherwise specified, IC₅₀ values are reported as the geometric mean of at least 2 assay runs on separate days. As shown in the table below, the compounds of the present invention are not potent inhibitors of human GYS2. Unless otherwise specified, IC₅₀ values are reported as the geometric mean of at least two assay runs on separate days. Each run represents the average of a technical replicate, where each compound was assayed twice in the same plate.

TABLE 5

| | GYS2 |
|---|---|
| | PK/LDH |
| Cmpd No. | IC₅₀ (μM) |
| 8 | >100.0 |
| 15 | >100.0 |
| 17 | >100.0 |
| 18 | 28.6 |
| 19 | >100.0 |
| 22 | 66.73 |
| 23 | 8.483 |
| 26 | 15.49 |
| 27 | >100.0 |
| 28 | 7.797 |
| 31 | >100.0 |
| 34 | 67.07 |
| 36 | >100.0 |
| 37 | 72.75 |
| 39 | 66.4 |
| 40 | 10.78 |
| 41 | >87.06 |
| 42 | >77.60 |
| 43 | >250.0 |
| 44 | 11.69 |
| 45 | 97.96 |
| 46 | >250.0 |

### Example B-4

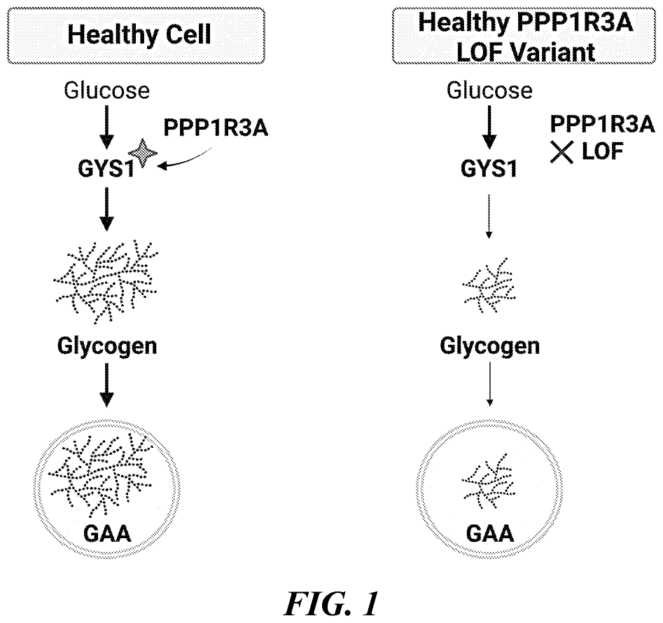

Pompe disease is a glycogen storage disease caused by mutations in the enzyme acid alpha-glucosidase resulting in pathological accumulation of glycogen. Glycogen can accumulate in virtually all tissues, but the primary pathology affects skeletal and cardiac muscle. Inhibiting the synthesis of muscle glycogen could reduce the pathologic build-up of glycogen by acting as a substrate reduction therapy. Savage et. al. identified a predicted protein truncating variant (PTV) in the PPP1R3A gene (a regulator of glycogen metabolism) in ˜0.5% of Europeans, which results in ˜65% reduction in muscle glycogen (Savage et. al., A Prevalent Variant in PPP1R3A Impairs Glycogen Synthesis and Reduces Muscle Glycogen Content in Humans and Mice. PLOS Medicine. 2008; herein incorporated by reference in its entirety). PPP1R3A functions as a key activator of muscle glycogen synthase 1 (GYS1) by dephosphorylating the enzyme and maximizing activity. FIG. 1 demonstrates the pathway in which PPP1R3A (loss of function) LoF leads to reduction in muscle glycogen.

Large biobanks enable investigation of the consequences of genetic variation on many health-related phenotypes. To assess the consequences of a predicted 65% loss of muscle glycogen, association study was performed in the UK Biobank comparing phenotypes between PPP1R3A PTV carriers and non-carriers. Genetic association studies were performed using REGENIE (Mbatchou, J., Barnard, L., Backman, J. et al. Computationally efficient whole-genome regression for quantitative and binary traits. Nat Genet 53, 1097-1103, 2021), adjusted for age, sex, and the first 10 principal components of ancestry. Quantitative traits were normalized using an inverse rank normal transformation.

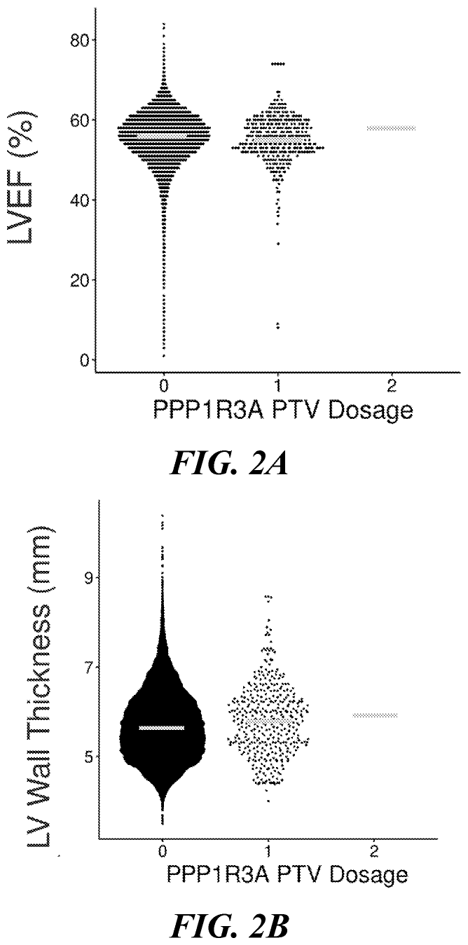

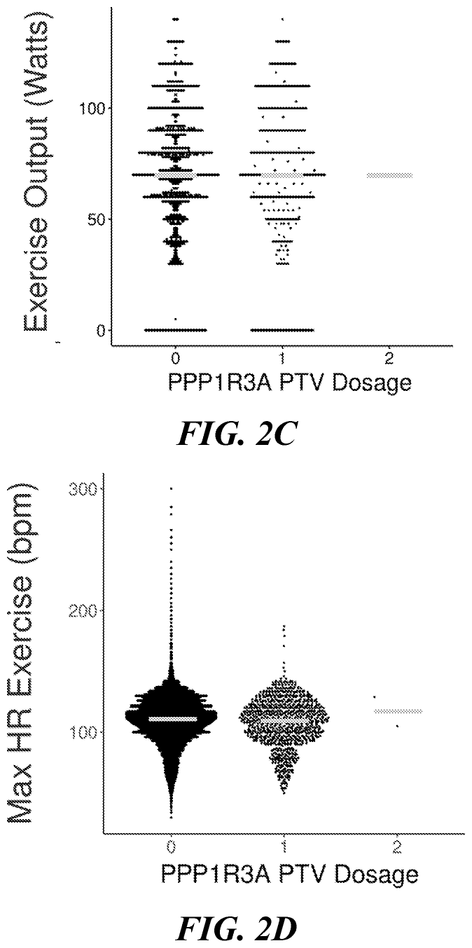

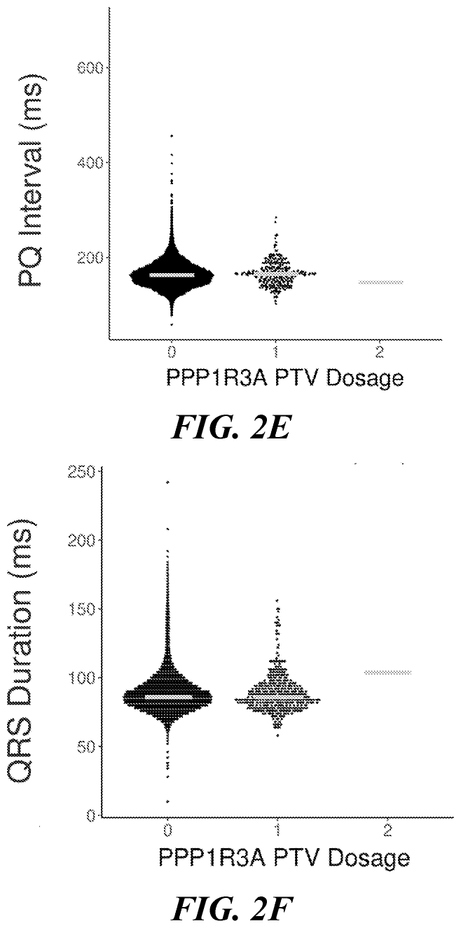

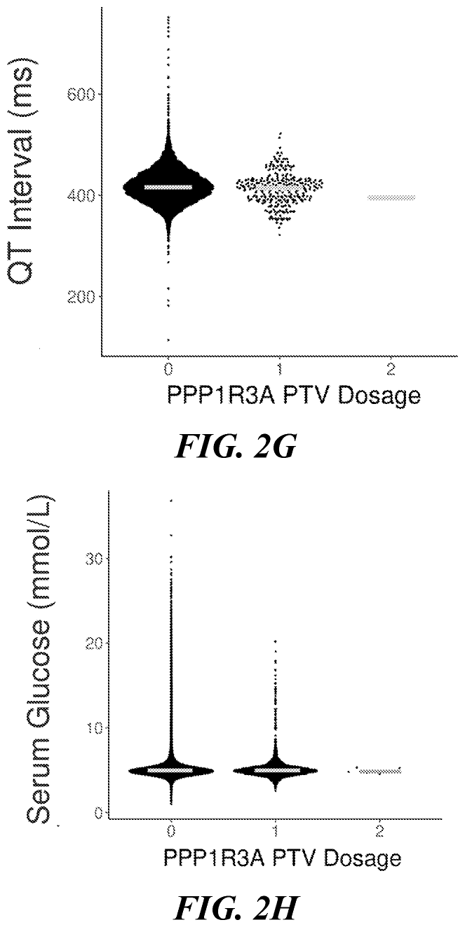

With regards to FIGS. 2A-2H, the association between PPP1R3A PTV and the quantitative phenotypes of left ventricular ejection (LVEF) (%) (FIG. 2A), left ventricle wall thickness (mm) (FIG. 2B), exercise output (watts) (FIG. 2C), max heart rate (HR) exercise (bpm) (FIG. 2D), PQ interval (ms) (FIG. 2E), QRS duration (ms) (FIG. 2F), QT interval (ms) (FIG. 2G), and serum glucose (mmol/L) (FIG. 2H), are depicted. Phenotype values are plotted by PPP1R3A dosage for UK Biobank participants. No association between PPP1R3A PTV and the quantitative phenotypes in the UK Biobank was identified.

Table 6 below lists the P-value and number of participants (N) for the results depicted in FIGS. 2A-H. No associations between PPP1R3A PTV and cardiac parameters, including left ventricular ejection fraction (p=0.871) and wall thickness (p=0.168) were identified. There was no evidence of changes in EKG cardiac conduction intervals nor in any muscle performance measurements (n=49,616), including maximum heart rate (p=0.444) and maximum workload during an exercise test (p=0.100). Further, no changes in serum glucose (p=0.71) or any other members of a panel of ˜170 serum metabolites were observed.

TABLE 6

| Phenotype | P-value | N |
|---|---|---|
| LVEF | 0.871 | 27,716 |
| LV Wall Thickness | 0.168 | 27,579 |
| Exercise Output | 0.100 | 49,616 |
| Max HR Exercise | 0.444 | 49,603 |
| QRS Duration | 0.527 | 29,507 |
| PQ Interval | 0.366 | 16,694 |
| QT Interval | 0.222 | 17,574 |
| Serum Glucose | 0.477 | 294,042 |

As shown in Table 7 below, no association between PPP1R3A PTV and key health outcomes was also observed. In addition to the phenotypes in Table 7, no phenome-wide significant associations between PPP1R3A PTV and rates of any ICD10 code with over 100 occurrences in UK Biobank was observed.

TABLE 7

| Disease | Effect (SE) | P-value | N Cases |
|---|---|---|---|
| Type 2 Diabetes | −0.094 (0.073) | 0.200 | 18,868 |
| Liver Cirrhosis | −0.041 (0.273) | 0.880 | 1,325 |
| Heart Failure | −0.061 (0.127) | 0.630 | 6,117 |

After performing an extensive Phenome-wide association study in UK Biobank, no significant associations between any key outcomes or phenotypes and loss of function of PPP1R3A were found. The results provided herein demonstrate that loss of function variants in the PPP1R3A gene are not associated with adverse health outcomes in a large biobank population. This suggests that partial reduction in muscle glycogen (˜65%) from birth is well tolerated and supports the potential safety of pharmacologic reduction of muscle glycogen.

All publications, patent applications, patents, and other references mentioned herein are expressly incorporated by reference in their entireties, to the same extent as if each were incorporated by reference individually.

It is to be understood that, while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula (I): 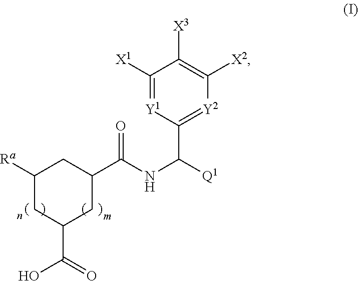
or a stereoisomer or tautomer thereof, or a pharmaceutically acceptable salt of any of the foregoing, wherein:
m is 0, or 1, and nis 0, 1, or 2, wherein m+n is an integer from 1 to 2;
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl; and
R^{a} is H, halo, —OH, or —NH—C(O)—C₁₋₆alkoxy.

2. The compound of claim 1, or a stereoisomer or tautomer thereof, or a pharmaceutically acceptable salt of any of the foregoing, wherein the compounds of formula (I) has a stereochemical configuration of

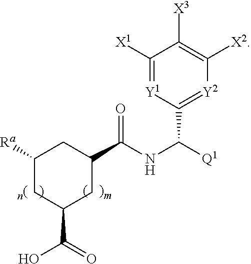

3. The compound of claim 1 or claim 2, or a stereoisomer or tautomer thereof, or a pharmaceutically acceptable salt of any of the foregoing, wherein m is 0 and nis 1.

4. The compound of claim 1 or claim 2, or a stereoisomer or tautomer thereof, or a pharmaceutically acceptable salt of any of the foregoing, wherein m is 1 and n is 0.

5. The compound of any one of claims 1-4, or a stereoisomer or tautomer thereof, or a pharmaceutically acceptable salt of any of the foregoing, wherein Y¹ and Y² are each CH.

6. The compound of any one of claims 1-5, or a stereoisomer or tautomer thereof, or a pharmaceutically acceptable salt of any of the foregoing, wherein 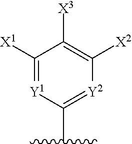
of formula (I) is selected from the group consisting of

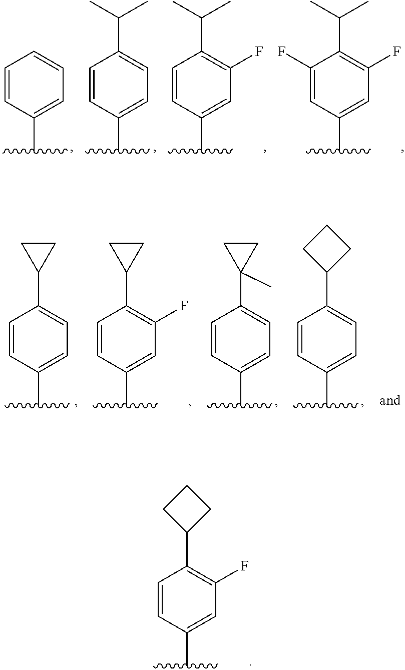

7. The compound of any one of claims 1-4, or a stereoisomer or tautomer thereof, or a pharmaceutically acceptable salt of any of the foregoing, wherein one of Y¹ and Y² is N and the other of Y¹ and Y² is CH.

8. The compound of any one of claim 1-4 or 7, or a stereoisomer or tautomer thereof, or a pharmaceutically acceptable salt of any of the foregoing, wherein 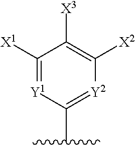
of formula (I) is selected from the group consisting of

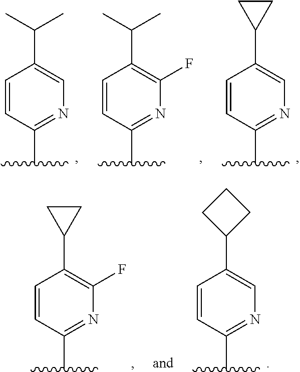

9. The compound of any one of claim 1-5, or 7, or a stereoisomer or tautomer thereof, or a pharmaceutically acceptable salt of any of the foregoing, wherein X¹ and X² are each independently H or F.

10. The compound of any one of claim 1-5, 7, or 9, or a stereoisomer or tautomer thereof, or a pharmaceutically acceptable salt of any of the foregoing, wherein X³ is H, C₁₋₃alkyl, or C₃₋₆cycloalkyl, wherein the C₃₋₆cycloalkyl of X³ is optionally substituted with one or more C₁₋₃alkyl.

11. The compound of any one of claims 1-10, or a stereoisomer or tautomer thereof, or a pharmaceutically acceptable salt of any of the foregoing, wherein Q¹ is C₆₋₁₀cycloalkyl, wherein the C₆₋₁₀cycloalkyl of Q¹ is optionally substituted with one or more C₁₋₆alkyl.

12. The compound of any one of claims 1-11, or a stereoisomer or tautomer thereof, or a pharmaceutically acceptable salt of any of the foregoing, wherein Q¹ is

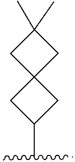 [SMILES: *C1CC2(C1)CC(C)(C)C2]

13. The compound of any one of claims 1-10, or a stereoisomer or tautomer thereof, or a pharmaceutically acceptable salt of any of the foregoing, wherein Q¹ is C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl.

14. The compound of any one of claim 1-10, or 13, or a stereoisomer or tautomer thereof, or a pharmaceutically acceptable salt of any of the foregoing, wherein Q¹ is phenyl, wherein the phenyl of Q¹ is optionally substituted with one or more R^{b}, wherein each R^{b} is independently —CH₃, —OCH₃, —NH—C(O)—NH₂, —NH—C(O)-(3-6 membered heterocyclyl), or 5-10 membered heteroaryl, wherein the 5-10 membered heteroaryl of R^{b} is optionally substituted with one or more —CH₃.

15. The compound of any one of claim 1-10, 13, or 14, or a stereoisomer or tautomer thereof, or a pharmaceutically acceptable salt of any of the foregoing, wherein Q¹ is selected from the group consisting of

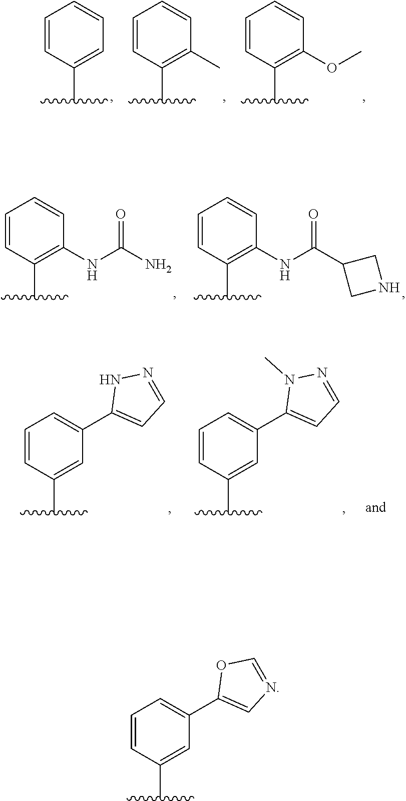

16. The compound of any one of claims 1-10, or a stereoisomer or tautomer thereof, or a pharmaceutically acceptable salt of any of the foregoing, wherein Q¹ is 3-15 membered heterocyclyl, wherein the 3-15 membered heterocyclyl of Q¹ is optionally substituted with one or more oxo or C₁₋₆alkyl.

17. The compound of any one of claim 1-10, or 16, or a stereoisomer or tautomer thereof, or a pharmaceutically acceptable salt of any of the foregoing, wherein Q¹ is selected from the group consisting of

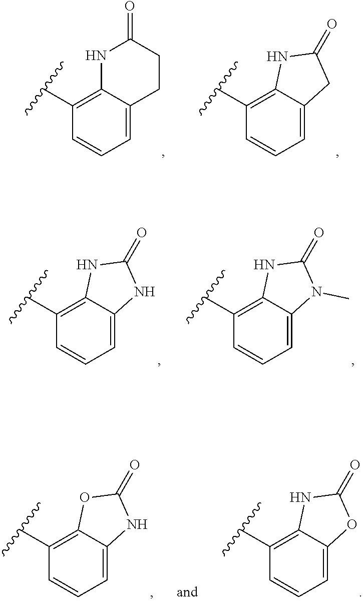 [SMILES: *c1cccc2c1NC(=O)CC2 ; *c1cccc2c1NC(=O)C2 ; *c1cccc2[nH]c(=O)[nH]c12 ; *c1cccc2c1[nH]c(=O)n2C ; *c1cccc2[nH]c(=O)oc12 ; *c1cccc2oc(=O)[nH]c12]

18. The compound of any one of claims 1-10, or a stereoisomer or tautomer thereof, or a pharmaceutically acceptable salt of any of the foregoing, wherein Q¹ is 5-10 membered heteroaryl, wherein the 5-10 membered heteroaryl of Q¹ is optionally substituted with one or more —NH₂.

19. The compound of any one of claim 1-10, or 18, or a stereoisomer or tautomer thereof, or a pharmaceutically acceptable salt of any of the foregoing, wherein Q¹ is selected from the group consisting of

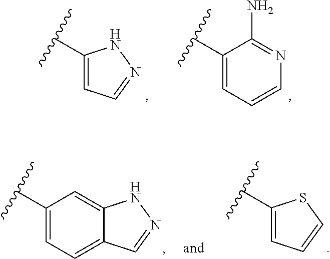 [SMILES: *c1ccn[nH]1 ; *c1cccnc1N ; *c1ccc2cn[nH]c2c1 ; *c1cccs1]

20. The compound of any one of claims 1-19, or a stereoisomer or tautomer thereof, or a pharmaceutically acceptable salt of any of the foregoing, wherein R^{a} is H, F, —OH, or —NH—C(O)—C₁₋₄alkoxy.

21. The compound of any one of claims 1-20, or a stereoisomer or tautomer thereof, or a pharmaceutically acceptable salt of any of the foregoing, wherein R^{a} is H.

22. The compound of any one of claims 1-20, or a stereoisomer or tautomer thereof, or a pharmaceutically acceptable salt of any of the foregoing, wherein R^{a} is F.

23. The compound of any one of claim 1-10, 13, 15, or 20-22, or a stereoisomer or tautomer thereof, or a pharmaceutically acceptable salt of any of the foregoing, wherein the compound is of formula (I-A): 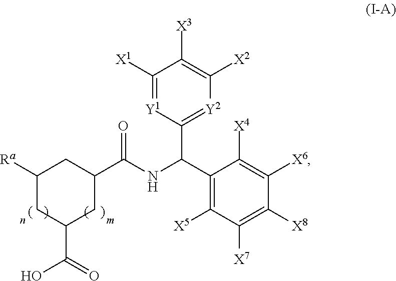
or a stereoisomer or tautomer thereof, or a pharmaceutically acceptable salt of any of the foregoing, wherein
either:
i. X⁴⁻⁸ are each independently H, C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl; or
ii. X⁶ is taken together with either of X⁴ or X⁸, and the atoms to which they are attached, to form ring A, wherein ring A is
3-3-9 membered heterocyclyl, wherein the 3-9 membered heterocyclyl of ring A is optionally substituted with one or more oxo or C₁₋₆alkyl, and wherein X³, X⁷, and the other of X⁴ or X⁸ are each independently H, oxo or C₁₋₆alkyl, or
5-14 membered heteroaryl, wherein the 5-14 membered heteroaryl of ring A is optionally substituted with one or more —NH₂, and wherein the 5-14 membered heteroaryl of ring A contains at least 1 annular N when m is 1, and wherein X⁵, X⁷, and the other of X⁴ or X⁸ are each independently H, or more —NH₂; or
iii. X⁷ is taken together with either of X⁵ or X⁸, and the atoms to which they are attached, to form ring A, wherein ring A is
3-9 membered heterocyclyl, wherein the 3-9 membered heterocyclyl of ring A is optionally substituted with one or more oxo or C₁₋₆alkyl, and wherein X⁴, X⁶, and the other of X⁵ or X⁸ are each independently H, oxo or C₁₋₆alkyl, or
5-14 membered heteroaryl, wherein the 5-14 membered heteroaryl of ring A is optionally substituted with one or more —NH₂, and wherein the 5-14 membered heteroaryl of ring A contains at least 1 annular N when m is 1, and wherein X⁴, X⁶, and the other of X⁵ or X⁸ are each independently H, or —NH₂.

24. The compound of any one of claims 1-15, or a stereoisomer or tautomer thereof, or a pharmaceutically acceptable salt of any of the foregoing, wherein one of X⁴⁻⁸ is C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl is independently optionally substituted with one or more C₁₋₆alkyl and the others of X⁴⁻⁸ are each independently H.

25. The compound of any one of claim 1-10, 13, 15, or 20-23, or a stereoisomer or tautomer thereof, or a pharmaceutically acceptable salt of any of the foregoing, wherein the compound is of formula (I-B): 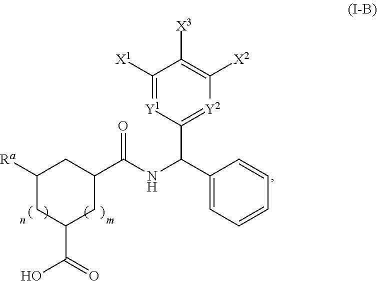
or a stereoisomer or tautomer thereof, or a pharmaceutically acceptable salt of any of the foregoing.

26. The compound of any one of claim 1-10, 13, 15, or 20-23, or a stereoisomer or tautomer thereof, or a pharmaceutically acceptable salt of any of the foregoing, wherein the compound is of formula (I-C): 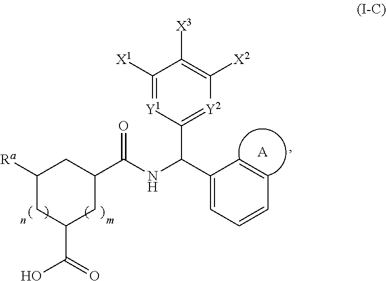
or a stereoisomer or tautomer thereof, or a pharmaceutically acceptable salt of any of the foregoing, wherein ring A is
3-9 membered heterocyclyl, wherein the 3-9 membered heterocyclyl of ring A is optionally substituted with one or more oxo or C₁₋₆alkyl, or
5-14 membered heteroaryl, wherein the 5-14 membered heteroaryl of ring A is optionally substituted with one or more —NH₂, and wherein the 5-14 membered heteroaryl of ring A contains at least 1 annular N when m is 1.

27. The compound of claim 1, or a stereoisomer or tautomer thereof, or a pharmaceutically acceptable salt of any of the foregoing, wherein the compound, or a stereoisomer or tautomer thereof, or a pharmaceutically acceptable salt of any of the foregoing, is selected from the compounds of Table 1.

28. A process for preparing a compound of any one of claims 1-27, or a stereoisomer or tautomer thereof, or a pharmaceutically acceptable salt of any of the foregoing, wherein the process comprises:
(a) reacting a compound of formula (I-1): 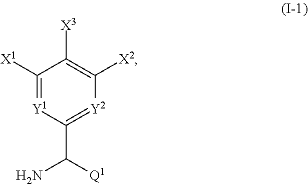
or a salt thereof, wherein
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl; and
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl;
with a compound of formula (I-2): 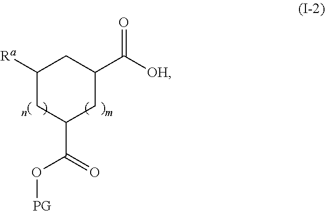
m is 0, or 1, and nis 0, 1, or 2, wherein m+n is an integer from 1 to 2;
R^{a} is H, halo, —OH, or —NH—C(O)—C₁₋₆alkoxy;
and PG is a protecting group;
in the presence of a coupling reagent, to provide a compound of formula (I-3): 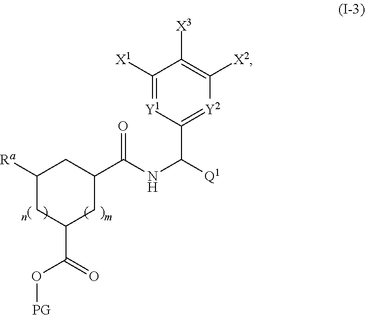
wherein
m is 0, or 1, and n is 0, 1, or 2, wherein m+n is an integer from 1 to 2;
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₄alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl;
R^{a} is H, halo, —OH, or —NH—C(O)—C₁₋₆alkoxy; and
PG is a protecting group;
followed by
(b) contacting the compound of formula (I-3) with deprotecting agent to provide a compound of any one of claims 1-27.

29. A process for preparing a compound of any one of claims 1-27, or a stereoisomer or tautomer thereof, or a pharmaceutically acceptable salt of any of the foregoing, wherein the process comprises:
reacting a compound of formula (I-1): 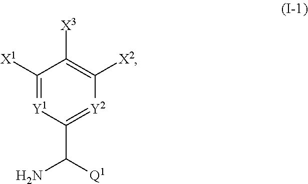
or a salt thereof, wherein
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl; and
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl;
with a compound of formula (I-4): 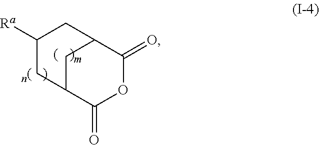
wherein,
m is 0, or 1, and nis 0, 1, or 2, wherein m+n is an integer from 1 to 2;
Y¹ and Y² are each CH, or
one of Y¹ and Y² is N and the other of Y¹ and Y² is CH;
X¹ and X² are each independently H or halo;
X³ is H, C₁₋₆alkyl, or C₃₋₁₀cycloalkyl, wherein the C₃₋₁₀cycloalkyl of X³ is optionally substituted with one or more C₁₋₆alkyl;
Q¹ is:
(i) C₆₋₁₀cycloalkyl, wherein the C₆₋₁₀cycloalkyl of Q¹ is optionally substituted with one or more C₁₋₆alkyl,
(ii) C₆₋₂₀aryl, wherein the C₆₋₂₀aryl of Q¹ is optionally substituted with one or more R^{b}, wherein each R^{b} is independently C₁₋₆alkyl, —C₁₋₆alkoxy, —NH—C(O)—NH₂, —NH—C(O)-(3-15 membered heterocyclyl), or 5-20 membered heteroaryl, wherein the 5-20 membered heteroaryl of R^{b} is optionally substituted with one or more C₁₋₆alkyl,
(iii) 3-15 membered heterocyclyl, wherein the 3-15 membered heterocyclyl of Q¹ is optionally substituted with one or more oxo or C₁₋₆alkyl, or
(iv) 5-20 membered heteroaryl, wherein the 5-20 membered heteroaryl of Q¹ is optionally substituted with one or more —NH₂, and wherein the 5-20 membered heteroaryl of Q¹ contains at least 1 annular N when m is 1,
provided that, when X³ is H, then Q¹ is C₆₋₁₀cycloalkyl, wherein the C₆₋₁₀cycloalkyl of Q¹ is optionally substituted with one or more C₁₋₆alkyl; and
R^{a} is H, halo, —OH, or —NH—C(O)—C₁₋₆alkoxy;
in the presence of a coupling reagent to provide a compound of any one of claims 1-27.

30. A pharmaceutical composition comprising (i) a compound of any one of claims 1-27, or a stereoisomer or tautomer thereof, or a pharmaceutically acceptable salt of any of the foregoing, and (ii) one or more pharmaceutically acceptable excipients.

31. A method of treating a GYS1-mediated disease, disorder, or condition in an individual in need thereof, comprising administering to the individual a compound of any one of claims 1-27, or a stereoisomer or tautomer thereof, or a pharmaceutically acceptable salt of any of the foregoing, or a pharmaceutical composition of claim 30.

32. The method of claim 31, wherein the disease, disorder, or condition is a glycogen storage disorder (GSD).

33. The method of claim 31 or claim 32, wherein the disease, disorder, or condition is selected from the group consisting of Pompe disease, Cori disease (GSD III), adult polyglucosan body disease (APBD), and Lafora disease.

34. The method of any one of claims 31-33, wherein the disease, disorder, or condition is Pompe disease.

35. The method of claim 31, wherein the disease, disorder, or condition is cancer.

36. The method of claim 31 or claim 35, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

37. The method of claim 31, wherein the individual has a GAA mutation.

38. The method of claim 37, wherein the GAA mutation is a loss-of-function mutation.

39. A kit, comprising (i) a compound of any one of claims 1-27, or a stereoisomer or tautomer thereof, or a pharmaceutically acceptable salt of any of the foregoing, or a pharmaceutical composition of claim 30, and (ii) instructions for use in treating an GYS1-mediated disease, disorder, or condition in an individual in need thereof.

40. The kit of claim 39, wherein the disease, disorder, or condition is a glycogen storage disorder (GSD).

41. The kit of claim 39 or claim 40, wherein the disease, disorder, or condition is selected from the group consisting of Pompe disease, Cori disease (GSD III), adult polyglucosan body disease (APBD), and Lafora disease.

42. The kit of any one of claims 39-41, wherein the disease, disorder, or condition is Pompe disease.

43. The kit of claim 42, wherein the disease, disorder, or condition is cancer.

44. The kit of claim 39 or claim 43, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

45. The kit of claim 39, wherein the individual has a GAA mutation.

46. The kit of claim 45, wherein the GAA mutation is a loss-of-function mutation.

47. A method of modulating GYS1 in a cell, comprising exposing the cell to a composition comprising an effective amount of a compound of any one of claims 1-27, or a stereoisomer or tautomer thereof, or a pharmaceutically acceptable salt of any of the foregoing, or the pharmaceutical composition of claim 30.

48. A method of inhibiting GYS1 in a cell, comprising exposing the cell to a composition comprising an effective amount of a compound of any one of claims 1-27, or a stereoisomer or tautomer thereof, or a pharmaceutically acceptable salt of any of the foregoing, or the pharmaceutical composition of claim 30.

49. A method of reducing tissue glycogen stores in an individual in need thereof, comprising administering to the individual an effective amount of a compound of any one of claims 1-27, or a stereoisomer or tautomer thereof, or a pharmaceutically acceptable salt of any of the foregoing, or a pharmaceutical composition of claim 30.

50. A method of treating a GYS1-mediated disease, disorder, or condition in an individual in need thereof, comprising subjecting the individual to glycogen substrate reduction therapy, wherein the glycogen substrate reduction therapy comprises administering to the individual an effective amount of a compound of any one of claims 1-27, or a stereoisomer or tautomer thereof, or a pharmaceutically acceptable salt of any of the foregoing, or the pharmaceutical composition of claim 30.

51. The method of claim 50, comprising subjecting the individual to glycogen substrate reduction therapy in combination with enzyme replacement therapy.

52. The method of claim 51, wherein the enzyme replacement therapy is selected from the group consisting of alglucosidase alfa (human recombinant alpha-glucosidase (human GAA)) Myozyme and Lumizyme.

53. The method of any one of claims 50-52, wherein the disease, disorder, or condition is a glycogen storage disorder (GSD).

54. The method of any one of claims 50-53, wherein the disease, disorder, or condition is selected from the group consisting of Pompe disease, Cori disease (GSD III), adult polyglucosan body disease (APBD), and Lafora disease.

55. The method of any one of claims 50-54, wherein the disease, disorder, or condition is Pompe disease.

56. The method of any one of claims 50-52, wherein the disease, disorder, or condition is cancer.

57. The method of any one of claim 50-52, or 56, wherein the disease, disorder, or condition is selected from the group consisting of Ewing sarcoma (ES), clear cell renal cell carcinoma (ccRCC), glycogen rich clear cell carcinoma (GRCC) breast cancer, non-small-cell lung carcinoma (NSCLC), and acute myeloid leukemia (AML).

58. The method of any one of claims 50-52, wherein the individual has a GAA mutation.

59. The method of claim 58, wherein the GAA mutation comprises a loss-of-function mutation.

60. The method of any one of claims 47-49 wherein the compound is selective for GYS1 over GYS2.

61. The method of claim 60, wherein the compound is greater than 500 or 1,000 or 1,500 or 1,700-fold selective for GYS1 over GYS2.

62. The method of any one of claim 31-38 or 47-61, comprising reducing the level of glycogen in skeletal muscle.

63. A compound of any one of claims 1-27, or a stereoisomer or tautomer thereof, or a pharmaceutically acceptable salt of any of the foregoing, or a pharmaceutical composition of claim 30, for use in treating a GYS1-mediated disease, disorder, or condition in an individual in need thereof.

64. A compound of any one of claims 1-27, or a stereoisomer or tautomer thereof, or a pharmaceutically acceptable salt of any of the foregoing, or the pharmaceutical composition of claim 30, for use in modulating GYS1 in a cell.

65. A compound of any one of claims 1-27, or a stereoisomer or tautomer thereof, or a pharmaceutically acceptable salt of any of the foregoing, or the pharmaceutical composition of claim 30, for use in inhibiting GYS1 in a cell.

66. A compound of any one of claims 1-27, or a stereoisomer or tautomer thereof, or a pharmaceutically acceptable salt of any of the foregoing, or a pharmaceutical composition of claim 30, for use in reducing tissue glycogen stores in an individual in need thereof.

67. A compound of any one of claims 1-27, or a stereoisomer or tautomer thereof, or a pharmaceutically acceptable salt of any of the foregoing, or the pharmaceutical composition of claim 30, for use in a glycogen substrate reduction therapy for treating a GYS1-mediated disease, disorder, or condition in an individual in need thereof.

68. Use of a compound of any one of claims 1-27, or a stereoisomer or tautomer thereof, or a pharmaceutically acceptable salt of any of the foregoing, or a pharmaceutical composition of claim 30, in the manufacture of a medicament for use in treating a GYS1-mediated disease, disorder, or condition in an individual in need thereof.

69. Use of a compound of any one of claims 1-27, or a stereoisomer or tautomer thereof, or a pharmaceutically acceptable salt of any of the foregoing, or the pharmaceutical composition of claim 30, in the manufacture of a medicament for use in modulating GYS1 in a cell.

70. Use of a compound of any one of claims 1-27, or a stereoisomer or tautomer thereof, or a pharmaceutically acceptable salt of any of the foregoing, or the pharmaceutical composition of claim 30, in the manufacture of a medicament for use in inhibiting GYS1 in a cell.

71. Use of a compound of any one of claims 1-27, or a stereoisomer or tautomer thereof, or a pharmaceutically acceptable salt of any of the foregoing, or a pharmaceutical composition of claim 30, in the manufacture of a medicament for use in reducing tissue glycogen stores in an individual in need thereof.

72. Use of a compound of any one of claims 1-27, or a stereoisomer or tautomer thereof, or a pharmaceutically acceptable salt of any of the foregoing, or the pharmaceutical composition of claim 30, in the manufacture of a medicament for use in a glycogen substrate reduction therapy for treating a GYS1-mediated disease, disorder, or condition in an individual in need thereof.
